(19) United States
(12) Patent Application Publication
(10) Pub. No.: US 2021/0000953 A1
(43) Pub. Date: Jan. 7, 2021

(54) USE OF IMMUNE CHECKPOINT INHIBITORS IN CENTRAL NERVOUS SYSTEMS NEOPLASMS

(71) Applicant: Bristol-Myers Squibb Company, Princeton, NJ (US)

(72) Inventors: Vladimir CORIC, Madison, CT (US); Shinta CHENG, Pennington, NJ (US)

(73) Assignee: Bristol-Myers Squibb Company, Princeton, NJ (US)

(21) Appl. No.: 16/933,547

(22) Filed: Jul. 20, 2020

Related U.S. Application Data
(63) Continuation of application No. 15/536,785, filed on Jun. 16, 2017, now abandoned.
(60) Provisional application No. 62/092,783, filed on Dec. 16, 2014.

(51) Int. Cl. A61K 39/395; C07K 16/28; A61P 25/00; A61P 35/00; A61K 9/00

(52) U.S. Cl. CPC A61K 39/39558; C07K 16/2818; A61K 2039/505; A61P 35/00; A61K 9/0019; A61P 25/00

(57) ABSTRACT

This disclosure provides a method for treating glioma in a subject comprising administering to the subject an anti-Programmed Death-1 (PD-1) antibody. In some embodiments, this invention relates to methods for treating glioma in a subject comprising administering to the subject a combination of an anti-cancer agent which is an anti-Programmed Death-1 (PD-1) antibody and another anti-cancer agent such as an anti-Cytotoxic T-Lymphocyte Antigen-4 (CTLA-4) antibody.

### CROSS REFERENCE TO RELATED APPLICATIONS

The present application is a continuation of U.S. application Ser. No. 15/536,785, which is the US National Phase entry of International Application No. PCT/US2015/066177 filed on Dec. 16, 2015, which claims benefit to U.S. Provisional Application Nos. 62/092,783, filed Dec. 16, 2014 and 62/261,130, filed Nov. 30, 2015; each of which is incorporated herein by reference herein in its entirety.

### BACKGROUND OF THE INVENTION

Throughout this application, various publications are referenced in parentheses by author name and date, or by patent No. or patent Publication No. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

This invention relates to methods for treating a glioma in a subject comprising administering to the subject an anti-Programmed Death-1 (PD-1) antibody. In some embodiments, this invention relates to methods for treating a glioma in a subject comprising administering to the subject a combination of an anti-Programmed Death-1 (PD-1) antibody and another anti-cancer agent such as an anti-Cytotoxic T-Lymphocyte Antigen-4 (CTLA-4) antibody. In some embodiments, the glioma is a glioblastoma (GBM).

### BACKGROUND OF THE INVENTION

Gliomas are neoplasms or tumors that arise in the brain or spinal cord, most commonly in the brain. Gliomas are named for the cell types with which they share histological features. GBM is the most frequent primary brain tumor in adults and accounts for approximately 60-70% of all malignant gliomas. (Wen P. Y. et al., N Engl J Med. 359(5):492-507 (2008)). GBMs are associated with a particularly aggressive clinical course with high morbidity and mortality. In the United States alone, about twelve to fourteen thousand new cases of GBM multiform are diagnosed each year.

Median survival of GBM without treatment is 4½ months. Despite standard first-line treatment with neurosurgery, radiation therapy (RT) and/or temozolomide, median survival is approximately 12-15 months, and nearly all cases recur. (Chang S. M. at al. Neurosurg Focus. 20(4):E4 Review (2006); Johnson, D. R. and O'Neill, B. P., J. Neurooncol. 107(2):359-64 (2012)) Avastin (bevacizumab) is an anti-angiogenic therapy that has been approved to treat recurrent GBM. However, preliminary studies showed that, while treatment with Avastin increases progression-free survival, it does not significantly increase overall survival for newly diagnosed GBM above and beyond standard of care therapies when Avastin is used in combination with standard of care therapies (radiotherapy plus temozolomide). (Chinot et al., N Engl J Med 370:709-722 (2014); Gilbert et al., N Engl J Med 370:699-708 (2014)) Furthermore, studies have indicated that patients treated with bevacizumab experience reduced cognitive function and quality of life. (See http://www.mdanderson.org/newsroom/news-releases/2013/patients-treated-with-bevacizumab.html, last visited Dec. 15, 2015) Thus, there is no known curative treatment for GBM. Furthermore, GBM treatment is particularly difficult to treat because some drugs cannot traverse the blood brain barrier. Therefore, there is an urgent need for novel treatment interventions to improve clinical outcomes and quality of life for patients suffering from GBM.

### SUMMARY OF THE INVENTION

The present invention relates to a method for treating a subject afflicted with a glioma comprising administering to the subject a therapeutically effective amount of an antibody or an antigen-binding portion thereof that binds specifically to a Programmed Death-1 (PD-1) receptor and inhibits PD-1 activity (“anti-PD-1 antibody”). In one embodiment, the glioma is selected from the group consisting of: astrocytoma, brainstem glioma, ependymoma, mixed glioma, oligodendroglioma, optic nerve glioma, or any combination thereof. In another embodiment, the glioma is GBM.

In other embodiments, the anti-PD-1 antibody cross-competes with nivolumab for binding to human PD-1. In some embodiments, the anti-PD-1 antibody is a chimeric, humanized or human monoclonal anti-PD-1 antibody. In one embodiment, the anti-PD-1 antibody is nivolumab. In another embodiment, the anti-PD-1 antibody is pembrolizumab.

In some embodiments, the subject is further treated with an anti-cancer therapy concurrently with, prior to, or after the administering of an anti-PD-1 antibody. In some embodiments, the anti-cancer therapy comprises administering an anti-cancer agent. In one embodiment, the anti-cancer therapy comprises a radiation therapy. In another embodiment, the anti-cancer therapy comprises a surgery. In a further embodiment, the surgery comprises MRI-guided laser ablation.

In some embodiments, the anti-cancer agent comprises a second antibody or an antigen-binding portion thereof. In one embodiment, the second antibody or an antigen-binding portion thereof binds specifically to Cytotoxic T-Lymphocyte Antigen-4 (CTLA-4) and inhibits CTLA-4 activity (“anti-CTLA-4 antibody”). In another embodiment, the anti-CTLA-4 antibody cross-competes with ipilimumab for binding to human CTLA-4. In other embodiments, the anti-CTLA-4 antibody is a chimeric, humanized or human monoclonal antibody. In one embodiment, the anti-CTLA-4 antibody is ipilimumab.

In some embodiments, the anti-PD-1 antibody is administered as monotherapy at a dose of about 3 mg/kg body weight. In other embodiments, the anti-PD-1 antibody is administered at a dose of about 3 mg/kg body weight in combination with an anti-CTLA-4 antibody, which is administered at a dose of about 1 mg/kg body weight.

In certain embodiments, the present invention is directed to any method disclosed herein comprising: 1) an induction phase, wherein the anti-PD-1 and anti-CTLA-4 antibodies are administered in combination in 2, 4, 6, 8 or 10 doses, each dose ranging from at least about 0.1 mg/kg to at least about 10.0 mg/kg body weight administered at least once about every 2, 3, or 4 weeks; 2) followed by a maintenance phase, wherein no anti-CTLA-4 antibody is administered and the anti-PD-1 antibody is repeatedly administered at a dose ranging from at least about 0.1 mg/kg to at least about 10 mg/kg at least once about every 2, 3 or 4 weeks.

### BRIEF DESCRIPTION OF THE DRAWINGS

FIG. 1 provides a schematic representation of the study design of Cohort 1.

FIG. 2 provides a schematic representation of the study design of Cohort 1b.

FIG. 3 provides a schematic representation of the study design of Cohort 2.

FIG. 4 shows the status of Cohort 1 Arm N+I [nivolumab (1 mg/Kg)+Ipilimumab (3 mg/kg)] subjects (light bars) and Cohort 1 Arm N [Nivolumab (3 mg/kg) monotherapy] subjects (dark bars). Subject status is labeled as shown, including stable disease (SD), pseudoprogression (PseudoPrg), progressive disease (PD), partial response (PR), and death (D). Any changes from Arm N+I to Arm N (Switched to N) or discontinuation of treatment due to adverse events (D/C Tx AE) are also indicated.

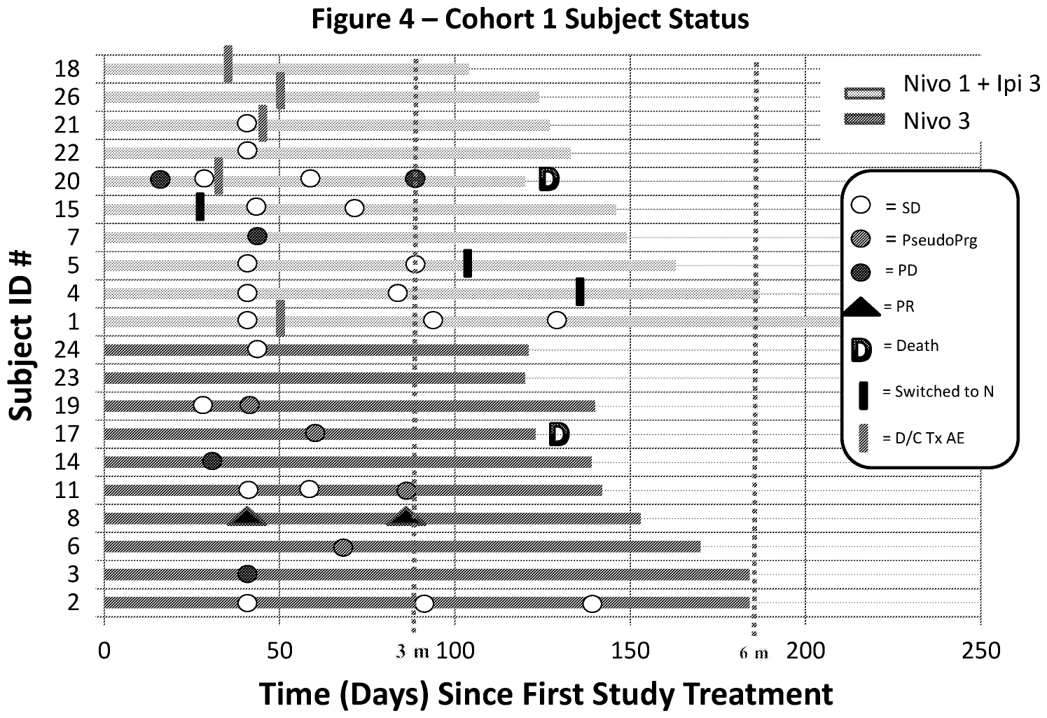

FIGS. 5A-5D show brain MRI images of a subject displaying pseudoprogression of a GBM following nivolumab monotherapy. Images on the left (FIGS. 5A and 5B) were taken at two different sections prior to study treatment. Images on the right (FIGS. 5C and 5D) were taken at similar sections following 5 doses of nivolumab monotherapy. The subject did not receive concurrent corticosteroid treatment.

FIGS. 6A-6C show resected tumor tissue histology showing periventricular necrotic tissue (FIG. 6A) and large aggregates of immune cells (FIG. 6B) in a subject showing treatment-related changes in a GBM patient following nivolumab monotherapy. FIG. 6C shows brain MRI image of the subject.

FIGS. 7A-7C show resected tumor tissue histology showing treatment-related changes including intratumoral macrophages (FIG. 7A) and CD45-positive cell aggregates (FIG. 7B) in a subject experiencing GBM pseudoprogression on MRI following nivolumab monotherapy. FIG. 7C shows brain MRI image of the subject.

FIGS. 8A-8C show resected tumor tissue histology showing CD45-positive (FIG. 8A) and CD3-positive cells (FIG. 8B) in a subject experiencing pseudoprogression of a GBM following nivolumab monotherapy. The brain MRI image is shown in FIG. 8C.

FIGS. 9A-9C show resected tumor tissue histology showing perivascular cuffing (FIG. 9A) and infiltrating cells as well as perivascular cuffing of CD45-positive cells (FIG. 9B) in a subject experiencing pseudoprogression of a GBM following nivolumab monotherapy. The brain MRI image is shown in FIG. 9C.

FIGS. 10A-10C show resected tumor tissue histology showing CD45-positive cells lining a tumor blood vessel (FIG. 10A) as well as perivascular cuffing and infiltrating of CD45 positive cells into the tumor (FIG. 10B) in a subject experiencing pseudoprogression of a GBM following nivolumab monotherapy. The brain MRI image is shown in FIG. 10C.

FIGS. 11A-11D show brain MRI images of a subject experiencing a partial response following nivolumab monotherapy. FIG. 11A shows the baseline, and FIG. 11B shows the subject after three doses of nivolumab monotherapy. FIGS. 11C and 11D show follow-up brain MRI images on two different time points while the subject remained on nivolumab.

FIG. 12 shows the study design of a clinical trial comparing nivolumab and temozolomide in combination with radiation therapy.

FIG. 13 shows the treatment schedule, including sample collection.

FIG. 14 shows peripheral blood and tumor-infiltrating T-cell analysis upon suspected disease progression in patients receiving nivolumab monotherapy.

FIGS. 15A and 15B show the maturation profile of CD4⁺ (A) and CD8⁺ (B) T lymphocytes in patients treated with nivolumab±ipilimumab.

FIG. 16 shows a time course of CD8⁺CD28⁻ T-cell proliferation and activation in a patient treatment with nivolumab+ipilimumab.

FIGS. 17A and 17B show the anti-TAA activity of TILS (A) and PBMCs (B) in a patient treated with nivolumab monotherapy.

### DETAILED DESCRIPTION OF THE INVENTION

The present invention relates to methods for treating a glioma or a glioma patient comprising administering to the patient an anti-PD-1 antibody. In some embodiments, the present invention relates to methods for treating a glioma with an anti-PD-1 antibody. In some embodiments, the anti-PD-1 antibody is administered with another anti-cancer agent.

### Terms

In order that the present disclosure may be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a therapeutic agent to a subject, using any of the various methods and delivery systems known to those skilled in the art. Exemplary routes of administration for the anti-PD-1 antibody include intravenous, intramuscular, subcutaneous, intraperitoneal, spinal or other parenteral routes of administration, for example by injection or infusion. In some embodiments, the administering route is directly to the brain or central nervous system. In embodiments, the administration to the brain or central nervous system is intracerebroventricular (ICV), intraventricular, intrathecal (IT), interstitial, epidural, intracerebral, or intranasal (olfactory pathway), administration. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the therapeutic agent is administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. A medical treatment may have one or more associated AEs and each AE may have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized Abs; human or nonhuman Abs; wholly synthetic Abs; and single chain Abs. A nonhuman antibody may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody may be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated Ab. MAbs may be produced by hybridoma, recombinant, transgenic or other techniques known to those skilled in the art.

A “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-PD-1 Ab binds specifically to PD-1 and an anti-CTLA-4 Ab binds specifically to CTLA-4.

An “antigen-binding portion” of an Ab (also called an “antigen-binding fragment”) refers to one or more fragments of an Ab that retain the ability to bind specifically to the antigen bound by the whole Ab.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

The term “glioma” refers to a tumor that arises in the brain or spinal cord, most commonly in the brain. In some embodiments, the glioma is astrocytoma, brainstem glioma, ependymoma, mixed glioma, oligodendroglioma, or optic nerve glioma. In certain embodiments, glioma includes, but are not limited to, acoustic neuroma, e.g., astrocytoma: Grade I—Pilocytic Astrocytoma, Grade II—Low-grade Astrocytoma, Grade III—Anaplastic Astrocytoma, Grade IV—Glioblastoma (GBM). Other types of glioma includes, but are not limited to, Chordoma, CNS lymphoma, Craniopharyngioma, Brain Stem Glioma, Ependymoma, Mixed Glioma, Optic Nerve Glioma, Subependymoma, Medulloblastoma, Meningioma, Metastatic Brain Tumors, Oligodendroglioma, Pituitary Tumors, Primitive Neuroectodermal (PNET), Schwannoma, Juvenile Pilocytic Astrocytoma (JPA), Pineal Tumor, and/or Rhabdoid Tumor.

In certain embodiments, the GBM is a tumor O-6-methylguanine DNA methyltransferase (MGMT) GBM. In some embodiments, the MGMT GBM is methylated. In other embodiments, the MGMT GBM is unmethylated. In some embodiments, the anti-PD-1 antibody for the present methods is used to treat a subject who has failed a standard of therapy, e.g., an alkylating agent, e.g., temozolomide, or who is identified as having a type of GBM that is not responsive to a standard of therapy, e.g., an alkylating agent, e.g., temozolomide. Subjects having unmethylated MGMT (tumor O-6-methylguanine DNA methyltransferase) GBM represent the GBM population with the highest unmet medical need, as the subjects are less responsive to alkylating agents. This is because the unmethylated MGMT expressed in the subjects can repair the DNA damage to the tumors caused by alkylating agents, e.g., temozolomide, while the methylated MGMT expressed in the subjects having methylated MGMT GBM cannot repair the DNA damage to the tumor and thus can make the tumor more susceptible to the alkylating agents.

The term “glioblastoma,” “glioblastoma multiforme,” or “GBM” refers to a type of cancer that is a subset of astrocytomas, e.g., Grade IV Astrocytoma. GBMs include the GBM variants: giant cell GBM and gliosarcoma as well as the four subtypes of GBM: classical, neural, proneural, and mesenchymal. GBM tumors can be primary (de novo) or secondary.

“Cytotoxic T-Lymphocyte Antigen-4 (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

The term “immunotherapy” refers to the treatment of a subject afflicted with, or at risk of contracting or suffering a recurrence of, a disease by a method comprising inducing, enhancing, suppressing or otherwise modifying an immune response.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

“Programmed Death Ligand-1 (PD-L1)” is one of two cell surface glycoprotein ligands for PD-1 (the other being PD-L2) that down regulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 20%, by at least about 30%, by at least about 40%, by at least about 50%, by at least about 60%, by at least about 70%, or by at least about 80% relative to untreated subjects.

In other embodiments of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the term “flat dose” as used herein means a dose that is administered to a patient without regard for the weight or body surface area (BSA) of the patient. The flat dose is therefore not provided as a mg/kg dose, but rather as an absolute amount of the agent (e.g., an anti-PD-1 antibody). For example, a 60 kg person and a 100 kg person would receive the same dose of the therapeutic agent (e.g., 240 mg of an anti-PD-1 antibody).

The term “weight based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The use of the term “fixed dose” with regard to a method of the invention means that two or more different antibodies in a single composition are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody to mg second antibody. For example, the 3:1 ratio of a first antibody and a second antibody can mean that a vial can contain about 240 mg of the first antibody and 80 mg of the second antibody or about 3 mg/ml of the first antibody and 1 mg/ml of the second antibody.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Methods of the Invention

The present invention is directed to a method for treating a glioma or a subject afflicted with glioma comprising administering to the subject a therapeutically effective amount of a Programmed Death-1 (PD-1) antagonist, e.g., an antibody or an antigen-binding portion thereof that binds specifically to PD-1 and inhibits PD-1 activity (“anti-PD-1 antibody”) or a Programmed Death-Ligand 1 (PD-L1) antagonist, e.g., an antibody or an antigen-binding portion thereof that binds specifically to PD-L1 and inhibits PD-L1 activity (“anti-PD-L1 antibody). In one embodiment, the glioma is selected from astrocytoma, brainstem glioma, ependymoma, mixed glioma, oligodendroglioma, and optic nerve glioma. In another embodiment, the glioma is a GBM. In other embodiments, the glioma is gliosarcoma. In some embodiments, the glioma is newly diagnosed GBM or gliosarcoma. In certain embodiments, the glioma is recurrent malignant GBM. In certain embodiments, the GBM is a MGMT (tumor O-6-methylguanine DNA methyltransferase) GBM. In some embodiments, the MGMT GBM is methylated. In other embodiments, the MGMT GBM is unmethylated. In some embodiments, the glioma tumor expresses PD-L1. In other embodiments, the glioma tumor expresses PD-L2. In other embodiments, the glioma tumor expresses PD-L1 and PD-L2. In some embodiments, the glioma tumor does not express PD-L1, PD-L2, or both.

In some embodiments, the invention includes a method of treating a glioma or a subject afflicted with glioma comprising a mono-therapy (administering an anti-PD-1 antibody or an anti-PD-L1 Ab).

In other embodiments, the invention includes a method of treating a glioma or a subject afflicted with glioma comprising administering an anti-PD-1 antagonist or an anti-PD-L1 antagonist or an anti-PD-1 antagonist or an anti-PD-L1 antagonist in combination with one or more anti-cancer agents described herein to treat cancer. In some embodiments, the glioma is a GBM. An “anti-PD-1 antagonist” or an “anti-PD-L1 antagonist” as referred herein includes any molecule that inhibits interaction between PD-1 (receptor) and PD-L1 (ligand) such that the signal pathway of PD-1/PD-L1 is blocked. In one embodiment, an anti-PD-1 antagonist is an anti-PD-L1 antibody. In another embodiment, an anti-PD-1 antagonist or the anti-PD-L1 antagonist is a soluble PD-1 protein or PD-L1 protein. In other embodiments, an anti-PD-1 antagonist or an anti-PD-L1 antagonist is a PD-1-Fc fusion protein or a PD-L1-Fc fusion protein. In certain embodiments, an anti-PD-1 antagonist or an anti-PD-L1 antagonist includes an anti-PD-1 fusion protein, an anti-PD-L1 fusion protein, an antisense molecule, a small molecule, a ribozyme, or a nanobody that inhibits or prevents interaction between PD-1 and PD-L1.

In certain embodiments, the invention includes a method of treating a glioma or a subject afflicted with glioma comprising a combination therapy. In one embodiment, the present invention is directed to a method of treating a glioma or a subject afflicted with a glioma, which method comprises administering to the subject a combination of therapeutically effective amounts of: (a) an anti-PD-1 antibody or an anti-PD-L1 antibody; and (b) another anti-cancer therapy. In another embodiment, the present invention is directed to a method of treating a glioma or a subject afflicted with a glioma, which method comprises administering to the subject a combination of therapeutically effective amounts of: (a) an anti-PD-1 antibody or an anti-PD-L1 Ab; and (b) a standard of care therapy disclosed elsewhere herein.

In some embodiments, the other anti-cancer therapy is an anti-cancer agent (e.g., chemotherapy or any other therapy disclosed herein or known in the art), radiation, surgery, or a second antibody or antigen-binding portion thereof. In some embodiments, the anti-cancer agent comprises an alkylating agent, a second antibody or an antigen-binding portion thereof, or both. In some embodiments, the alkylating agent is temozolomide. In some embodiments, the anti-cancer agent is a platinum-based doublet chemotherapy, an EGFR-targeted tyrosine kinase inhibitor, or bevacizumab. In some embodiments, the second antibody or an antigen-binding portion thereof is an Ab or an antigen-binding portion thereof that binds specifically to CTLA-4 and inhibits CTLA-4 activity. In some embodiments, the second antibody or antigen-binding portion thereof is an anti-CTLA-4 antibody. In other embodiments, the subject is a human patient. In certain embodiments, the subject is a chemotherapy-naïve patient (e.g., a patient who has not previously received any chemotherapy). In other embodiments, the subject has received another cancer therapy (e.g., a chemotherapy), but is resistant or refractory to such another cancer therapy. In a particular embodiment, the invention includes a method of treating a recurrent malignant glioma in a subject in need thereof comprising administering an anti-PD-1 antibody in combination with MRI-guided Laser Ablation. In certain embodiments, the subject first undergoes MRI-guided laser ablation, which can cause disruption of peritumoral blood brain barrier and thus allow the anti-PD-1 antibody or the anti-PD-L1 antibody to pass through the blood-brain barrier.

In certain embodiments, the anti-PD-1 antibody or the anti-PD-L1 antibody is combined with focal radiation therapy at least about 1, about 2 or about 3 GY, about 2, about 3, about 4, about 5, or about 6 times per week. In some embodiments, the anti-PD-1 antibody or the anti-PD-L1 antibody is combined with focal radiation therapy at about 2 GY about 5 times per week. In other embodiments, the radiation therapy is continued for at least about 2, about 3, about 4, about 5, about 6, about 7, about 8, about 9 or about 10 weeks. In some embodiments, the radiation therapy is continued for about 6 weeks.

A combination therapy of the invention includes methods of treating newly diagnosed glioma, e.g., gliosarcoma or GBM, comprising administering an anti-PD-1 antibody or an antigen-binding portion thereof or an anti-PD-L1 antibody or an antigen-binding portion thereof, optionally in combination with an anti-CTLA-4 antibody or an antigen-binding portion thereof and/or an anti-cancer agent, e.g., an alkylating agent, e.g., temozolomide. In one embodiment, the method further comprises administering a chemoradiation to the subject prior to the administering the anti-PD-1 antibody or the anti-PD-L1 antibody, the anti-CTLA-4 antibody, or temozolomide. In another embodiment, the anti-PD-1 antibody or the anti-PD-L1 antibody, the anti-CTLA-4 antibody, and/or temozolomide are administered within one week, two weeks, three weeks, four weeks, five weeks, six weeks, seven weeks, eight weeks, or nine weeks after completion of the chemoradiation. In certain embodiments, the anti-PD-1 antibody or the anti-PD-L1 antibody is combined with focal radiation therapy and temozolomide. In some embodiments, following the anti-PD-1 antibody or the anti-PD-L1 antibody combination with focal radiation therapy and temozolomide, the anti-PD-1 antibody or the anti-PD-L1 antibody treatment is continued as maintenance. In some embodiments, following the anti-PD-1 antibody or the anti-PD-L1 antibody combination with focal radiation therapy and temozolomide, the temozolomide is continued as maintenance.

In some embodiments, the PD-1 antibody or an antigen-binding portion thereof or the anti-PD-L1 antibody or an antigen-binding portion thereof and/or the anti-CTLA-4 antibody or an antigen-binding portion thereof are administered in combination with one or more additional anti-cancer agents selected from the group consisting of a LAG3 (Lymphocyte activation gene 3 protein) antagonist (e.g., an anti-LAG3 antibody), a CD137 antagonist (e.g., Urelumab, e.g., BMS-663513), a CD27 antagonist, an KIR (Killer-cell immunoglobulin-like receptors) antagonist (e.g., an anti-KIR antibody, e.g., Lirilumab or IPH2102/BMS-986015), an IDO (indoleamine 2,3-dioxygenase) inhibitor, a TGF beta (transforming growth factor beta) antagonist, an IL-10 (Interleukin-10) antagonist, a VEGF® (Vascular endothelial growth factor) antagonist, an IL-21 (Interleukin-21) antagonist, a GM-CSF (Granulocyte-macrophage colony-stimulating factor; also known as colony stimulating factor 2 (CSF2)) antagonist, a TLR-9 (Toll-like receptor 9) agonists, and any combination thereof. In certain embodiments, an anti-cancer agent is derived from an anti-tumor vaccine. In other embodiments, an anti-cancer agent is another immune checkpoint inhibitor.

The invention also includes a method of improving or restoring anti-tumor immune response in a subject afflicted with a glioma such that the improved or restored immune cells cross blood-brain barrier in the subject and thus treats the glioma comprising administering an anti-PD-1 antibody or an anti-PD-L1 antibody in a mono-therapy or a combination therapy. In other embodiments, the anti-PD-1 antibody or the anti-PD-L1 antibody crosses the blood brain barrier to treat a glioma.

The present invention shows that when an anti-PD-1 antibody or an anti-PD-L1 antibody is administered to a subject in need thereof, the anti-PD-1 antibody or an anti-PD-L1 antibody is capable of inducing or improving an immune response in the brain of the subject. In one embodiment, administration of the anti-PD-1 antibody or an anti-PD-L1 antibody shows one or more characteristics: (1) increases the number of the immune cells near or in the tumor, (2) increases large aggregation of immune cells near or in the tumor, (3) induces periventricular necrotic tissue near or in the tumor, (4) induces or increases intratumoral macrophages near or in the tumor; (5) induces or increases CD45 positive cells near or in the tumor; (6) induces or increases CD3 positive cells; and (7) induces or increases perivascular cuffing and/or infiltrating cells near or in the tumor. In other embodiments, the present methods are directed to inducing or improving an immune response in the brain of a subject in need thereof comprising administering to the subject a therapeutically effective amount of an anti-PD-1 antibody or an anti-PD-L1 antibody. One or more types of immune responses can be selected from (1) to (7) as described above.

In some embodiments, the present methods provide a method of inducing or improving an immune response in the brain of a subject in need thereof, without directly administering an anti-cancer agent (or an anti-PD-1 antibody or an anti-PD-L1 antibody) to the brain, comprising intravenously administering the anti-PD-1 antibody or an anti-PD-L1 antibody to the subject. The present invention surprisingly shows that the anti-PD-1 antibody or an anti-PD-L1 antibody can induce an immune response in the brain of a subject without directly being administered to the brain. Not being bound by any theory, administration of an anti-PD-1 antibody or an anti-PD-L1 antibody to a subject in need thereof induces or restores an immune response (e.g., activation of immune cells, proliferation of immune cells, increase in the number of immune cells, recruiting of the immune cells to the proper site, etc. . . . ) to the subject, thereby allowing the induced or activated immune cells to cross the blood brain barrier in the subject and eliminate tumor cells. Therefore, administration of an anti-PD-1 antibody or an anti-PD-L1 antibody can (1) increase the number of the immune cells near or in the tumor, (2) increase large aggregation of immune cells near or in the tumor, (3) induce periventricular necrotic tissue near or in the tumor, (4) induce or increase intratumoral macrophages near or in the tumor; (5) induce or increase CD45 positive cells near or in the tumor; (6) induce or increase CD3 positive cells; (7) induce or increase perivascular cuffing and/or infiltrating cells near or in the tumor, or (8) any combination thereof.

In certain embodiments, the method of the present invention (e.g., administration of an anti-PD-1 antibody or an anti-PD-L1 antibody or the administration of an anti-PD-1 antibody or an anti-PD-L1 antibody and another anti-cancer therapy) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 1 year or more when compared to another subject treated with only either another therapy (e.g., Bevacizumab or Temozolomide) or, in the case of combination therapy, only one of the two members of the combination therapy alone (e.g., an anti-PD-1 antibody alone) or an alternative combination therapy (i.e. Temozolomide and radiation therapy). In some embodiments, the duration of survival is increased by at least about 2 months. In certain embodiments, the method of the present invention effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 1 year when compared to another subject treated with only either another therapy (e.g., Bevacizumab or Temozolomide) or, in the case of combination therapy, only one of the two members of the combination therapy alone (e.g., an anti-PD-1 antibody alone) or an alternative combination therapy (i.e. Temozolomide and radiation therapy). In some embodiments, the progression-free survival is increased by at least about 2 months. In certain embodiments, the therapy of the present invention effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another group of subjects treated with only either another therapy (e.g., Bevacizumab or Temozolomide) or, in the case of combination therapy, only one of the two members of the combination therapy alone (e.g., an anti-PD-1 antibody alone) or an alternative combination therapy (i.e. Temozolomide and radiation therapy).

In certain embodiments, the invention is directed to a method of treating newly diagnosed, unmethylated MGMT GBM in a subject in need thereof comprising administering about 240 mg of an anti-PD-1 antibody (i.e., Nivolumab) for about every two weeks for the duration of 16 weeks in combination with focal radiation therapy (2 GY 5×week×6 week). In some embodiments, the method further comprises administering about 480 mg of Nivolumab at 4 weeks after completing the first 16 weeks treatment.

### Anti-PD-1 Antibodies

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

HuMAbs that bind specifically to PD-1 with high affinity have been disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and/or (j) inhibits tumor cell growth in vivo. Anti-PD-1 antibodies usable in the present invention include mAbs that bind specifically to human PD-1 and exhibit at least one, at least two, at least three, at least four or at least five of the preceding characteristics.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody or fragment thereof cross-competes with pembrolizumab. In some embodiments, the anti-PD-1 antibody or fragment thereof binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587; see also http://www.cancer.gov/drugdictionary?cdrid=695789 (last accessed: Dec. 14, 2014). Pembrolizumab has been approved by the FDA for the treatment of relapsed or refractory melanoma.

In other embodiments, the anti-PD-1 antibody or fragment thereof cross-competes with MEDI0608. In still other embodiments, the anti-PD-1 antibody or fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody against the PD-1 receptor. MEDI0608 is described, for example, in U.S. Pat. No. 8,609,089,B2 or in http://www.cancer.gov/drugdictionary?cdrid=756047 (last accessed Dec. 14, 2014).

In certain embodiments, an anti-PD-1 antagonist is AMP-224, which is a B7-DC Fc fusion protein. AMP-224 is discussed in U.S. Publ. No. 2013/0017199 or in http://www.cancer.gov/publications/dictionaries/cancer-drug?cdrid=700595 (last accessed Jul. 8, 2015).

In other embodiments, the anti-PD-1 antibody or fragment thereof cross-competes with BGB-A317. In some embodiments, the anti-PD-1 antibody or fragment thereof binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

Anti-PD-1 antibodies usable in the disclosed methods also include isolated antibodies that bind specifically to human PD-1 and cross-compete for binding to human PD-1 with nivolumab (see, e.g., U.S. Pat. No. 8,008,449; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 as, nivolumab are mAbs. For administration to human subjects, these cross-competing antibodies can be chimeric antibodies, or can be humanized or human antibodies. Such chimeric, humanized or human mAbs can be prepared and isolated by methods well known in the art.

In certain embodiments, the anti-PD-1 antibody or antigen-binding portion thereof comprises a heavy chain constant region which is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen-binding portion thereof is a mAb or an antigen-binding portion thereof. In certain embodiments of any of the therapeutic methods described herein comprising administration of an anti-PD-1 Ab, the anti-PD-1 antibody is nivolumab.

Anti-PD-1 antibodies usable in the methods of the disclosed invention also include antigen-binding portions of the above antibodies. It has been amply demonstrated that the antigen-binding function of an antibody can be performed by fragments of a full-length Ab. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab.

### Anti-PD-L1 Antibodies

In certain embodiments, the present methods are directed to methods of treating a glioma in a subject in need thereof by administering an anti-PD-L1 antagonist (e.g., anti-PD-L1 antibody) to the subject. For example, an anti-PD-L1 antibody prevents interaction between PD-1 and PD-L1, and thus exerts similar effects to the signaling pathway of PD-1; therefore, an anti-PD-L1 antibody can replace the use of an anti-PD-1 antibody in the methods disclosed herein.

In other embodiments, the anti-PD-L1 antibody is BMS-936559 (formerly 12A4 or MDX-1105) (see, e.g., U.S. Pat. No. 7,943,743; WO 2013/173223) or any other anti-PD-L1 antibody disclosed in U.S. Pat. No. 7,943,743. In some embodiments, the anti-PD-L1 antibody is an antibody that cross-competes with BMS-936559 for binding or the anti-PD-L1 antibodies disclosed in U.S. Pat. No. 7,943,743. In other embodiments, the anti-PD-L1 antibody is an antibody that binds to the same epitope as BMS-936559 or the anti-PD-L1 antibody disclosed in U.S. Pat. No. 7,943,743.

In other embodiments, the anti-PD-L1 antibody is MPDL3280A (also known as RG7446) (see, e.g., Herbst; U.S. Pat. No. 8,217,149), MEDI4736 (also called Durvalumab; Khleif, 2013, See U.S. Pat. No. 8,779,108 or US 2014/0356353, filed May 6, 2014), or MSB0010718C (also called Avelumab; See US 2014/0341917). In some embodiments, the anti-PD-L1 antibody is an antibody that cross-competes with MPDL3280A, MEDI4736, and/or MSB0010718C for binding. In embodiments, the anti-PD-L1 antibody is an antibody that binds to the same epitope as MPDL3280A, MEDI4736, and/or MSB0010718C.

In certain embodiments, the antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

Anti-PD-L1 antibodies usable in the methods of the disclosed invention also include antigen-binding portions of the above antibodies. It has been amply demonstrated that the antigen-binding function of an antibody can be performed by fragments of a full-length Ab. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab.

Anti-CTLA-4 antibodies

Anti-CTLA-4 antibodies useful for the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

HuMAbs that bind specifically to CTLA-4 with high affinity have been disclosed in U.S. Pat. Nos. 6,984,720 and 7,605,238. Other anti-CTLA-4 mAbs have been described in, for example, U.S. Pat. Nos. 5,977,318, 6,051,227, 6,682,736, and 7,034,121. The anti-CTLA-4 HuMAbs disclosed in U.S. Pat. Nos. 6,984,720 and 7,605,238 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies usable in the present invention include mAbs that bind specifically to human CTLA-4 and exhibit at least one, at least two, or at least three of the preceding characteristics.

An exemplary clinical anti-CTLA-4 antibody is the human mAb 10D1 (now known as ipilimumab and marketed as YERVOY®) as disclosed in U.S. Pat. No. 6,984,720. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

Another anti-CTLA-4 antibody usable in the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

Anti-CTLA-4 antibodies usable in the disclosed methods also include isolated antibodies that bind specifically to human CTLA-4 and cross-compete for binding to human CTLA-4 with ipilimumab or tremelimumab or bind to the same epitope region of human CTLA-4 as ipilimumab or tremelimumab. In certain embodiments, the antibodies that cross-compete for binding to human CTLA-4 with, or bind to the same epitope region of human CTLA-4 as does ipilimumab or tremelimumab, are antibodies comprising a heavy chain of the human IgG1 isotype. For administration to human subjects, these cross-competing antibodies can be chimeric antibodies, or can be humanized or human antibodies. Usable anti-CTLA-4 antibodies also include antigen-binding portions of the above antibodies such as Fab, F(ab′)2, Fd or Fv fragments.

### Standard-of-Care Therapies for GBM

The present invention also includes a combination therapy of an anti-PD-1 antibody or an anti-PD-L1 antibody with a standard of care therapy for treatment of a glioma. The standard of care therapy can be performed on the subject any time before, during, or after the anti-PD-1 antibody treatment. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014, available at: http://www.nccn.org/professionals/physician_gls/f_guidelines. asp, last accessed May 14, 2014.)

GBM is widely regarded as difficult to treat because the tumors contain many different types of cells and are highly malignant. The first-line treatment for GBM is generally surgery to remove as much of the tumor as possible, and treatment options for recurrent GBM are complicated by side effects and limited efficacy. In one embodiment, the standard of care treatment is MRI-guided laser ablation.

In one embodiment, the present methods include administering an anti-PD-1 antibody or an anti-PD-L1 antibody with bevacizumab. Bevacizumab (AVASTIN®), an anti-vascular growth factor (VEGF) mAb, was approved for the treatment of recurrent GBM in 2009. The approval for bevacizumab for recurrent GBM was based on two single arm clinical trials, AVF3708g and NCI 06-C-0064E. AVF3708g was an open label, multicenter, non-comparative, parallel group study, designed to evaluate the efficacy and safety of bevacizumab monotherapy and bevacizumab plus irinotecan in patients with previously treated GBM. A total of 167 patients were enrolled (85 in the bevacizumab arm, 82 in the bevacizumab plus irinotecan arm). The efficacy of bevacizumab was demonstrated using response assessment based on WHO radiographic criteria. Responses were observed in 25.9% (95% CI:17.0%, 36.1%) of the patients. Median duration of response was 4.2 months (95% CI: 3.0, 5.7 months). The NCI06-C-0064E was a single arm, single site, NCI sponsored study of bevacizumab for patients with previously treated gliomas. A total of 56 patients with high-grade glioma were enrolled in this study. Objective responses observed in this study were 19.6% (95% CI; 10.9%, 31.3%), using the same response criteria as in AVF708g. Median duration of response observed was 3.9 months (95% CI: 2.4, 17.4). (Friedman H. S. et al., J Clin Oncol 27:4733-40 (2009).)

Single agent irinotecan (CPT-11), a topoisomerase 1 inhibitor used in the relapsed setting, produces response rates of ≤15%. In recurrent GBM, PFS-6 rates typically range from 9% to 21%, and median OS is ≤30 weeks. (Friedman H. S. et al., J Clin Oncol 27:4733-40 (2009).)

In higher grade tumors, the treatment options additionally include radiation therapy alone, a combination therapy comprised of radiation and chemotherapy, or a combination therapy comprised of radiation and chemotherapy followed by additional chemotherapy. Therapies for recurrent GBM include repeated neurosurgical resection, radiation therapy, chemotherapy and supportive care. Repeated irradiation becomes more difficult and potential toxicity includes brain necrosis, progressive cerebral edema and persistent neurologic impairment. (Cohen M. E. et al., Pediatr Neurol. 7(3):157-63 (1991). As such, repeated irradiation to the brain is offered to a small minority of patients with recurrent GBM. (Chang S. M. at al. Neurosurg Focus. 20(4):E4 Review (2006))

Nitrosureas, carboplatin, etoposide and irinotecan, or a combination of these agents are common salvage agents administered to recurrent GBM patients. Temozolomide is one form of chemotherapy commonly used to treat GBM, although a multitude of drugs can be used. Additional anti-cancer agents used to treat GBM include: nitrosoureas (e.g., carmustine [BCNU]), MGMT inhibitors (e.g., O6-benzylguanine), cisplatin, bevacizumab (alone or with irinotecan) for recurrent glioma, tyrosine kinase inhibitors (e.g., gefitinib, erlotinib), and other investigational therapies (e.g., gene therapy, peptide and dendritic cell vaccines, synthetic chlorotoxins, radiolabeled drugs and antibodies).

### Pharmaceutical Compositions and Dosages

Therapeutic agents of the present invention can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody is suitable for intravenous, intramuscular, subcutaneous, parenteral, spinal or epidermal administration (e.g., by injection or infusion). A pharmaceutical composition of the invention can include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-PD-1 Ab, including in combination with another anti-cancer agent, the dosage can range from at least about 0.01 to at least about 20 mg/kg, from at least about 0.1 to at least about 10 mg/kg, of the subject's body weight. For example, dosages can be at least about 0.1, at least about 0.3, at least about 1, at least about 2, at least about 3, at least about 5 or at least about 10 mg/kg body weight, and at least about 0.3, at least about 1, at least about 2, at least about 3, or at least about 5 mg/kg body weight. The dosing schedule is typically designed to achieve exposures that result in sustained receptor occupancy (RO) based on typical pharmacokinetic properties of an Ab. An exemplary treatment regime entails administration once per week, once about every 2 weeks, once about every 3 weeks, once about every 4 weeks, once about a month, once about every 3-6 months or longer. In certain embodiments, an anti-PD-1 antibody such as nivolumab is administered to the subject once about every 2 weeks. In other embodiments, the antibody is administered once about every 3 weeks. The dosage and scheduling can change during a course of treatment. For example, a dosing schedule for anti-PD-1 monotherapy can comprise administering the Ab: (i) every 2 weeks in 6-week cycles; (ii) every 4 weeks for six dosages, then every three months; (iii) every 3 weeks; (iv) 3-10 mg/kg once followed by 1 mg/kg every 2-3 weeks. In some embodiments, a dosage regimen for an anti-PD-1 antibody of the invention can comprise about 0.3-10 mg/kg body weight, about 1-5 mg/kg body weight, about 1-3 mg/kg body weight via intravenous administration, with the antibody being given every 14-21 days in up to 6-week or 12-week cycles until complete response or confirmed progressive disease. In some embodiments, the anti-PD-1 antibody is administered at a weight-based dose. In some embodiments, the anti-PD-1 antibody is administered at a flat dose. In some embodiments, the anti-PD-1 antibody is administered at a dose of at least about 60 mg, about 80 mg, about 100 mg, about 120 mg, about 140, about 160 mg, about 180 mg, about 200 mg, about 220 mg, about 240 mg, about 260 mg, about 280 mg, about 300 mg, about 320, about 340, about 360, about 380, about 400, about 420, about 440, about 460, about 480, about 500, or about 550 mg. In some embodiments, the anti-PD-1 antibody is administered at a dose of 240 mg. In other embodiments, the anti-PD-1 antibody is administered at a dose of 480 mg. In some embodiments, the PD-1 antibody is administered in a fixed dose with another antibody. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg PD-1 antibody to mg second antibody.

When used in combinations with other anti-cancer agents, the dosage of an anti-PD-1 antibody can be lowered compared to the monotherapy dose. Dosages of nivolumab that are lower than the typical 3 mg/kg, but not less than 0.001 mg/kg, are subtherapeutic dosages. The subtherapeutic doses of an anti-PD-1 antibody used in the methods herein are higher than 0.001 mg/kg and lower than 3 mg/kg. In some embodiments, a subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%). In some embodiments, 0.3 mg/kg dosing can allow for sufficient exposure to lead to maximal biologic activity.

Ipilimumab is approved for the treatment of melanoma at a dose of at least about 3 mg/kg given intravenously once about every 3 weeks for 4 doses. Thus, in certain embodiments, at least about 3 mg/kg is the highest dosage of ipilimumab used in combination with the anti-PD-1 antibody though, in certain embodiments, an anti-CTLA-4 antibody such as ipilimumab can be dosed within the range of at least about 0.3-10 mg/kg body weight about every two or three weeks when combined with nivolumab. A dosage of ipilimumab that is significantly lower than the approved about 3 mg/kg about every 3 weeks, is regarded as a subtherapeutic dosage The subtherapeutic dosages of an anti-CTLA4 antibody used in the methods herein are higher than 0.001 mg/kg and lower than 3 mg/kg. In some embodiments, the subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight.

In some embodiments, doses of the anti-PD-1 antibody do not exceed about 3 mg/kg when combined with ipilimumab. In certain embodiments, based on risk-benefit and PK-PD assessments, the dosage used comprises a combination of nivolumab at a dose of at least about 1 mg/kg plus ipilimumab at a dose of at least about 3 mg/kg, nivolumab at a dose of at least about 3 mg/kg plus ipilimumab at a dose of at least about 1 mg/kg, or nivolumab at at least about 3 mg/kg plus ipilimumab at a dose of at least about 3 mg/kg is used, each administered at a dosing frequency of once about every 2-4 weeks, once about every 3 weeks. In certain other embodiments, nivolumab is administered at a dosage of at least about 0.1, about 0.3, about 2, about 3 or about 5 mg/kg in combination with ipilimumab administered at a dosage of at least about 0.1, about 0.3, about 1, about 3 or about 5 mg/kg, once about every 2 weeks, once about every 3 weeks, or once about every 4 weeks. In a particular embodiment, nivolumab is administered at a dosage of at least about 3 mg/kg in combination with at least about 1 mg/kg of ipilimumab at a dosing frequency of once about every 3 weeks in four doses.

In certain embodiments, the combination of an anti-PD-1 antibody and an anti-CTLA-4 antibody is administered intravenously to the subject in an induction phase once about every 2 or 3 weeks for 2, 3 or 4 administrations. In certain embodiments, the combination of nivolumab and ipilimumab is administered intravenously in the induction phase about every 3 weeks for 4 administrations. The induction phase is followed by a maintenance phase during which only the anti-PD-1 antibody is administered to the subject at a dosage of at least about 0.1, about 0.3, about 1, about 2, about 3, about 5 or about 10 mg/kg once about every two or three weeks for as long as the treatment proves efficacious or until unmanageable toxicity or disease progression occurs. In certain embodiments, nivolumab is administered during the maintenance phase at a dose of at least about 3 mg/kg body once about every 2 weeks.

For combination of nivolumab with other anti-cancer agents, these agents can be administered at their approved dosages. Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 Ab. The anti-PD-1 antibody can be administered at the dosage that has been shown to produce the highest efficacy as monotherapy in clinical trials, e.g., about 3 mg/kg of nivolumab administered once about every three weeks (Topalian et al., 2012 N Engl J Med 366:2443-54; Topalian et al., 2012 Curr Opin Immunol 24:207-12), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Anti-PD-1 and Anti-PD-L1 Antibodies Suitable for Use in the Disclosed Methods

Anti-PD-1 antibodies suitable for use in the disclosed methods are antibodies that bind to PD-1 with high specificity and affinity, block the binding of PD-L1 and or PD-L2, and inhibit the immunosuppressive effect of the PD-1 signaling pathway. In any of the therapeutic methods disclosed herein, an anti-PD-1 or anti-CTLA-4 “antibody” includes an antigen-binding portion or fragment that binds to the PD-1 or CTLA-4 receptor, respectively, and exhibits the functional properties similar to those of whole antibodies in inhibiting ligand binding and up regulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments for treating a human subject, the antibody is a humanized Ab. In other embodiments for treating a human subject, the antibody is a human Ab. antibodies of an IgG1, IgG2, IgG3 or IgG4 isotype can be used.

Because anti-PD-1 and anti-PD-L1 target the same signaling pathway and have been shown in clinical trials to exhibit similar levels of efficacy in a variety of cancers, (see Brahmer et al., 2012 N Engl J Med 366:2455-65; Topalian et al., 2012a N Engl J Med 366:2443-54; WO 2013/173223), an anti-PD-L1 antibody can be substituted for the anti-PD-1 antibody in any of the therapeutic methods disclosed herein. In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. 2013 J Clin Oncol 31(suppl):3000; U.S. Pat. No. 8,217,149) or MEDI4736 (Khleif, 2013, In: Proceedings from the European Cancer Congress 2013; Sep. 27¬Oct. 1, 2013; Amsterdam, The Netherlands. Abstract 802).

Combination of an Anti-PD-1 Antibody or an Anti-PD-L1 Antibody with an Anti-CTLA-4 Antibody for Treating Gliomas

Also disclosed herein is a method of treating a glioma comprising administering an anti-PD-1 antibody or an anti-PD-L1 antibody and an anti-Cytotoxic T-Lymphocyte Antigen-4 (CTLA-4) antibody, i.e., an antibody or an antigen-binding portion thereof that binds specifically to CTLA-4 and inhibits CTLA-4 activity. The combination of the anti-PD-1 Ab, nivolumab, and the anti-CTLA-4 Ab, ipilimumab, has been demonstrated herein (see Examples 1, 6, 7, and 8) to produce early, durable antitumor activity in glioblastoma patients. Accordingly, in certain embodiments, the anti-CTLA-4 antibody that is used in combination with the anti-PD-1 antibody is ipilimumab. In some embodiments, the anti-CTLA-4 antibody is tremelimumab. In certain other embodiments, the anti-CTLA-4 antibody or antigen-binding portion thereof is a chimeric, humanized or human mAb or a portion thereof. In yet other embodiments, the anti-CTLA-4 antibody or antigen-binding portion thereof comprises a heavy chain constant region which is of a human IgG1 or IgG4 isotype. In some embodiments, the anti-CTLA-4 antibody comprises a heavy chain constant region which is of a human IgG1 isotype.

For the combination of an anti-PD-1 or an anti-PD-L1 antibody and an anti-CTLA-4 Ab, the dosing regimen comprises an induction period (also referred to herein as an induction phase) during which one or more, two or more, three or more, or for or more, combination doses of the anti-PD-1 or the anti-PD-L1 antibody and anti-CTLA-4 Abs are administered to the patient, followed by a maintenance period or phase comprising dosing with the anti-PD-1 antibody or the anti-PD-L1 antibody alone, i.e., not including the anti-CTLA-4 antibody. In certain embodiments, the method comprises (a) an induction phase, wherein the anti-PD-1 or anti-PD-L1 and anti-CTLA-4 antibodies or antigen-binding portions thereof are administered in combination in 2, 4, 6, 8 or 10 doses, each dose ranging from at least about 0.1 to at least about 10.0 mg/kg body weight administered at least once about every 2 weeks, once about every 3 weeks, or once about every 4 weeks, followed by (b) a maintenance phase, wherein no anti-CTLA-4 antibody or antigen-binding portion thereof is administered and the anti-PD-1 antibody or antigen-binding portion thereof or the anti-PD-L1 antibody or antigen-binding portion thereof is repeatedly administered at a dose ranging from about 0.1 to about 10 mg/kg at least once about every 2 weeks, once about every 3 weeks, or once about every 4 weeks.

In certain embodiments, the anti-PD-1 antibody or antigen-binding portion thereof or the anti-PD-L1 antibody or antigen-binding portion thereof is administered at a subtherapeutic dose. In certain other embodiments, the anti-CTLA-4 antibody or antigen-binding portion thereof is administered at a subtherapeutic dose. In further embodiments, both the anti-PD-1 antibody or antigen-binding portion thereof or the anti-PD-L1 antibody or antigen binding portion thereof and the anti-CTLA-4 antibody or antigen-binding portion thereof are each administered at a subtherapeutic dose. In some embodiments anti-PD-1 antibody or antigen-binding portion thereof or the anti-PD-L1 antibody or antigen-binding portion thereof and/or the anti-CTLA-4 antibody or antigen-binding portion thereof is administered at a therapeutic dose.

In certain embodiments, (a) the induction phase comprises at least 4 doses administered at 3-week intervals, wherein the anti-PD-1 and anti-CTLA-4 Abs are administered at the following dosages: (i) at least about 1 mg/kg anti-PD-1 antibody and at least about 3 mg/kg of anti-CTLA-4 Ab; (ii) at least about 3 mg/kg anti-PD-1 antibody and at least about 3 mg/kg of anti-CTLA-4 Ab; (iii) at least about 1 mg/kg anti-PD-1 antibody and at least about 3 mg/kg of anti-CTLA-4 Ab; (iv) at least about 3 mg/kg anti-PD-1 antibody and at least about 3 mg/kg of anti-CTLA-4 Ab; (v) at least about 1 mg/kg anti-PD-1 antibody and at least about 1 mg/kg of anti-CTLA-4 Ab; (vi) at least about 2 mg/kg anti-PD-1 antibody and at least about 3 mg/kg of anti-CTLA-4 Ab; (vii) at least about 3 mg/kg anti-PD-1 antibody and at least about 2 mg/kg of anti-CTLA-4 Ab; (viii) at least about 4 mg/kg anti-PD-1 antibody and at least about 1 mg/kg of anti-CTLA-4 Ab; (ix) at least about 5 mg/kg anti-PD-1 antibody and at least about 1 mg/kg of anti-CTLA-4 Ab; (x) at least about 5 mg/kg anti-PD-1 antibody and at least about 1 mg/kg of anti-CTLA-4 Ab; (xi) at least about 6 mg/kg anti-PD-1 antibody and at least about 1 mg/kg of anti-CTLA-4 Ab; or (xii) at least about 10 mg/kg anti-PD-1 antibody and at least about 1 mg/kg of anti-CTLA-4 Ab, and (b) the maintenance phase comprises repeated administration of the anti-PD-1 antibody at a dose of at least about 3 mg/kg once about every 2 weeks.

Because of durability of the clinical effect previously demonstrated with immunotherapy by inhibition of immune checkpoints (see, e.g., WO 2013/173223), the maintenance phase can include, in alternative embodiments, a finite number of doses, e.g., 1-10 doses, or can involve dosing at long intervals, e.g., once about every 3-6 months or once about every 1-2 years or longer intervals. The maintenance phase can be continued for as long as clinical benefit is observed or until unmanageable toxicity or disease progression occurs.

Given the uncertainty of whether the ipilimumab administered past week 12 contributes to clinical benefit in melanoma and the fact that the U.S. Food and Drug Administration (FDA)- and European Medicines Agency (EMA)-approved schedule for YERVOY® is every 3 weeks for a total of 4 doses, in some embodiments the anti-CTLA-4 antibody is administered during the induction phase once about every 3 weeks for a total of 4 doses. Accordingly, in certain embodiments, the method comprises (a) an induction phase consisting of 4 combination doses administered at 3-week intervals, wherein (i) the anti-PD-1 antibody or antigen-binding portion thereof is administered at a dose of at least about 3 mg/kg body weight and the anti-CTLA-4 antibody or antigen-binding portion thereof is administered at a dose of at least about 1 mg/kg body weight; (ii) the anti-PD-1 antibody or antigen-binding portion thereof is administered at a dose of at least about 1 mg/kg body weight and the anti-CTLA-4 antibody or antigen-binding portion thereof is administered at a dose of at least about 3 mg/kg body weight; (iii) the anti-PD-1 antibody or antigen-binding portion thereof is administered at a dose of at least about 1 mg/kg body weight and the anti-CTLA-4 antibody or antigen-binding portion thereof is administered at a dose of at least about 1 mg/kg body weight; or (iv) the anti-PD-1 antibody or antigen-binding portion thereof is administered at a dose of at least about 3 mg/kg body weight and the anti-CTLA-4 antibody or antigen-binding portion thereof is administered at a dose of at least about 3 mg/kg body weight; and (b) the maintenance phase comprises repeated administration of the anti-PD-1 antibody or antigen-binding portion thereof at a dose of at least about 3 mg/kg once about every 2 weeks. In further embodiments of these methods, the maintenance phase is continued for as long as clinical benefit is observed or until unacceptable or unmanageable toxicity or disease progression occurs.

In a particular embodiment, the method comprises (a) an induction phase consisting of 4 combination doses administered at 3-week intervals, wherein the anti-PD-1 antibody or antigen-binding portion thereof is administered at a dose of at least about 3 mg/kg body weight and the anti-CTLA-4 antibody or antigen-binding portion thereof is administered at a dose of at least about 1 mg/kg body weight; and (b) the maintenance phase comprises repeated administration of the anti-PD-1 antibody or antigen-binding portion thereof at a dose of at least about 3 mg/kg once about every 2 weeks.

Typically, the anti-PD-1 or the anti-PD-L1 and anti-CTLA-4 Abs are formulated for intravenous administration. In other embodiments, the anti-PD-1 antibody or the anti-PD-L1 antibody and/or an anti-CTLA antibody is formulated for direct administration to the central nervous system. In some embodiments, the combination of the anti-PD-1 antibody or an antigen-binding portion thereof or the anti-PD-L1 antibody or an antigen binding portion thereof and the anti-CTLA-4 antibody or an antigen-binding portion thereof is administered concurrently as a single composition or as separate compositions. In some embodiments, the anti-PD-1 or the anti-PD-L1 and anti-CTLA-4 Abs are administered sequentially. In some embodiments, the anti-PD-1 or the anti-PD-L1 and anti-CTLA-4 Abs are administered sequentially during the induction phase. In certain embodiments, when the anti-PD-1 or the anti-PD-L1 and anti-CTLA-4 Abs are administered in combination, they are administered within 30 minutes of each other. Either antibody can be administered first, that is, in certain embodiments, the anti-PD-1 antibody or the anti-PD-L1 antibody is administered before the anti-CTLA-4 Ab, whereas in other embodiments, the anti-CTLA-4 antibody is administered before the anti-PD-1 antibody or the anti-PD-L1 antibody. Typically, each antibody is administered by intravenous infusion over a period of 60 minutes. In certain embodiments, the anti-PD-1 or the anti-PD-L1 and anti-CTLA-4 Abs are administered concurrently, either admixed as a single composition in a pharmaceutically acceptable formulation for concurrent administration, or concurrently as separate compositions with each antibody in a pharmaceutically acceptable formulation.

Certain embodiments of the present methods comprise (a) an induction phase consisting of administration of nivolumab by intravenous infusion followed by administration of ipilimumab by intravenous infusion every 3 weeks for 4 combination doses, followed by (b) maintenance dosing with nivolumab administered by intravenous infusion every 2 weeks starting 3 weeks after the 4th dose of induction therapy or after Day 113 if the 4th dose of induction therapy has not been administered due to treatment delays.

### Kits

Also within the scope of the present invention are kits comprising an anti-PD-1 antibody and another anti-cancer agent for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a glioma, the kit comprising: (a) an amount ranging from at least about 4 mg to at least about 500 mg of an anti-PD-1 antibody or an antigen-binding portion thereof; or (b) an amount of another anti-cancer agent described herein and (c) instructions for using the anti-PD-1 antibody and the other anti-cancer agent in any of the combination therapy methods disclosed herein. In certain embodiments, the anti-PD-1 Ab, the anti-CTLA-4 antibody and/or the other anti-cancer agent can be co-packaged in unit dosage form. In certain embodiments for treating human patients, the kit comprises an anti-human PD-1 antibody disclosed herein, e.g., nivolumab, pembrolizumab, MEDI0608 (formerly AMP-514), AMP-224, or BGB-A317. In other embodiments, the kit comprises an anti-human CTLA-4 antibody disclosed herein, e.g., ipilimumab or tremelimumab.

The present invention is further illustrated by the following examples which should not be construed as further limiting. The contents of all references cited throughout this application are expressly incorporated herein by reference.

### EXAMPLES

### Example 1

Randomized Phase 3 Open Label Study of Nivolumab Versus Bevacizumab and a Safety Study of Nivolumab or Nivolumab in Combination with Ipilimumab in Adult Subjects with Recurrent GBM

The purposes of this study are to: 1) evaluate the safety and tolerability of nivolumab and nivolumab in combination with ipilimumab in subjects diagnosed with recurrent GBM in a safety lead-in group (Cohort 1, 1b); and 2) to evaluate the safety, tolerability, and efficacy of nivolumab versus standard of care treatment (bevacizumab) in subjects diagnosed with recurrent GBM in a randomized trial (Cohort 2).

This is the first study examining monoclonal antibodies targeting immune checkpoint inhibitors in subjects with recurrent GBM. To ensure that nivolumab monotherapy and nivolumab in combination with ipilimumab are tolerable, safety lead-in cohorts (Cohort 1, 1b) evaluating the tolerability of two different treatment regimens were initiated prior to advancing a treatment arm into Cohort 2. Cohort 1 examined in a randomized fashion the safety and tolerability of nivolumab monotherapy dosed at 3 mg/kg every 2 weeks or nivolumab 1 mg/kg+ipilimumab 3 mg/kg every 3 weeks for four doses, then nivolumab 3 mg/kg every 2 weeks thereafter. Cohort 1b examined, in a non-randomized fashion, the safety and tolerability of nivolumab 3 mg/kg+ipilimumab 1 mg/kg every 3 weeks for four doses, then nivolumab 3 mg/kg every 2 weeks thereafter.

Treatment with nivolumab monotherapy in Cohort 1 met the protocol prespecified safety and tolerability profile (less than one-third of subjects required permanent discontinuation due to treatment related adverse events prior to receiving 4 doses of treatment) to advance to Cohort 2, the randomized portion of the study to compare nivolumab monotherapy versus bevacizumab. Since evaluation of a dosing regimen for the combination therapy is on-going in Cohort 1 and 1b, Cohort 2 was designed to evaluate nivolumab monotherapy versus bevacizumab. Evaluation of the efficacy of nivolumab+ipilimumab combination therapy will be evaluated separately, once there is sufficient data from Cohort 1 and Cohort 1b to select the appropriate combination dosing to advance into a randomized study.

Nivolumab dosing in Cohort 2 is based upon preliminary clinical experience in subjects with recurrent GBM from Cohort 1. Subjects in the nivolumab arm will receive 3 mg/kg every 2 weeks, and subjects in the bevacizumab arm will be dosed at 10 mg/kg every 2 weeks. Subjects will continue to receive study medication until confirmed tumor progression, unacceptable toxicity, or other discontinuation criteria described herein, whichever came first. Upon discontinuation of treatment, subjects will enter a follow-up phase to gather information on overall survival.

This study will also provide data regarding progression free survival, objective response rate, biomarkers of disease progression, and health outcomes. Subjects in the combination arms who discontinued nivolumab+ipilimumab due to treatment related adverse events prior to completing the fourth dose may begin nivolumab 3 mg/kg every 2 weeks with prior approval of the Sponsor.

### Methods

### Study Design

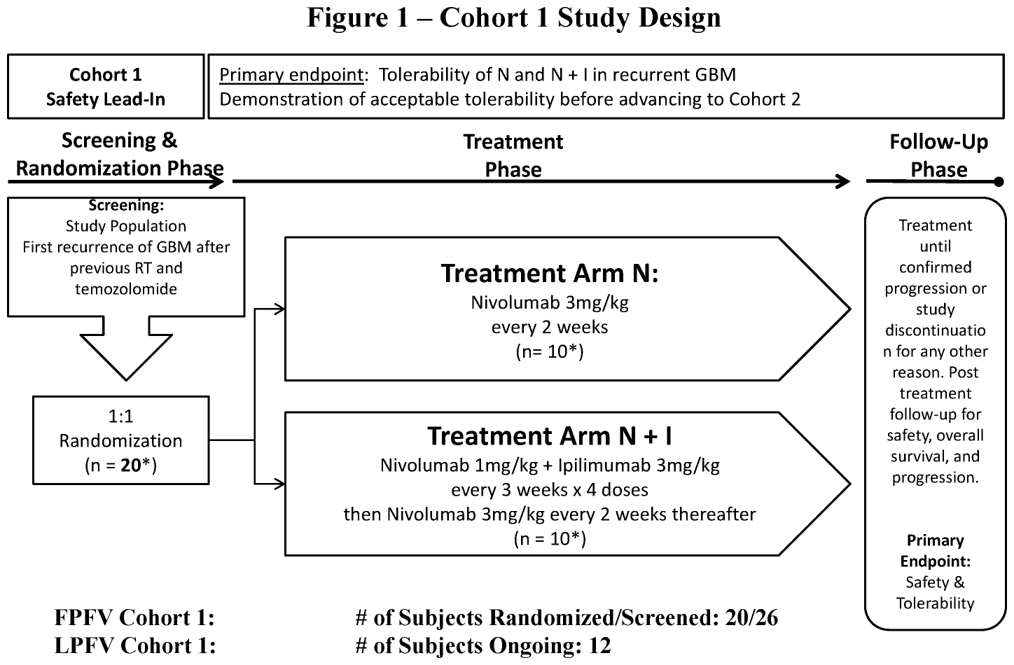

A randomized, open-label, multicenter, phase 3 study of nivolumab monotherapy versus bevacizumab and a safety study of nivolumab and nivolumab in combination with ipilimumab is conducted in adult (>18 years) subjects with a first recurrence of GBM after treatment with radiotherapy and temozolomide. Since the use of nivolumab and ipilimumab has not previously been studied in subjects with GBM, a safety lead-in (Cohort 1) consisting of approximately 10 subjects in each of two treatment arms are enrolled and randomized to treatment with either Arm N (nivolumab monotherapy) or Arm N+I (nivolumab combined with ipilimumab). Tolerability and safety of a treatment arm is determined after all subjects in the arm completed four doses or discontinued dosing prior to completing four doses. A safety evaluation of Cohort 1 is conducted for each arm based on criteria described herein. The study design for Cohort 1 is summarized in FIG. 1.

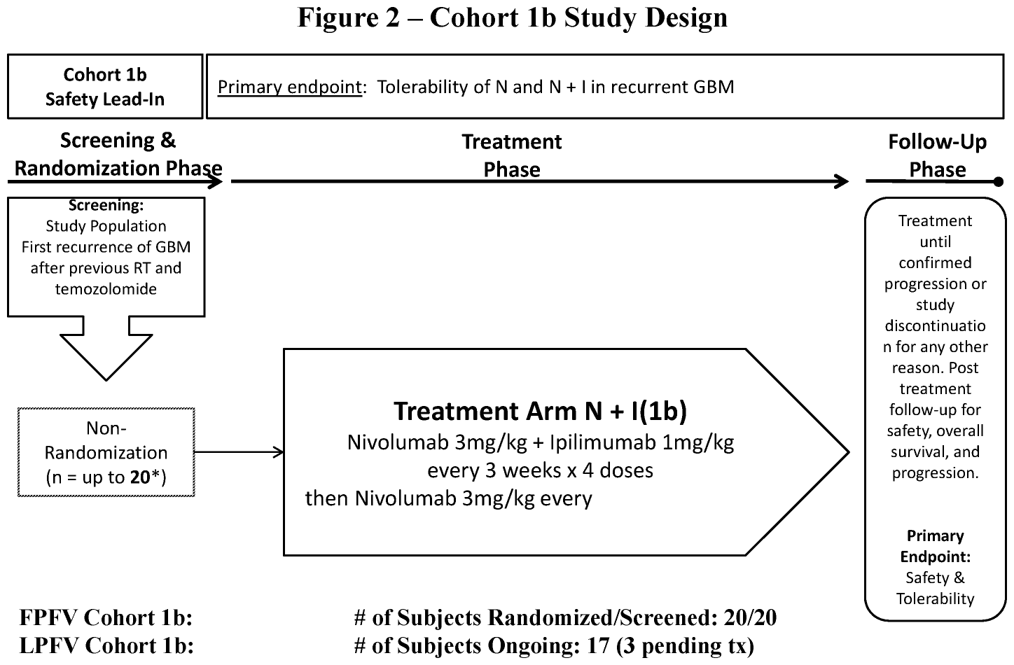

In order to better characterize preliminary safety and tolerability for the combination therapy, an additional, non-randomized safety cohort (Cohort 1b) to evaluate an alternate dosing regimen is initiated for the combination therapy (nivolumab 3 mg/kg+ipilimumab 1 mg/kg every 3 weeks for 4 cycles followed by nivolumab 3 mg/kg thereafter). The study design for Cohort 1b is summarized in FIG. 2. Evaluation of the efficacy of nivolumab+ipilimumab combination therapy will be evaluated separately. Information from subjects treated with combination therapy in Cohort 1 and Cohort 1b will provide a basis for dose selection for future studies in subjects with GBM.

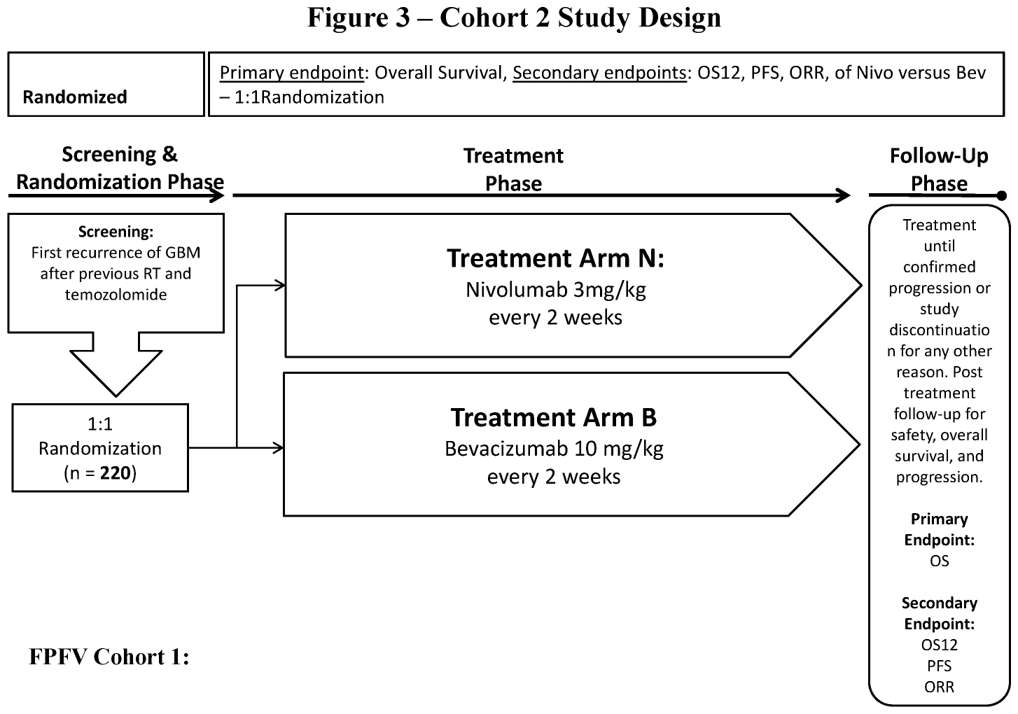

Enrollment in Cohort 2 is planned to begin after safety and tolerability of the safety lead-in has been evaluated and determination regarding which treatments arms (Arm N and/or Arm N+I) will be studied. Treatment with nivolumab monotherapy in Cohort 1 met the protocol prespecified safety and tolerability profile (less than one-third of subjects required permanent discontinuation due to treatment related adverse events prior to receiving 4 doses of treatment) to advance to Cohort 2, the randomized portion of the study to compare nivolumab monotherapy versus bevacizumab. The study design for Cohort 2 is summarized in FIG. 3. Since evaluation of a second dosing regimen for the combination therapy is on-going, the randomized portion of this study (Cohort 2) is limited to nivolumab monotherapy versus bevacizumab. Evaluation of the efficacy of nivolumab+ipilimumab combination therapy will be evaluated separately, once there is sufficient data from Cohort 1 and 1b to select the appropriate dose to advance (either nivolumab 3 mg/kg+ipilimumab 1 mg/kg or nivolumab 1 mg/kg+ipilimumab 3 mg/kg) into a randomized study.

All subjects in Cohort 1, 1b, and 2 will continue to be followed for safety and tolerability, tumor progression, and overall survival. Tumor progression or response endpoints are assessed using Radiologic Assessment in Neuro-Oncology criteria (RANO) as described herein. Treatment with study medication continued until confirmed tumor progression, unacceptable toxicity, or other discontinuation criteria as described herein, whichever came first.

### Study Phases

This study comprises three phases: screening, treatment (Cohort 1, 1b, and Cohort 2), and follow-up.

Screening Phase—The screening phase begins by establishing the subject's initial eligibility and signing of the informed consent form (ICF). The subject is enrolled using the Interactive Voice Response System (IVRS).

Treatment Phase—The treatment phase begins with the randomization call to the IVRS. Subjects in Cohort 1 and Cohort 2 are randomly assigned to one of the treatment arms based on the study cohort. Subjects in Cohort 1b are assigned to Arm N+I_1b. Within 3 working days from treatment assignment, the subject will receive the first dose of study medication. Arm N (nivolumab monotherapy) will receive nivolumab 3 mg/kg IV every two weeks. Arm N+I (nivolumab+ipilimumab) will receive nivolumab 1 mg/kg IV combined with ipilimumab 3 mg/kg IV every three weeks for 4 doses, then nivolumab 3 mg/kg IV every two weeks (Cohort 1 only). Arm N+I_1b (nivolumab+ipilimumab) will receive nivolumab 3 mg/kg IV combined with ipilimumab 1 mg/kg IV every three weeks for 4 doses, then nivolumab 3 mg/kg IV every two weeks (Cohort 1b only). Arm B (bevacizumab) Cohort 2 will receive bevacizumab 10 mg/kg every 2 weeks. Adverse event assessments are documented at each visit throughout the study. Quality of Life assessments using EORTC QLQ-C30 and BNS20 and EQ-5D are completed on Day 1 Week 1.

All of the laboratory tests and vital signs are collected prior to study drug dosing, including, but not limited to, CBC with differential, LDH, AST, ALT, ALP, T.Bili, BUN or serum urea level, creatinine, Ca, Mg, Na, K, Cl, Glucose, amylase, lipase, TSH with reflexive Free T4, and Free T3. PK, immunogenicity, and biomarker blood samples are also collected. Study drug dosing is delayed in some cases for toxicity. Treated subjects are evaluated for response by the investigator and according to the RANO criteria. Tumor assessments are performed beginning at the end of Week 6 (±1 week) after first dose, at the end of Week 12 (±1 week), and continuing every 8 weeks (±1 week) until disease progression or treatment discontinuation, whichever occurs later. Treated subjects are evaluated for neurologic functioning by the investigator and according to the NANO scale (Cohort 2 only). Assessments are performed beginning at the end of Week 6 (±1 week) after first dose, at the end of Week 12 (±1 week), and continuing every 8 weeks (±1 week) until disease progression or treatment discontinuation, whichever occurs later. Treated subjects are evaluated for neurocognitive functioning by the investigator and according Cogstate assessment (Cohort 2 only). Assessments are performed at Screening, Week 1, Week 13, Week 21, Week 45, Week 69, and treatment discontinuation (if >4 weeks from prior Cogstate assessment).

The treatment phase ends when the subject experiences a confirmed tumor progression, unacceptable toxicity, or other discontinuation criteria, whichever occurs first.

Follow-up Phase—The follow-up phase begins when the decision to discontinue a subject from study therapy is made (no further treatment with study therapy). Two follow-up visits includes collection of PK/immunogenicity samples, and Quality of Life questionnaires are completed. Subjects who discontinued treatment for reasons other than tumor progression continue to have tumor assessments beginning at the end of Week 6 (±1 week) after first dose, at the end of Week 12 (±1 week) and then every 8 (±1 week) weeks until disease progression or withdrawal of consent. All radiologically determined disease progression is confirmed by an additional confirmatory MRI scan approximately 12 weeks following the initial assessment of radiological progression. Investigators obtains additional follow-up MRI scans prior to 12 weeks as medically appropriate. Subjects will be followed for drug-related toxicities until these toxicities resolved, returned to baseline, or are deemed irreversible. All adverse events will be documented for a minimum of 100 days after last dose. After completion of the first two follow-up visits, subjects will be followed every 3 months for survival.

### Study Population

The study population includes subjects >18 years old with histologically confirmed diagnosis of WHO Grade IV malignant glioma (GBM or gliosarcoma). The population includes subjects previously received first line treatment with at least radiotherapy and temozolomide. Subjects who have a documented first recurrence of GBM by diagnostic biopsy or contrast enhanced MRI performed within 21 days of randomixation per RANO criteria are selected as study population.

If first recurrence of GBM is documented by MRI, an interval of at least 12 weeks after the end of prior radiation therapy is required unless there is either: i) histopathologic confirmation of recurrent tumor, or ii) new enhancement on MRI outside of the radiotherapy treatment field. All subjects are required to wait at least 28 days and to exhibit a full recovery (i.e., no ongoing safety issues) following surgical resection prior to randomization. Subjects are to wait at least 4 weeks after the last administration of any other treatment for GBM. Subjects are required to have a Karnofsky performance status of 70 or higher and a life expectancy of at least 12 weeks.

To participate in Cohort 1 or 1b, subjects are required to have at least one measurable GBM lesion prior to randomization that meets the following criteria: i) contrast enhancing and clearly defined, bi-dimensionally measurable margins, and ii) at least two perpendicular diameters measuring >10 mm by >10 mm. MRI measurements do not include surgical cavity, cyst, or necrotic area.

Subjects are prohibited from concomitant treatment with any drug or other investigational agents for treatment of GBM (i.e., chemotherapy, hormonal therapy, immunotherapy, radiation therapy) and any medications contraindicated with bevacizumab treatment.

Subjects are excluded from this study if they had more than one recurrence of GBM, have extracranial metastatic or leptomeningeal disease, or have been diagnosed with secondary GBM (i.e., GBMs that progress from low grade diffuse astrocytoma or anaplastic astrocytoma). Subjects testing positive for hepatitis B virus surface antigen (HBV sAg), detectable hepatitis C virus ribonucleic acid (HCV RNA) indicating acute or chronic infection, or with a known history of testing positive for human immunodeficiency virus (HIV) or acquired immunodeficiency syndrome (AIDS) are also excluded from this study.

In addition, subjects are excluded for any of the following reasons:
a) Any serious or uncontrolled medical disorder that, in the opinion of the investigator, may increase the risk associated with study participation or study drug administration, impair the ability of the subject to receive protocol therapy, or interfere with the interpretation of study results.
b) Subjects with active, known or suspected autoimmune disease. Subjects with vitiligo, type I diabetes mellitus, residual hypothyroidism due to autoimmune condition only requiring hormone replacement, psoriasis not requiring chronic and systemic immunosuppressive treatment, or conditions not expected to recur in the absence of an external trigger were permitted to enroll;
c) Previous radiation therapy with anything other than standard radiation therapy (i.e., focally directed radiation);
d) Subjects requiring escalating or chronic supraphysiologic doses of corticosteroids for control of their disease at randomization are excluded;
e) Previous treatment with carmustine wafer except when administered as first line treatment and at least 6 months prior to randomization;
f) Previous bevacizumab or other VEGF or anti-angiogenic treatment (Cohort 2 only);
g) Previous treatment with a PD-1 or CTLA-4 targeted therapy;
h) Evidence of >Grade 1 CNS hemorrhage on the baseline MRI scan;
i) Inadequately controlled hypertension (defined as systolic blood pressure >150 mmHg and/or diastolic blood pressure >100 mmHg) within 28 days of first study treatment;
j) Prior history of hypertensive crisis, hypertensive encephalopathy, reversible posterior leukoencephalopathy syndrome (RPLS);
k) Prior history of gastrointestinal diverticulitis, perforation, or abscess;
l) Clinically significant (i.e., active) cardiovascular disease, for example cerebrovascular accidents <6 months prior to study enrollment, myocardial infarction <6 months prior to study enrollment, unstable angina, New York Heart Association (NYHA) Grade II or greater congestive heart failure (CHF), or serious cardiac arrhythmia uncontrolled by medication or potentially interfering with protocol treatment;
m) History or evidence upon physical/neurological examination of central nervous system disease (e.g., seizures) unrelated to cancer unless adequately controlled by medication or potentially interfering with protocol treatment;
n) Significant vascular disease (e.g., aortic aneurysm requiring surgical repair or recent arterial thrombosis) within 6 months prior to start of study treatment. Any previous venous thromboembolism >NCI CTCAE Grade 3;
o) History of pulmonary hemorrhage/hemoptysis >grade 2 (defined as >2.5 mL bright red blood per episode) within 1 month prior to randomization;
p) History or evidence of inherited bleeding diathesis or significant coagulopathy at risk of bleeding (i.e., in the absence of therapeutic anticoagulation);
q) Current or recent (within 10 days of study enrollment) use of anticoagulants that, in the opinion of the investigator, would place the subject at significant risk for bleeding. Prophylactic use of anticoagulants was allowed;
r) Surgical procedure (including open biopsy, surgical resection, wound revision, or any other major surgery involving entry into a body cavity) or significant traumatic injury within 28 days prior to first study treatment, or anticipation of need for major surgical procedure during the course of the study;
s) Minor surgical procedure (e.g., stereotactic biopsy within 7 days of first study treatment; placement of a vascular access device within 2 days of first study treatment);
t) History of intracranial abscess within 6 months prior to randomization;
u) History of active gastrointestinal bleeding within 6 months prior to randomization;
v) Serious, non-healing wound, active ulcer, or untreated bone fracture; and/or
w) Subjects unable (due to existent medical condition, e.g., pacemaker or ICD device) or unwilling to have a head contrast enhanced MRI.

### Discontinuation Criteria

Treatment with nivolumab and/or ipilimumab must be permanently discontinued if any Grade 2 drug-related uveitis or eye pain or blurred vision that does not respond to topical therapy and does not improve to Grade 1 severity within the re-treatment period OR requires systemic treatment. The treatment will also be discontinued if any Grade 3 non-skin, drug-related adverse event lasting >7 days, with the following exceptions for drug-related laboratory abnormalities, uveitis, pneumonitis, bronchospasm, diarrhea, colitis, neurologic toxicity, hypersensitivity reactions, and infusion reactions:
(a) Grade 3 drug-related uveitis, pneumonitis, bronchospasm, diarrhea, colitis, neurologic toxicity, hypersensitivity reaction, or infusion reaction of any duration requires discontinuation.
(b) Grade 3 drug-related laboratory abnormalities do not require treatment discontinuation except:
   (i) Grade 3 drug-related thrombocytopenia >7 days or associated with bleeding requires discontinuation
   (ii) Any drug-related liver function test (LFT) abnormality that meets the following criteria require discontinuation:
      AST or ALT>8×ULN
      Total bilirubin >5×ULN
      Concurrent AST or ALT>3×ULN and total bilirubin >2×ULN

The treatment can also be discontinued if any Grade 4 drug-related adverse event or laboratory abnormality, except for the following events which do not require discontinuation:
(a) Grade 4 amylase or lipase abnormalities that are not associated with symptoms or clinical manifestations of pancreatitis. It is recommended to consult with the BMS Medical Monitor for Grade 4 amylase or lipase abnormalities.
(b) Isolated Grade 4 electrolyte imbalances/abnormalities that are not associated with clinical sequelae and are corrected with supplementation/appropriate management within 72 hours of their onset

The treatment will be discontinued if any dosing interruption lasting >6 weeks from the last dose with the following exceptions:
(a) Dosing interruptions to allow for prolonged steroid tapers to manage drug-related adverse events are allowed. Prior to re-initiating treatment in a subject with a dosing interruption lasting >6 weeks, the BMS medical monitor must be consulted. Tumor assessments should continue as per protocol even if dosing is interrupted.
(b) Dosing interruptions >6 weeks from the last dose that occur for non-drug-related reasons may be allowed if approved by the BMS medical monitor. Prior to re-initiating treatment in a subject with a dosing interruption lasting >6 weeks, the BMS medical monitor must be consulted. Tumor assessments should continue as per protocol even if dosing is interrupted.

The treatment will be discontinued if any adverse event, laboratory abnormality, or intercurrent illness which, in the judgment of the Investigator, presents a substantial clinical risk to the subject with continued nivolumab or ipilimumab dosing.

In addition, treatment with bevacizumab must be permanently discontinued according to the updated bevacizumab package insert.

The treatment will also be discontinued if the subjects display grade 3 or grade 4 symptoms (e.g., Severe reaction). Grade 3 symptoms include prolonged symptoms (i.e., not rapidly responsive to symptomatic medication and/or brief interruption of infusion); recurrence of symptoms following initial improvement; or hospitalization indicated for other clinical sequelae (e.g., renal impairment, pulmonary infiltrates)). Grade 4 includes life-threatening symptoms or pressor or ventilatory support indicated.

Study treatment must be discontinued upon confirmed radiological progression or clinical progression, whichever occurs first. Details of the radiological progression or clinical progression are described below:

Standard treatment for GBMs (including radiation therapy and temozolomide) may result in a transient increase in tumor enhancement (pseudoprogression) in a subset of subjects that eventually subsides without any change in therapy. Pseudoprogression may be difficult to differentiate from true tumor progression and may have important implications for patient management. Some evidence also indicates that some subjects treated with immune system stimulating agents may also develop apparent progression of disease (by conventional response criteria) before demonstrating clinical objective responses and/or stable disease. This phenomenon was observed in approximately 10% of subjects in the Phase 1 study of nivolumab and has also been reported for ipilimumab monotherapy.

In order to minimize premature discontinuation of study medication and distinguish pseudoprogression from progressive disease, subjects initially meeting radiologic criteria for disease progression may continue receiving study medication until confirmation of progression with an MRI performed approximately 12 weeks later.

In order to continue study treatment after assessment of initial radiological progression, the following criteria must be met:
(a) The subject is believed to demonstrate clinical benefit as determined by the investigator.
(b) The subject is tolerating study medication.

Subjects with confirmed progression (approximately 12 weeks after initially assessed progression) will discontinue study medication and enter the follow up/survival phase of the study. If progression is confirmed then the date of disease progression will be the first date the subject met the criteria for progression.

In those cases in which radiologic progression cannot be differentiated from pseudoprogression and it is the investigator's opinion that a surgical resection to obtain tumor tissue for histopathology is in the subject's best interests, a surgical resection may be performed following consultation with the BMS medical monitor. Tumor biopsy samples (blocks or slides) must be submitted for central review by a neuropathologist to minimize any inter-observer variation in the histopathologic assessment of progression versus treatment-related changes. If tumor pathology confirms progression, then the subject will be discontinued from study medication per protocol discontinuation criteria and the date of progression will be the day that it was first suspected. If tumor pathology reveals treatment-related changes and does not confirm disease progression, the subject may continue study medication. An MRI after the resection is required prior to treatment continuation. The subject will then continue all on-treatment tumor assessments as per the treatment schedule.

In those cases in which radiologic progression cannot be differentiated from pseudoprogression and the subject undergoes a biopsy or diagnostic surgical resection to obtain tumor tissue for histopathology analysis, representative tumor tissue samples will be reviewed locally and submitted for central review by a neuropathologist. If there is discordance between the central and local neuropathologic determination of progression versus treatment-associated changes (pseudoprogression), then a second central neuropathologic review will be performed and serve as the final adjudication of the histopathologic determination. Central review of histopathology will be blinded to the subject treatment arm assignment. Specific instructions will be provided to the investigative sites regarding the requirements for submission of tumor tissue for central neuropathologic review.

### Dosing Information

The dosing regimen and schedule for Arm N (nivolumab) and Arms N+I (nivolumab and ipilimumab) and Arm B (bevacizumab) are detailed in Table 1, Table 2, Table 3 and Table 4. When nivolumab and ipilimumab are administered on the same day, separate infusion bags and filters are used for each infusion. Nivolumab is administered first. The second infusion is always ipilimumab, and it starts no sooner than 30 minutes after completion of the nivolumab infusion.

TABLE 1
Dosing Schedule for Arm N Nivolumab Monotherapy

Every 2 Week Dosing

| | |
|---|---|
| Day 1, Week 1 | Day 1, Week 3 and every two weeks thereafter |
| 3 mg/kg Nivolumab | 3 mg/kg Nivolumab |

TABLE 2
Dosing Schedule for Arm N + 1 (Week
1-12) Nivolumab and Ipilimumab Combination

| Every 3 Week Dosing | |
|---|---|
| Day 1 | |
| Week 1, Week 4, Week 7, Week 10 | |
| Cohort 1 | Cohort 1b |
| 1 mg/kg Nivolumab | 3 mg/kg Nivolumab |
| 3 mg/kg Ipilimumab | 1 mg/kg Ipilimumab |

TABLE 3
Dosing Schedule for Arm N + 1 (Week 13 and following)
Nivolumab + Ipilimumab Combination

Every 2 Week Dosing
Day 1
Week 13 and every other week thereafter
3 mg/kg Nivolumab

TABLE 4
Dosing Schedule for Arm B (Bevacizumab monotherapy)

| Every 2 Week Dosing | |
|---|---|
| Day 1 | Day 1 |
| Week 1 | Week 3 and every two weeks thereafter |
| 10 mg/kg | 10 mg/kg |

Dosing Window Arm N+I (Week 1-12)

Subjects are dosed no less than 19 days between doses and no more than 3 days after the scheduled dosing date. Dose given after the 3 day window is considered a dose delay. A maximum delay of 42 days between doses is allowed. If dosing is delayed, both nivolumab and ipilimumab are delayed together. If dosing is resumed after a delay, both nivolumab and ipilimumab are resumed on the same day.

### Dosing Window Arm N, Arm B and Arm N+I (Week 13 and Following)

Subjects are dosed no less than 12 days between doses and no more than 3 days after the scheduled dosing date. Dose given after the 3 day window is considered a dose delay. A maximum delay of 42 days between doses is allowed.

Study assessments and procedures are described in Table 5. In particular, screening assessments are described in Table 5A; on-study assessments Nivolumab (N) and Bevacizumab (B) Arms are shown in Table 5B; on-study assessments Nivolumab+Ipilimumab (N+I) Arm are described in Table 5C; and follow-up assessments are described in Table 5D.

### Efficacy Assessment

Head MRI

All subjects received efficacy assessments with brain MRI with contrast on Day 1 Week 7, Day 1 Week 13, and then one time every 8 weeks (±1 week). MRIs occurred at ±7 days per scheduled visits. Investigators will obtain more frequent follow-up MRI scans as medically indicated.

Local radiologic assessment of tumor measurements were used during the study for clinical management and investigator-assessed disease progression. Cases of suspected radiologic disease progression were confirmed by an MRI performed approximately 12 weeks after the initial radiological assessment of progression. Safety Assessment

At baseline, a medical history will be obtained to capture relevant underlying conditions. The baseline examinations include weight, height, Karnofsky Performance Status, BP, HR, temperature, 12-lead ECG and oxygen saturation by pulse oximetry at rest and after exertion. Baseline signs and symptoms are those that are assessed within 14 days prior to randomization. Concomitant medications will be collected from within 14 days prior to randomization through the study treatment period.

Baseline local laboratory assessments are to be done within 14 days prior to randomization to include: CBC w/differential, ANC, platelets, Hgb; Chemistry panel including ALT, AST, total bilirubin, alkaline phosphatase), BUN or serum urea level, creatinine, Ca, Mg, Na, K, Cl, LDH, glucose, amylase, lipase, TSH, Free T4, Free T3, and Hep B and C testing (HBV sAg, HCV antibody or HCV RNA). Pregnancy testing for WOCBP (done locally) must be performed within 24 hours prior to the initial administration of study drug at baseline. Where required by local regulations, an HIV test must also be performed.

Subjects will be evaluated for safety if they have received any study drug. Toxicity assessments will be continuous during the treatment phase and the safety follow-up phase. Adverse events and laboratory values will be graded according to the NCI-CTCAE version 4.0.

On-study weight, Karnofsky performance status, and vital signs will be assessed at each on-study visit prior to dosing. Vital signs will also be taken as per institutional standard of care prior to, during and after dosing. Oxygen saturation by pulse oximetry at rest and after exertion will be assessed at each on study visit prior to dosing. Urinalysis (dipstick) will be obtained within 72 hours prior to dose for subjects randomized to bevacizumab. The start and stop time of the nivolumab and the ipilimumab infusion and bevacizumab administration will be documented. Physical examinations are to be performed as clinically indicated. If there are any new or worsening clinically significant changes since the last exam, report changes on the appropriate non-serious or serious adverse events page.

Additional measures, including non-study required laboratory tests, will be performed as clinically indicated or to comply with local regulations. On-treatment 12-lead ECGs will be obtained if clinically indicated. Laboratory toxicities (e.g., suspected drug inducted liver enzyme elevations) will be monitored during the follow-up phase via on site/local labs until all study drug related toxicities resolve, return to baseline or are deemed irreversible.

Oxygen saturation by pulse oximetry will be obtained prior to each dose of nivolumab and at any time a subject has any new or worsening respiratory symptoms. A reading at rest will be obtained at each time point. If a subject shows changes on pulse oximetry or other pulmonary related signs (e.g., hypoxia, fever) or symptoms (e.g., dyspnea, cough) consistent with possible pulmonary adverse events, the subject will be immediately evaluated to rule out pulmonary toxicity.

Urinalysis should be obtained for all subjects within 72 hours prior to dose. Urinalysis will be collected on treatment only for subjects randomized to bevacizumab. Subjects with 2+ or greater protein urea urine dipstick reading must undergo further assessment with a 24 hour collection prior to being dosed. Bevacizumab must be held for ≥2 grams of proteinuria/24 hours and may resume when proteinuria is <2 gm/24 hours. Treatment should be discontinued in subjects with nephrotic syndrome.

Some of the previously referred to assessments may not be captured as data in the eCRF. They are intended to be used as safety monitoring by the treating physician. Additional testing or assessments may be performed as clinically necessary or where required by institutional or local regulations.

### Imaging Assessment for the Study

All subjects will receive safety, tumor measurements and volumetric Magnetic Resonance Imaging (MRI) of the head within 21 days prior to randomization. See Table 5A. For Arm N and Arm B (Cohort 2), contrast-enhanced MRI will be performed weeks 7 and 13, day 1 and then every 8 weeks (+/−1 week). See Table 5B. For Arm N+I, contrast-enhanced MRI was performed at day 1 of Weeks 7 and 13 and then every 8 weeks (+/−1 week). See Table 5C. For follow-up assessments in all subjects, tumor assessment was required for subjects who did not progress while on study therapy, including subjects who start subsequence anti-cancer therapy. In the follow up assessment, the tumor assessment is obtained at the end of Weeks 6 and 12 (+/−1) and then every 8 weeks (+/−1 week thereafter. See Table 5D.

Investigators may obtain more frequent follow-up MRI scans as medically indicated. All radiologic imaging from this study will be transmitted to a centralized imaging core lab for storage and may be reviewed by an Independent Radiology Review Committee (IRRC) as determined by the Sponsor. Any incidental findings of potential clinical relevance that are not directly associated with the objectives of the protocol should be evaluated and handled by the Study Investigator as per standard medical/clinical judgment. Study sites will retain local access to the imaging results for safety and efficacy reading purposes. The study investigator will review the local MRI results as clinically appropriate to ensure that any potentially emergent clinical situations are addressed in a timely fashion.

Clinically significant radiologic findings or changes from baseline scans will be coded as adverse events or serious adverse events.

### Efficacy Assessments

The primary efficacy measure of the study is overall survival (OS). OS is defined as the time between the date of randomization and the date of death due to any cause. OS is followed continuously while subjects are on study drug and every 3 months via in-person or phone contact during the survival follow-up phase of the study.

Investigator-assessed tumor response is based upon published RANO criteria. Tumor assessments are performed as shown above in the imaging assessment for the study. Radiologic response was assessed by comparing the pretreatment baseline and on-treatment MRI scans. Radiologic progression was determined by using the smallest tumor measurement at either the pretreatment baseline or after initiation of study medication.

A complete response required: complete disappearance of all enhancing measurable and nonmeasurable disease sustained for at least 4 weeks; no new lesions; stable or improved nonenhancing (T2/FLAIR) lesions; patients must be off corticosteroids (or on physiologic replacement doses only); and stable or improved clinically. Patients with nonmeasurable disease only cannot have a complete response; the best response possible is stable disease.

A partial response required: >50% decrease compared with baseline in the sum of products of perpendicular diameters of all measurable enhancing lesions sustained for at least 4 weeks; no progression of nonmeasurable disease; no new lesions; stable or improved nonenhancing (T2/FLAIR) lesions on same or lower dose of corticosteroids compared with baseline scan; the corticosteroid dose at the time of the scan evaluation should be no greater than the dose at time of baseline scan; and stable or improved clinically. Patients with nonmeasurable disease only cannot have a partial response; the best response possible is stable disease.

A stable disease required: does not qualify for complete response, partial response, or progression; and stable nonenhancing (T2/FLAIR) lesions on same or lower dose of corticosteroids compared with baseline scan. In the event that the corticosteroid dose is increased for new symptoms and signs without confirmation of disease progression on neuroimaging, and subsequent follow-up imaging shows that this increase in corticosteroids was required because of disease progression, the last scan considered to show stable disease will be the scan obtained when the corticosteroid dose was equivalent to the baseline dose.

Progression is defined by any of the following: >25% increase in sum of the products of perpendicular diameters of enhancing lesions compared with the smallest tumor measurement obtained either at baseline (if no decrease) or best response, on stable (including subjects not on corticosteroids) or increasing doses of corticosteroids; significant increase in T2/FLAIR nonenhancing lesions on stable or increasing doses of corticosteroids compared with baseline scan or best response after initiation of therapy not cause by comorbid events (e.g., radiation therapy, demyelination, ischemic injury, infection, seizures, postoperative changes, or other treatment effects); any new lesion; clear clinical deterioration not attributable to other causes apart from the tumor (e.g., seizures, medication adverse effects, complications of therapy, cerebrovascular events, infection, and so on) or changes in corticosteroid dose; failure to return for evaluation as a result of death or deteriorating condition; or clear progression of nonmeasurable disease.

TABLE 5
Flow Chart/Time and Events Schedule
Table 5A. Screening Assessments (CA209143)

| | | |
|---|---|---|
| | Screening | |
| | Visit: within 28 days of | |
| Procedure | randomization | Notes |

Eligibility Assessments

| | | |
|---|---|---|
| Informed Consent | X | Contact IVRS to obtain study subject number. Study allows |
| | | for re-enrollment of a subject that has discontinued the |
| | | study as a pre-treatment failure. If re-enrolled, the |
| | | subject must be re-consented and assigned a new subject |
| | | number from IVRS |
| Inclusion/Exclusion Criteria | X | All inclusion/exclusion criteria should be assessed at |
| | | screening and confirmed prior to randomization |

Disease Status

| | | |
|---|---|---|
| Medical History | X | |
| Gene methylation status | | If available |
| Tumor Biopsy | X | Archival or fresh biopsy taken at any point prior to |
| | | study treatment. Submit to central vendor. Medical |
| | | monitor approval is required for subjects for whom |
| | | tissue is not available.; |

Safety Assessments

| | | |
|---|---|---|
| Physical Examination | X | |
| Vital signs and oxygen saturation | X | Including BP, HR, temperature and oxygen saturation |
| | | by pulse oximetry. Pulse oximetry at rest and after |
| | | exertion. Obtain vital signs at the screening visit |
| | | and within 72 hours prior to first dose |
| Physical measurements (including performance status) | X | Height and weight; Karnofsky performance status |
| Assessment of Signs and Symptoms | X | Within 14 days prior to randomization |
| Concomitant Medication collection | X | Within 14 days prior to randomization |
| Steroid Dose documentation | X | Within 14 days prior to randomization |
| Laboratory Tests | X | CBC w/differential; Chemistry panel including: |
| | | LDH, AST, ALT, ALP, T.Bili, BUN or serum urea |
| | | level, creatinine, Ca, Mg, Na, K, Cl, Glucose, |
| | | amylase, lipase, TSH, Free T4, Free T3, Hep B/C |
| | | (HBV sAG, HCV antibody), within 14 days prior to |
| | | randomization |
| Urinalysis | X | Dipstick; obtain within 14 days prior to randomization |
| Screening 12-lead ECG | X | Within 14 days prior to randomization |
| Pregnancy Test (WOCPB only) | X | |
| Cogstate Assessment | X | Practice Test 1 |

Tumor Assessment

| | | |
|---|---|---|
| Screening/Baseline Tumor | X | Within 21 days prior to randomization; Contrast- |
| Assessment | | enhanced MRI^{a}. The baseline MRI |
| | | assessment must be performed on a MRI qualified |
| | | to meet study specific acquisition parameters |
| | | (see Imaging Manual). |

^{a}When logistically feasible, the timing of the baseline MRI should be performed as close as possible to the randomization date but no longer than 21 days before. Additionally, MRI scans performed prior to this baseline may be collected by the sponsor.

TABLE 5
B: On-Study Assessments Nivolumab (N) and Bevacizumab (B) Arms (CA209143)

| | | |
|---|---|---|
| | Day 1 Week 1 and then every 2 weeks within | |
| Procedure | 3 days prior to dosing | Notes |

Safety Assessments

| | | |
|---|---|---|
| Targeted Physical Examination | X | Perform as clinically indicated |
| Vital Signs and Oxygen saturation | X | Pulse oximetry at rest and after exertion. |
| Physical Measurements | X | Weight and Karnofsky Performance Status (prior to |
| (including PS) | | each dose) |
| Adverse Events Assessment | Continuously | |
| Review of Concomitant | X | |
| Medications | | |
| Steroid medication | X | |
| documentation | | |
| Laboratory Tests | X | Obtain prior to each dose through Week 21 and then |
| | | obtain every 4 weeks thereafter; must be collected |
| | | within 72 horns prior to dose. Collect CBC |
| | | w/differential; Chemistry panel including: LDH, AST, |
| | | ALT, ALP, T.Bili, BUN or serum urea level, creatinine, |
| | | Ca, Mg, Na, K, Cl, Glucose, amylase, lipase, TSH with |
| | | reflexive Free T4, Free T3, |
| Urinalysis (For subjects in | X | Dipstick, within 72 hours prior to bevacizumab dose. |
| Arm B only) | | Subjects with 2+ or greater proteinurea dipstick reading |
| | | must undergo further assessment with a 24 hr urine |
| | | collection prior to being dosed. |
| Pregnancy Test (WOCBP only) | X (Day 1 Week 1 and then every 4 weeks) | Obtain at the specified schedule (i.e., every 4 weeks |
| | | [+/−1 week]) regardless of dose delays; |
| | | if collected with a dosing visit then obtain within |
| | | 24 hours prior to administration of study drug: |
| | | urine or serum |
| 12-leadECG | See note | As clinically indicated |

Pharmacokinetics/Immunogenicity Samples

| | |
|---|---|
| PK/Immunogenicity blood samples | X See note. |
| (nivolumab arm, only) | |

Exploratory Biomarker Samples

| | | |
|---|---|---|
| Serum (Soluble Biomarkers) | X See note | See Biomarker assessment |
| Peripheral Blood Mononuclear | X See note | See Biomarker assessment |
| Cells (PBMC) | | |
| Whole Blood for Gene Expression | X See note | See Biomarker assessment |
| and SNP (DNA) | | |
| Tumor sample at the time of | | If a biopsy or surgical resection is performed at the time |
| progression or suspected | | of progression or suspected progression, a tumor biopsy |
| progression | | sample (block or slides) should be submitted for central |
| | | neuropathologic review. (Note: Prior pre-study |
| | | treatment archival tumor specimens may be submitted |
| | | to the sponsor to aid in the histologic assessment when |
| | | available). |

Efficacy Assessments

| | | |
|---|---|---|
| Tumor assessments | X (Day 1 Week 7) | Contrast-enhanced MRI. Week 7 and 13, Day 1 and |
| | | then every 8 weeks (+/−1 week) |
| NANO Scale (Cohort 2 only) | X | Completed by study physician prior to dosing Day 1 |
| | | Week 1 and then with each MRI (but must be |
| | | completed before MRI scan results are reviewed with |
| | | the subject) |

Outcomes Research Assessments

| | | |
|---|---|---|
| EORTC QLQ-C30 and BN20 | X | Obtained prior to dosing Day 1 Week 1 and then with |
| | | each MRI prior to tumor assessment discussion |
| EQ-5D | X | Obtained prior to dosing Day 1 Week 1 and then with |
| | | each MRI prior to tumor assessment discussion |
| Health Care Resource Utilization | X | |
| Cogstate Assessment | X | Obtained Day 1 Week 1 (Practice test 2, Baseline) and |
| (Cohort 2 only) | | then Day 1 of Weeks 13, 21, 45, 69 and upon treatment |
| | | discontinuation (if >4 weeks from prior Cogstate |
| | | Assessment) |

Clinical Drug Supplies

| | | |
|---|---|---|
| IVRS Randomization | X | |
| Administer Study Treatment | X (within 3 working days of randomization) | Nivo is administered q 2 weeks; |
| | | Bevacizumab is administered q 2 weeks. |

C: On-Study Assessments Nivolumab+ Ipilimumab (N + I) Arm (CA209143; Cohort 1 and 1b, only)

| | | | |
|---|---|---|---|
| | Day 1 Week 1,4, 7, and | Day 1 Week 13 | |
| | 10 within 3 | and then every 2 | |
| Procedure | days prior to dosing | weeks | Notes |

Safety Assessments

| | | | |
|---|---|---|---|
| Targeted Physical Examination | X | X | Perform as clinically indicated |
| Vital Signs and Oxygen saturation | X | X | Pulse oximetry at rest and after exertion. |
| Physical Measurements | X | X | Weight and Karnofsky Performance Status |
| (including PS) | | | (prior to each dose) |
| Adverse Events Assessment | Continuously | → | |
| Review of Concomitant | X | X | |
| Medications | | | |
| Steroid Dose documentation | X | X | Within 72 hours prior to dose |
| Laboratory Tests | X | X (prior to dosing | Within 72 hours prior to dose. Collect CBC |
| | | on Day 1 Week 13 | w/differential; Chemistry panel including: |
| | | through Day 1 | LDH, AST, ALT, ALP, T.Bili, BUN or serum |
| | | Week 21 and then | urea level, creatinine, Ca, Mg, Na, K, |
| | | every 4 weeks | Cl, Glucose, amylase, lipase, TSH with |
| | | thereafter) | reflexive, Free T4, Free T3. |
| Pregnancy Test (WOCBP only) | X | X | Obtain at the specified schedule (i.e., |
| | | | every 4 weeks [+/−1 week]) |
| | | | regardless of dose delays; if collected with |
| | | | a dosing visit then obtain within 24 hours |
| | | | prior to administration of study drug: |
| | | | urine or serum; Obtain every 4 weeks |
| | | | (+/−1 week) regardless of dose delays |
| | | | during nivo monotherapy |
| 12-lead ECG | See note | See note | As clinically indicated |

Pharmacokinetics/Immunogenicity Samples

| | |
|---|---|
| PK/Immunogenicity blood samples | X See note. |

Exploratory Biomarker Samples

| | | | |
|---|---|---|---|
| Serum (Soluble Biomarkers) | X See note. | | See Biomarker assessment |
| Peripheral Blood Mononuclear | X See note. | | See Biomarker assessment |
| Cells (PBMC) | | | |
| Whole Blood for Gene Expression | X See note. | | See Biomarker assessment |
| and SNP (DNA) | | | |
| Tumor sample at the time of | See note | See note | If a biopsy or surgical resection is |
| progression or suspected | | | performed at the time of progression |
| progression | | | or suspected progression, a tumor biopsy |
| | | | sample (block or slides) should be |
| | | | submitted for analysis. |

Efficacy Assessments

| | | | |
|---|---|---|---|
| Tumor assessments | X (Day 1 Week 7 only) | X | Contrast-enhanced MRI at Day 1 Week 7 |
| | | | and 13 and then every 8 weeks |
| | | | (+/−1 week). |

Outcomes Research Assessments

| | | | |
|---|---|---|---|
| EORTC QLQ-C30 and BN20 | X | X (at the time of | Obtained prior to dosing Day 1 Week 1 |
| | | MRI) | and then with each MRI prior to |
| | | | tumor assessment discussion |
| EQ-5D | X | X (at the time of | Obtained prior to dosing Day 1 Week 1 |
| | | MRI) | and then with each MRI prior to |
| | | | tumor assessment discussion |
| Health Care Resource Utilization | X | | |

Clinical Drug Supplies

| | | | |
|---|---|---|---|
| IVRS Randomize | X | | |
| Administer Study Treatment | X (Nivo and Ipi on Day 1 Week 1, 4, | X | Nivo and Ipi combination are administered |
| | 7, and 10); Day 1 Week 1 dose within | | q 3 weeks x 4 doses; after the 4th ipi dose, |
| | 3 working days of randomization | | or when ipi is discontinued, nivo is |
| | | | administered every 2 weeks |

D: Follow-up Assessments (CA209143) - For all subjects

| | | | |
|---|---|---|---|
| | Follow up | | |
| | phase^{a} | Survival | |
| Procedure | Visits | Phase^{b} | Notes |

Safety Assessments

| | | |
|---|---|---|
| Targeted Physical Examination | X | |
| Adverse Event assessment | X | Beyond 100 days from the last dose of |
| | | study therapy, subjects will be followed |
| | | for ongoing drug-related adverse events |
| | | until resolved, return to baseline or |
| | | deemed irreversible, or until lost to |
| | | follow-up, withdrawal of study consent, |
| Laboratory Tests | X | CBC w/differential, LFTs, BUN, creatinine |
| | | and TSH for follow-up #1, repeat labs |
| | | at follow-up #2 if study drug related |
| | | toxicity persists. |
| Pregnancy test (for WOCBP) | X | |
| Review of Concomitant | X | |
| Medications | | |

Pharmacokinetics/Immunogenicity Samples

| | | |
|---|---|---|
| PK/Immunogenicity Blood Sample | X | Excluding subjects on bevacizumab. |

Outcomes Research Assessments

| | | | |
|---|---|---|---|
| EORTC QLQ-C30 and BN20 | X | | |
| EQ-5D | X | X | May be obtained through a telephone call |
| | | | or clinic visit |

Survival Status

| | | | |
|---|---|---|---|
| Survival Status | X | X | Every 3 months (clinic visit or telephone |
| | | | contact), during Survival phase, include |
| | | | subsequent anti-cancer therapy |

Efficacy Assessments

| | | | |
|---|---|---|---|
| Tumor Assessment | X | X | Only required for subjects who did not |
| | | | progress while on study therapy, including |
| | | | subjects who start subsequent anti- |
| | | | cancer therapy. Obtain at end of Week 6 |
| | | | and 12 (+/−1 week) and then every |
| | | | 8 weeks (+/−1 week thereafter. |
| | | | Tumor assessments will not be collected |
| | | | for subjects who are lost to |
| | | | follow-up or withdraw consent. |

^{a} Subjects must be followed for at least 100 days after last dose of study therapy. Follow-up visit #1 (FU1) occurs approximately 35 days (+/−7 days) after last dose or coinciding with the date of discontinuation (+/−7 days) if date of discontinuation is greater than 35 days after last dose. Follow up visit #2 (FU2) occurs approximately 80 days (+/−7 days) after FU1.
^{b} Survival visits = every 3 months from FU2

### NANO Assessment

Neurologic functioning is evaluated using the overall score of The Neurologic Assessment in Neuro-Oncology (NANO) which is administered by the investigator at the study.

The NANO is an objective, quick, user-friendly and quantifiable evaluation of nine major domains for subjects with brain tumors. The domains includes: gait, strength, ataxia, sensation, visual field, facial strength, language, level of consciousness, behavior and overall. Each domain is rated on a scale of 0 to 3 where 0 represents normal and 3 represents the worst severity. A given domain is scored non-evaluable if it cannot be accurately assessed due to preexisting conditions, co-morbid events and/or concurrent medications. The evaluation is based on direct observation/testing performed during routine office visits. The NANO scale is completed by the investigator or designated study physician prior to dosing Day 1 Week 1 (baseline) and then with each MRI (but completed prior to the review of the MRI scan results).

### Adverse Events

An Adverse Event (AE) is defined as any new untoward medical occurrence or worsening of a preexisting medical condition in a clinical investigation subject administered an investigational (medicinal) product and that does not necessarily have a causal relationship with this treatment. An AE can therefore be any unfavorable and unintended sign (such as an abnormal laboratory finding), symptom, or disease temporally associated with the use of investigational product, whether or not considered related to the investigational product.

The causal relationship to study drug is determined by a physician and should be used to assess all adverse events (AE). The casual relationship can be one of the following:
(a) Related: There is a reasonable causal relationship between study drug administration and the AE.
(b) Not related: There is not a reasonable causal relationship between study drug administration and the AE.

The term “reasonable causal relationship” means there is evidence to suggest a causal relationship. Adverse events can be spontaneously reported or elicited during open-ended questioning, examination, or evaluation of a subject. (In order to prevent reporting bias, subjects should not be questioned regarding the specific occurrence of one or more AEs.)

### Serious Adverse Events

A Serious Adverse Event (SAE) is any untoward medical occurrence that at any dose:
(a) results in death
(b) is life-threatening (defined as an event in which the subject was at risk of death at the time of the event; it does not refer to an event which hypothetically might have caused death if it were more severe)
(c) requires inpatient hospitalization or causes prolongation of existing hospitalization (see NOTE below)
(d) results in persistent or significant disability/incapacity
(e) is a congenital anomaly/birth defect
(f) is an important medical event (defined as a medical event(s) that may not be immediately life-threatening or result in death or hospitalization but, based upon appropriate medical and scientific judgment, may jeopardize the subject or may require intervention [e.g., medical, surgical] to prevent one of the other serious outcomes listed in the definition above.) Examples of such events include, but are not limited to, intensive treatment in an emergency room or at home for allergic bronchospasm; blood dyscrasias or convulsions that do not result in hospitalization.) Potential drug induced liver injury (DILI) is also considered an important medical event.

Suspected transmission of an infectious agent (e.g., pathogenic or nonpathogenic) via the study drug is an SAE. Although pregnancy, overdose, cancer, and potential drug induced liver injury (DILI) are not always serious by regulatory definition, these events must be handled as SAEs. Any component of a study endpoint that is considered related to study therapy (e.g., death is an endpoint, if death occurred due to anaphylaxis, anaphylaxis must be reported) should be reported as SAE.

The following hospitalizations are not considered SAEs in BMS clinical studies:
(a) a visit to the emergency room or other hospital department <24 hours, that does not result in admission (unless considered an important medical or life-threatening event)
(b) elective surgery, planned prior to signing consent
(c) admissions as per protocol for a planned medical/surgical procedure
(d) routine health assessment requiring admission for baseline/trending of health status (e.g., routine colonoscopy)
(e) medical/surgical admission other than to remedy ill health and planned prior to entry into the study. Appropriate documentation is required in these cases
(f) admission encountered for another life circumstance that carries no bearing on health status and requires no medical/surgical intervention (e.g., lack of housing, economic inadequacy, caregiver respite, family circumstances, administrative reason).
(g) Admission for administration of anti-cancer therapy in the absence of any other SAEs

### Biomarker Assessments

### Rationale and Aims for Biomarker Assessments

Previous studies with ipilimumab showed that increased expression of proliferation and polarization markers in CD4+ and CD8+ cells, decreased CCR7 and IL-7R on CD4+ T cells, and upregulated Granzyme B in CD8+ T cells may serve as pharmacodynamic indicators of the effects of ipilimumab. Increased activation markers ICOS and eomesodermin (EOMES) were significantly associated with fewer AEs and less favorable outcome, respectively, but baseline elevated expression of EOMES appears to predict favorable relapse-free survival in this analysis.

ICOS potentially could be a useful marker for CD4+ T-cell responses generated after CTLA-4 blockade. Human leukocyte antigen (HLA-DR) expression could also potentially serve as a T cell marker.

Previous studies have also shown that expression of immune-related genes such as CD8A, GZMA, LCK, CXCL-9, -10, -11, and CXCR3 in tumors at week 3 post-treatment was associated with the total lymphocyte infiltrate, clinical benefit, and overall survival. The preliminary results suggest that high pre-existing immune activity favors a clinical response to ipilimumab therapy.

Peripheral blood and tumor tissue will be collected prior to therapy. Peripheral blood samples will also be collected at selected time points on treatment. If a biopsy or surgical resection is performed at the time of progression or suspected progression, tumor sample (block or slides) should be submitted for analysis. If biomarker samples are drawn but study drug(s) is not administered, samples will be retained. A detailed description of each assay system is described below and a schedule of pharmacodynamic evaluations are measured. All biomarker sampling at time points when study drug is not administered are optional.

### Soluble Biomarkers

In this study serum samples were collected at baseline, during and after treatment from subjects enrolled on the trial to identify potential biomarkers with prognostic and predictive value for outcomes (response, progression-free survival and overall survival, toxicity). Preliminary results from this study will inform estimates for the phase 3 efficacy study. Soluble factors, such as cytokines, chemokines, soluble receptors, and antibodies to tumor antigens will be characterized and quantified by immunoassays in serum. Analyses may include, but not necessarily be limited to, soluble CD25, soluble PD-1, soluble LAG-3, and CXCL-9. Collected serum samples will also be used for the assessment of tumor antigen-specific responses elicited following treatment with monotherapy and combination therapy to explore which antitumor antibodies are most associated with clinical response. Antibody levels to cancer test antigens will be assessed by multiplex assays and enzyme-linked immunosorbent assay (ELISA).

### Immunophenotyping

The proportion of specific lymphocyte subsets and expression levels of T cell co-stimulatory markers in peripheral blood mononuclear cell (PBMC) preparations will be quantified by flow cytometry. Analyses may include, but not necessarily be limited to, the proportion of T, B, and NK cells, granulocytes, the proportion of memory and effector T cell subsets, and expression levels of PD-1, PD-L1, other B7 family members, ICOS, and Ki67.

### Ex Vivo Functional Assays

To explore whether monotherapy and/or combination therapy will restore T cell activation and function, peripheral blood mononuclear cells (PBMCs) will be isolated and cryopreserved. Assays of the functional status of effector T cells will be performed, including but not limited to, assays for interferon-gamma (IFN-γ) and CD107.

An exploration of the peripheral T cell compartment to GBM cancer stem cells will also be performed by one of several methods. These methods may include restimulation of peripheral blood using GBM cancer stem cells derived from immortalized cell lines followed by measurement of effector cytokines such as IFN-γ using ELISPOT. Alternatively, cancer stem cells purified from subject biopsies may be utilized in an ex vivo cytolytic assay using peripheral blood T cells.

### Peripheral Blood Gene Expression

The expression level of genes related to response to nivolumab monotherapy and nivolumab/ipilimumab combination therapy will be quantified molecular methods such as Affymetrix by microarray and/or quantitative reverse transcription polymerase chain reaction (RT-PCR) analysis in whole blood samples. Analysis may include, but not necessarily be limited to, genes associated with immune-related pathways, such as T cell activation and antigen processing and presentation.

### T Cell Repertoire Analysis

Low diversity of the peripheral T cell compartment has been shown to correlate with poor overall survival in metastatic breast cancer. A standing theory in immuno-oncology suggests a diverse and activated immune environment is better adept at eradicating tumor compared to a skewed repertoire of naïve and tolerized T cells. In order to explore whether a diverse T cell repertoire is predictive of response to therapy, next generation, high-throughput, DNA sequencing will be performed on DNA isolated from peripheral blood and tumor tissue to quantitate the composition of the T cell repertoire prior to, and during, monotherapy and combination therapy.

### Tumor Samples

### Tumor-Based Biomarker Measures

Tumor biopsy specimens will be obtained from consenting subjects prior to treatment with nivolumab monotherapy, nivolumab plus ipilimumab combination therapy, or bevacizumab to characterize immune cell populations and expression of selected tumor markers. Archival biopsy block or slides or fresh biopsy must be available for submission prior to randomization. If tumor tissue is not available, please contact the Medical Monitor to discuss possible inclusion of the subject in the protocol. Fresh biopsy collection prior to study therapy is preferred for subjects with accessible lesions but is not required. On-treatment biopsy samples are optional.

Fresh biopsies will be provided for biomarker analysis if accessible and deemed safe by the investigator and should be prioritized over submission of an archived sample. An archived biopsy prior to therapy is acceptable if the fresh biopsy cannot be obtained and if the archived tissue meets the defined criteria as stated below.

(a) An archived biopsy (block or slides) must contain tumor tissue;

(b) If an archived block is not available, 10 or more slides containing tumor are available for exploratory use

Whenever possible the archival tumor sample should be obtained from a time point after the subject has completed other systemic therapy (including treatment with temozolomide) and prior to randomization. Biopsy samples may be used for the following assessments:

### Characterization of Tumor Infiltrating Lymphocytes (TILs) and Tumor Antigens

Immunohistochemistry (IHC) will be used to assess the number and composition of immune infiltrates in order to define the immune cell subsets present within formalin-fixed, paraffin embedded (FFPE) tumor tissue before and after exposure to therapy. These IHC analyses will include, but not necessarily be limited to, the following markers: CD4, CD8, FOXp3, PD-1, PD-L1, and PD-L2.

Cancer stem cell phenotyping via IHC will also be performed to assess the phenotype, location, and density of cancer stem cells in the biopsy.

In cases where fresh tumor is available prior to therapy and/or on treatment, separation and isolation of TIL from tumor and stromal cells may be performed. Isolated TIL may be utilized to assess the gene expression, phenotype, and function of immune cells. Techniques such as next generation sequencing of TIL and flow-cytometry to characterize the phenotype, function, and specificity may be performed.

### Characterization of T Cell Repertoire

As described above, DNA sequencing will be performed on pre- and posttreatment tumor tissue to assess the composition of the T cell repertoire. DNA will be isolated from either the FFPE tumor block or from RNAlater or equivalent preparations.

### Gene Expression Profiling

Tumor biopsies that are collected in RNAlater or equivalent fixative will be examined for mRNA gene expression by Affymetrix gene array technology and/or quantitative real-time polymerase chain reaction (qPCR) to detect expression of selected immune related genes.

### Tumor Genotyping, Mutational Analysis, and Tumor Antigen Profiling

RNA and DNA from tumor samples will be analyzed using whole-exome and transcriptome sequencing to determine the number of mutations found within a given sample relative to a normal host tissue, such as adjacent non-transformed cells or PBMC. Mutations that are detected will be analyzed for their ability to bind the MHC I and MHC II proteins using prediction algorithms, such as NetMHC. Evaluating the ability of tumor mutations to bind MHC molecules will provide evidence that these mutations are serving as antigens that are recognized the immune system and are potential rejection antigens.

### Tumor Sample Collection Details

### Fresh Biopsy:

Fresh biopsies at baseline should be prioritized over archived samples and are strongly encouraged on-treatment.

Tumor samples obtained from bone metastases are not considered acceptable for PD-L1 testing because the PD-L1 assay does not include a decalcification step. For any cases where the only tumor tissue available is from a bone metastasis lesion, please discuss further with the study Medical Monitor.

Biopsy samples should be excisional, incisional or core needle using a gauge needle that is utilized within the institution. In general, a 16 or 18 gauge needle is used for core biopsies.

It is recommended that samples be fixed in 10% Neutral-buffered formalin for 24-48 hours. Tumor tissue samples should not be shipped in formalin as the temperature and length of fixation cannot be controlled during shipping.

If slides are submitted, the recommended tissue section thickness is 4 microns and the slides must be positively charged. Slides should be shipped refrigerated at 2-8° C.

Sample shipments should include a completed requisition form containing collection date, collection method, primary/met, site, fixation conditions, and a copy of Pathology report, if available.

If a fresh biopsy is taken, up to 4 core biopsies are recommended. An assessment of biopsy quality by a pathologist is strongly encouraged at the time of the procedure. The tumor tissue that is obtained from these biopsies will be divided three ways in the following priority order: into formalin for fixation and paraffin-embedding, in RNALater for RNA/DNA extraction and prepared for tumor infiltrating lymphocyte (TIL) isolation.

The investigator, in consultation with the radiology staff, must determine the degree of risk associated with the procedure and find it acceptable. Biopsies may be done with local anesthesia or conscious sedation. Institutional guidelines for the safe performance of biopsies should be followed. Excisional biopsies may be performed to obtain tumor biopsy samples. Invasive procedures that require general anesthesia should not be performed to obtain a biopsy specimen. However, if a surgical procedure is performed for a clinical indication, excess tumor tissue may be used for research purposes with the consent of the subject.

### Archived Biopsy:

Minimum of 1 formalin-fixed paraffin embedded (FFPE) tumor tissue block (preferred) OR minimum of 10 FFPE unstained sections are required for assessment of PD-L1 status and other biomarker evaluations.

TABLE 6
Biomarker Sampling Schedule

| | Collection Timing | | | | | |
|---|---|---|---|---|---|---|
| | | PBMC^{e} | | | | |
| | Serum | | Ex vivo | Tumor | Whole Blood | |
| | Soluble | Immuno- | Functional | Tumor | Gene | |
| Study Day | Biomarkers | phenotyping | Assay | Biopsy | Expression | SNP |
| Screening | | | | X^{b} | | |
| Day 1 | X | X | X | | X | X |
| (D 1/Wk 1) | | | | | | |
| Day 8 | X | X | | | | |
| D 1/Wk 2) | | | | | | |
| Day 15 | X | X | | | | |
| (D 1 Wk 3) | | | | | | |
| Day 29 | X | X | | | | |
| (D 1 Wk 5) | | | | | | |
| Day 43 | X | X | X | X^{c} | X | |
| (D 1 Wk 7) | | | | | | |
| Day 85 | X | X | | | | |
| (D 1 Wk 13) | | | | | | |
| Day 99 | X | X | X | | X | |
| (D 1 Wk 15) | | | | | | |
| Upon | X | X | X | X^{c} | X | |
| Progression^{d} | | | | | | |

^{a} Biomarker sampling occurs prior to dosing of study drug and can occur +/−2-5 days from the scheduled time.
^{b}Tumor biopsy prior to therapy is mandatory. If a recent (as defined in protocol) archived biopsy is not available at screening, a fresh biopsy will be taken at any point prior to treatment. Subjects for whom tumor biopsy is not available at the time of enrollment must be discussed with the medical monitor regarding possible inclusion.
^{c}Optional biopsies on-treatment and upon progression and may be taken at the discretion of the investigator
^{d}Samples from subjects that have confirmed progression are optional.
^{e}Biomarker samples on non-study drug dosing days for the monotherapy or combination treatment are optional.

Detailed instructions of the obtaining, processing, labeling, handling, storage and shipment of specimens will be provided in a separate Procedure Manual at the time of study initiation.

Demographics and baseline characteristics of the patients enrolled in the study are seen in Table 7.

TABLE 7
Demographics and Baseline Characteristics

| | Nivolumab | Nivolumab 1 mg/kg + |
|---|---|---|
| | 3 mg/kg | ipilimumab 3 mg/kg |
| Characteristic | N = 10 | N = 10 |
| Median age (range), | 59 (42-73) | 57 (37-68) |
| years | | |
| Age, years, n (%) | | |
| <65 | 6 (60) | 7 (70) |
| >65-<75 | 4 (40) | 3 (30) |
| Gender, n (%) | | |
| Male | 5 (50) | 6 (60) |
| Female | 5 (50) | 4 (40) |
| Race, n (%) | | |
| White | 8 (80) | 10 (100) |
| Black/African-American | 2 (20) | 0 |
| Kamofsky performance score, n (%) | | |
| 90 | 7 (70) | 6 (60) |
| 80 | 1 (10) | 1 (10) |
| 70 | 2 (20) | 3 (30) |
| Histopathologic diagnosis, n (%) | | |
| Glioblastoma | 9 (90) | 10 (100) |
| Gliosarcoma | 1 (10) | 0 |
| MGMT gene promoter methylation | | |
| status, n (%) | | |
| Methylated | 2 (20) | 2 (20) |
| Unmethylated | 4 (40) | 6 (60) |
| Missing | 4 (40) | 2 (20) |

### Example 2

### Clinical Review of Cohort 1 Data

The proposed dosing regimen for nivolumab and ipilimumab in Cohort 1 and 1b was based upon safety and tolerability data from the use of nivolumab and ipilimumab in other tumor types. Although the proposed dosing regimen was expected to be tolerable in subjects with GBM, this study initially included two safety lead-in cohorts (Cohort 1 and 1b) prior to initiation of the efficacy study comparing nivolumab monotherapy versus bevacizumab (Cohort 2). Approximately 10 subjects with recurrent GBM were randomized to each of the two investigational treatment groups in Cohort 1 (Arm N and Arm N+I). Up to 20 subjects with recurrent GBM will be treated in Cohort 1b (Arm N+I_1b).

Tolerability and safety of a treatment arm was determined after all subjects per arm have completed four doses or discontinued dosing prior to completing four doses. Tolerability beyond four doses may also be taken into consideration. The tolerability criteria used to advance to Cohort 2 was based on drug related adverse events leading to permanent discontinuation in the safety lead-in cohorts and include the following:
If no more than one-third of the subjects in a given treatment arm permanently discontinue study medication prior to completing four doses due to treatment related adverse events then this dose cohort will be deemed as tolerable and enrollment in Cohort 2 may proceed;
If more than one-third of subjects in a treatment arm permanently discontinue study medication prior to completing four doses due to treatment-related adverse events, then the safety and tolerability of that treatment arm will be reviewed with the DMC prior to randomizing any additional subjects. A decision will be made by the sponsor whether to continue enrollment or advance either treatment arm into Cohort 2.

Treatment with nivolumab monotherapy in Cohort 1 met the protocol prespecified safety and tolerability profile detailed above to advance to a randomized study versus bevacizumab in Cohort 2.

Patient disposition is seen in Table 8. No patients receiving nivolumab monotherapy discontinued treatment due to drug toxicity. There were no deaths due to drug toxicity in the monotherapy or combination treatment arms. At the time of cutoff (Sep. 9, 2015), one patient in the nivolumab monotherapy arm continues to receive nivolumab after 17.5 months of treatment.

TABLE 8
Patient Disposition

| | Nivolumab | Nivolumab 1 mg/kg + |
|---|---|---|
| | 3 mg/kg | ipilimumab 3 mg/kg |
| | N = 10 | N = 10 |
| Patients in the treatment period, n (%) | | |
| Continuing on treatment | 1 (10) | 0 |
| Not continuing on treatment | 9 (90) | 10 (100) |
| Reason for not continuing on treatment, n (%) | | |
| Disease Progression | 9 (90) | 6 (60) |
| Study Drug Toxicity | 0 | 3 (30) |
| Death | 0 | 1 (10) |
| Patients in the study, n (%) | | |
| Continuing in the study | 3 (30) | 1 (10) |
| Died¹ | 7 (70) | 9 (90) |

The investigator-assessed objective responses per RANO criteria are see in Table 9. The majority of patients (6/10) treated with nivolumab monotherapy experienced stable disease or better. Four patients in the combination arm achieved best responses of stable disease. An exploratory analysis of overall survival is seen in Table 10. NE: not estimated. ¹ Patient deaths in both treatment arms were due to disease.

TABLE 9
Investigator-assessed objective responses per RANO criteria

| | Nivolumab | Nivolumab 1 mg/kg + |
|---|---|---|
| | 3 mg/kg | ipilimumab 3 mg/kg |
| Best overall response, n (%)² | N = 10 | N = 10 |
| Complete Response | 0 | 0 |
| Partial Response | 1 (10) | 0 |
| Stable Disease | 5 (50) | 4 (40) |
| Progressive Disease | 3 (30) | 6 (60) |
| Unable to Determine | 1 (10) | 0 |

TABLE 10
Exploratory Analysis of Overall Survival

| | Nivolumab | Nivolumab 1 mg/kg + |
|---|---|---|
| | 3 mg/kg | ipilimumab 3 mg/kg |
| | N = 10 | N = 10 |
| Median, months | 10.5 (4.1-NE) | 9.3 (4.0-12.7) |
| (95% CI) | | |
| Range, months | 4.1-17.6 | 4.5-16.0 |
| 6-month rate | 70.0% (32.9-89.2) | 80.0% (40.9-94.6) |
| (95% CI) | | |
| 9-month rate | 60.0% (25.3-82.7) | 60.0% (25.3-82.7) |
| (95% CI) | | |
| 12-month rate | 40.0% (12.3-67.0) | 30% (7.1-57.8) |
| (95% CI) | | |

### Example 3

### Cohort 1: Clinical Case Example of Pseudoprogression

A 67-year old white female subject was initially diagnosed with right occipital GBM in April 2013. The subject underwent a right temporal resection in April 2013 followed by concurrent chemoradiotherapy from May to July of 2013 and everolimus and temozolomide treatment from May to August of 2013. The subject experienced a first recurrence of the GBM in March 2014. This subject was randomized to the nivolumab 3 mg/kg treatment arm, beginning treatment in April 2014. The subject received ten doses of nivolumab 3 mg/kg

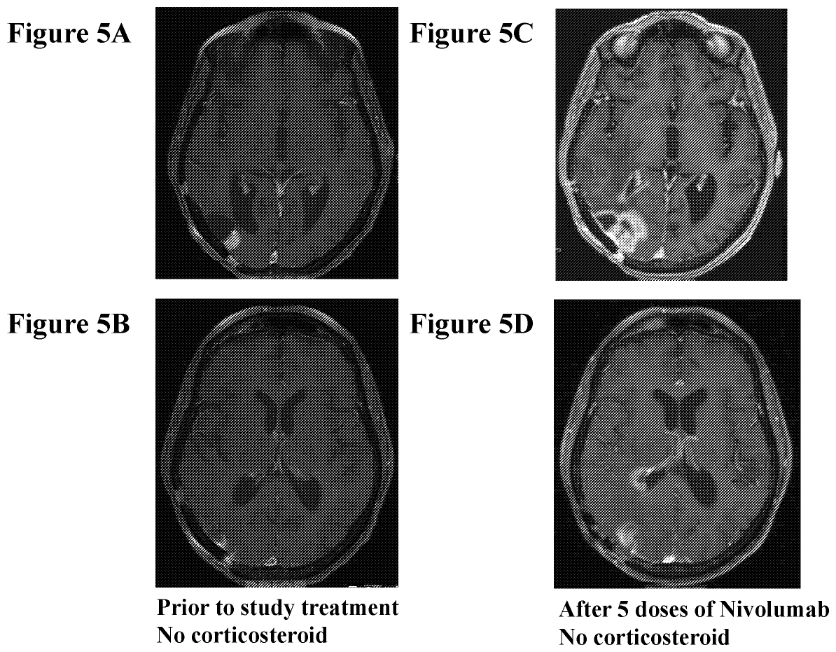

Suspected progression of the GBM was observed by MRI. At baseline, the GBM lesion measured 12 mm×10 mm. Following treatment, at day 43, the lesion measured 20 mm×17 mm; and at day 73, the lesion measured 20 mm×40 mm. MRI images of the subject before treatment (FIGS. 5A and 5B) and after five doses of nivolumab are shown in FIGS. 5C and 5D. The MRI scan after five doses of nivolumab (3 mg/kg Q2W) showed increased contrast enhancement of the temporal lobe lesion and new periventricular contrast enhancement; decreased vision was reported. MRIs on days 43 and 73 suggested evidence of progression. The histopathology of the resected tumor revealed immune infiltration consistent with treatment-related changes.

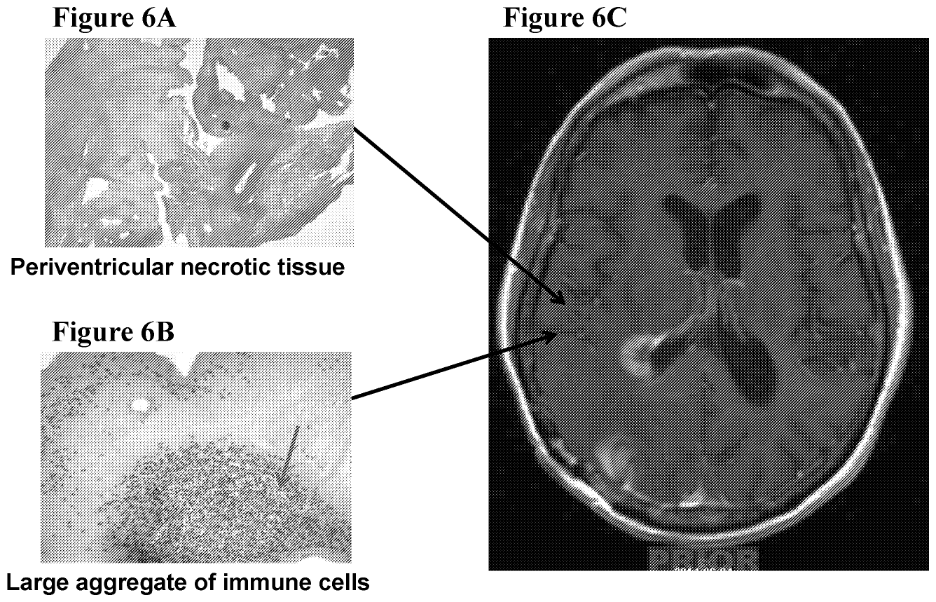

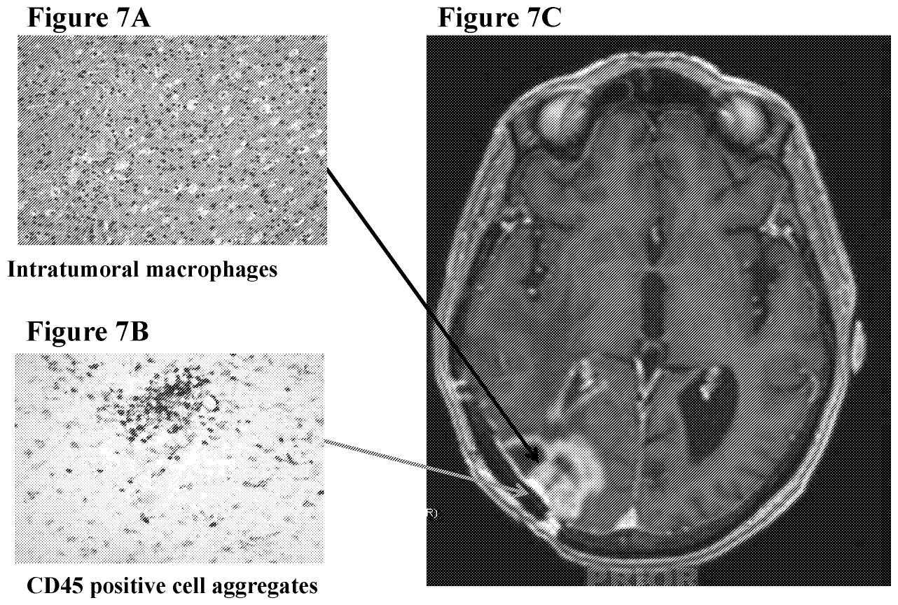

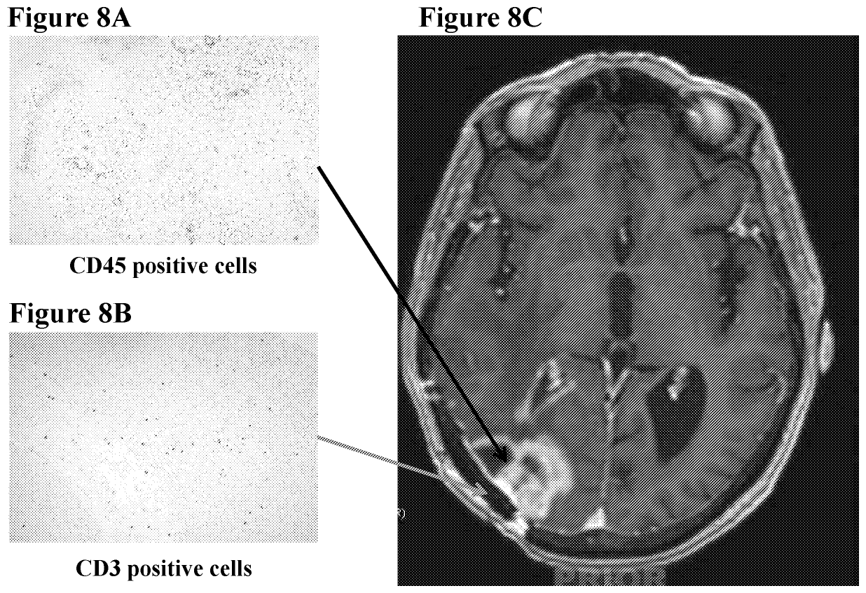

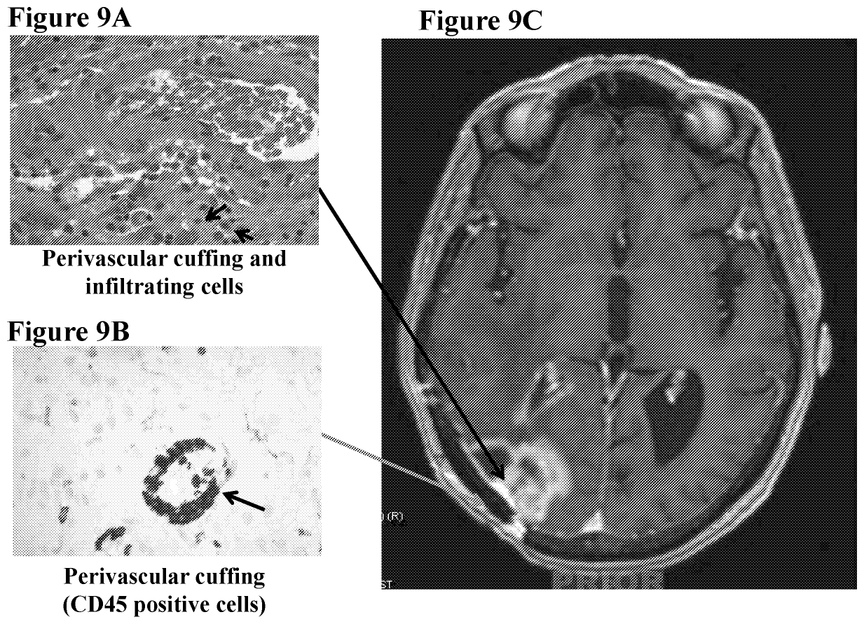

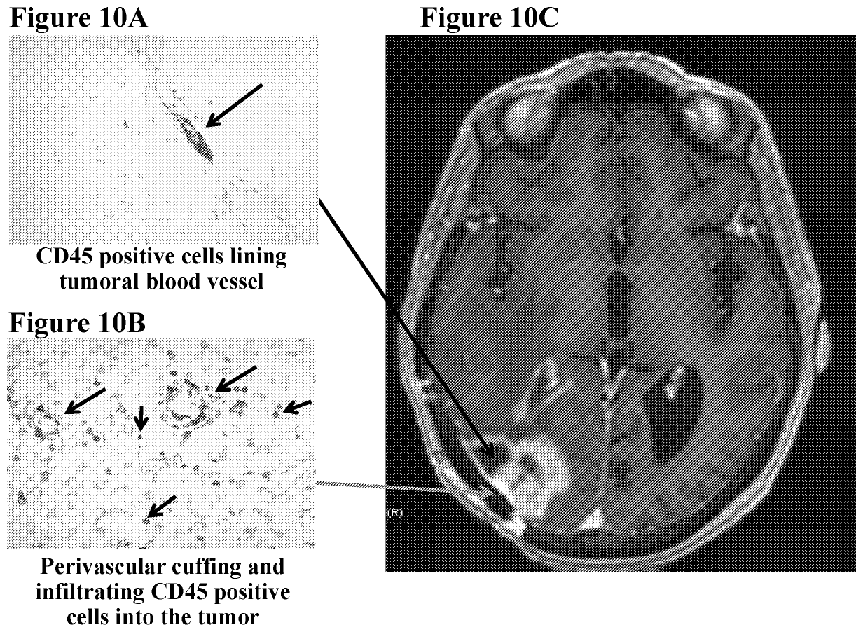

The GBM was resected to determine whether the tumor had progressed or pseudo-progressed. Histological analysis of the resected revealed periventricular necrotic tissue and large aggregation of immune cells (FIGS. 6A-C); intratumoral macrophages and CD45 positive cell aggregates (FIGS. 7A-C); CD45 positive cells and CD3 positive cells (FIGS. 8A-C); and perivascular cuffing and infiltrating cells (FIGS. 9A-C) within the resected tumor tissue. In addition, CD45 positive cells were observed lining tumor blood vessels as shown in FIGS. 10A-C. This neuropathology is consistent with significant treatment-related changes, and the subject resumed treatment with nivolumab 3 mg/kg.

The patient received treatment for a total of 227 days. The patient died 397 days after randomization. The patient had distant disease progression in the hippocampus and right hypothalamus (histologically proven on autopsy) 4 months after resuming nivolumab following surgery.

### Example 4

### Cohort 1: Clinical Case Example of Partial Response

A 42-year old white male subject was initially diagnosed with left temporal GBM in October 2013. The subject underwent surgical resection in October 2013, and the resected tissue was found to be MGMT methylated and EGFRvIII positive. The subject underwent standard focal brain chemotherapy from November to December 2013, followed by two cycles of adjuvant temozolomide treatment in February 2014. Progressive worsening was observed by MRI in February and early and late March 2014, with increasing relative cerebral blood volume (rCBV). The subject experienced a first recurrence of the GBM on Mar. 31, 2014. The subject was then treated with lamotrigine and alprazolam. This subject was randomized to the nivolumab 3 mg/kg treatment arm.

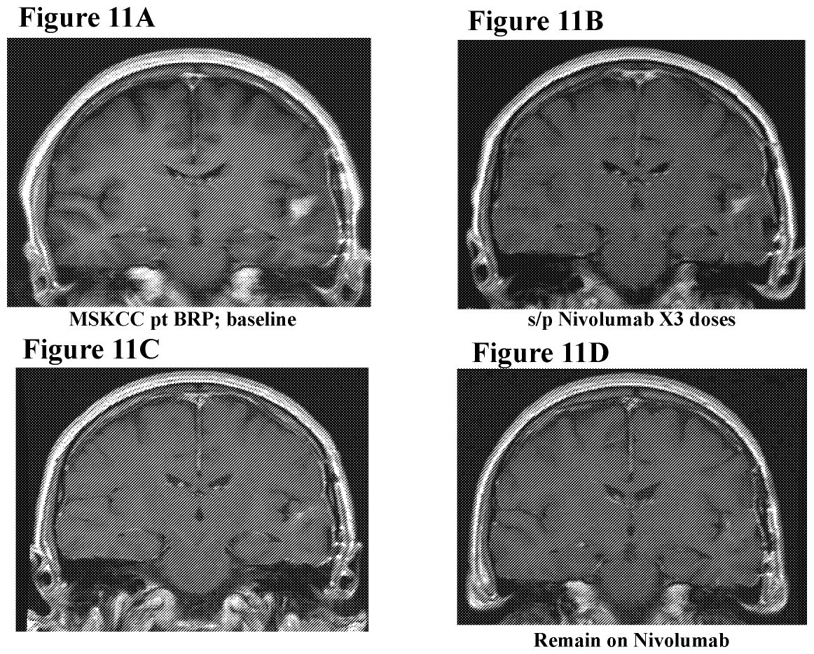

The subject displayed partial response according to RANO criteria. At baseline, the GBM lesion measured 27 mm×10 mm. Following treatment, at day 44, the GBM lesion measured 26 mm×9 mm; and at day 85, the GBM lesion measured 23 mm×4 mm. The subject's Karnosky performance status was 90 at baseline and at all subsequent visits. MRI images of the subject before treatment, after three doses of nivolumab, and at two later time points following continued treatment with nivolumab are shown in FIG. 11.

### Example 5

Cohort 1 Arm N+I: Clinical Case Examples Following Treatment with Nivolumab (1 mg/kg)+Ipilimumab (3 mg/kg)

A 45-year old female subject received a first dose of nivolumab (1 mg/kg)+ipilimumab (3 mg/kg) on Feb. 12, 2014. The subject completed three cycles of treatment, with the final dose administered on Apr. 2, 2014. Treatment was then discontinued due to severe diabetic ketoacidosis. Additional AEs on treatment, at different time points, included: grade 2 pneumonitis, grade 4 elevation of lipase, grade 3 elevation of amylase, and grade 1 elevations of AST/ALT. The subject's brain MRI on Sep. 10, 2014 showed disease progression. The subject was clinically worse as well. The subject was then started on bevacizumab monotherapy and is now doing well clinically. Compassionate access of nivolumab monotherapy has been approved, and treatment is forthcoming.

A 57-year old female subject received a first dose of nivolumab (1 mg/kg)+ipilimumab (3 mg/kg) on Apr. 17, 2014. The subject developed severe, grade 3 delirium on May 15, 2014 (C2 D8) requiring admission. All infectious and metabolic work up was negative, and an EEG only showed slow waves. The subject recovered to base line following two days of high-dose steroid treatment. The subject was then switched to nivolumab monotherapy on Jun. 19, 2014. Subsequently, the subject received three doses of nivolumab 3 mh/kg, with the last dose administered on Jul. 17, 2014. On Jul. 31, 2014, before administration of a sixth dose of nivolumab, the subject reported grade 2 dyspnea, fatigue, and weakness in her left side. In addition, the subject was on a tapering dose of prednisone for diarrhea (20 mg/day). The subject's brain MRI showed interval increase in enhancement and FLAIR signal. These changes were not measurable. Treatment was discontinued per the subject's request. A repeat brain MRI on Aug. 19, 2014 showed clinical deterioration with left sided hemiparesis and increased enhancement. The subject underwent surgical resection Aug. 27, 2014. Analysis of the resected tumor tissue suggested tumor progression. The subject came off trial and is currently being treated with bevacizumab alone. The subject declined addition of CCNU. The subject's other toxicities on this study included: grade 3 asymptomatic elevation of lipase, grade 2 diarrhea, grade 2 asymptomatic elevation of ALT, and grade 1 elevation of AST.

### Example 6

Cohort 1 Arm N: Clinical Case Example Following Treatment with Nivolumab (3 mg/kg) Monotherapy

A 67-year old female subject received a first dose of nivolumab 3 mg/kg monotherapy on Mar. 24, 2014. The subject completed ten doses of nivolumab, with the last dose administered on Nov. 5, 2014. The subject's brain MRI on May 5, 2014, following three treatments, showed disease progression as per RANO, particularly around the surgical cavity and new periventricular area. The subject agreed with treatment beyond progression and received two subsequent infusions, with the fourth treatment on May 7, 2014 and the fifth treatment on May 21, 2014. On Jun. 4, 2014, prior to a sixth infusion, the subject presented with visual field cut. A brain MRI showed further growth of the primary tumor at the occipital area and an abnormal finding in the periventricular area. Treatment was not administered at that time, and the subject had surgical resection on Jun. 12, 2014. Pathology review showed viable tumor in samples from the occipital area with infiltrating T-lymphocytes. There was significant necrosis (radiation necrosis) in the same area. The periventricular area showed only necrosis and fibrinous exudate (radiation necrosis) and large aggregates of lymphocytes with no tumor.

The subject recovered well from surgery. A brain MRI on Jul. 10, 2014, four weeks after resection, did not show tumor growth. The subject then re-started nivolumab monotherapy, receiving a sixth dose on Jul. 15, 2014 and a seventh dose on Jul. 30, 2014. Approximately one week after re-starting nivolumab monotherapy, the subject reported worsening neurologic symptoms (grade 2) including worsening of visual field cut. The subject was not on a steroid treatment at that time. A brain MRI showed interval increase in FLAIR signals and increase in diffuse rim enhancement in the postoperative bed. There was persistent restricted diffusion consistent with treatment change. As per protocol, treatment was delayed. The subject was started on dexamethasone 4 mg BID, and the subject's clinical condition improved. A brain MRI on Aug. 28, 2014 revealed improvement with resolution of rim enhancement noted in previous MRI. The subject re-started nivolumab monotherapy on Sep. 25, 2014, and dexamethasone treatment has been gradually tapered down to 3 mg/day with no significant neurologic change. The subject's condition will continued to be monitored by MRI as per protocol.

### Example 7

Cohort 1b Arm N+I(1b): Clinical Case Examples Following Treatment with Nivolumab (3 mg/kg)+Ipilimumab (1 mg/kg)

A 46-year old male subject received a first dose of nivolumab (3 mg/kg)+ipilimumab (1 mg/kg) on Jun. 27, 2014, a second dose on Jul. 18, 2014, a third dose on Aug. 11, 2014, and a fourth dose on Sep. 4, 2014. The subject further completed four cycles of nivolumab monotherapy, with the final dose administered on Nov. 4, 2014. The subject tolerated treatment very well with no side effects. The subject experienced a significant clinical improvement in movement and cognition. The subject's brain MRI showed a decrease in tumor size (stable as per RANO) despite starting as a very large tumor. The subject is on dexamethasone 4 alternating with 2 mg daily. The subject's condition will continued to be monitored by MRI as per protocol.

A 74-year old male subject received a first dose of nivolumab (3 mg/kg)+ipilimumab (1 mg/kg) on Jul. 9, 2014, a second dose on Jul. 31, 2014, a third dose on Aug. 21, 2014, and a fourth dose on Sep. 11, 2014. The subject further completed three cycles of nivolumab monotherapy, with the final dose administered on Oct. 30, 2014. Adverse events on treatment, at different time points, included: grade 2 rash and diarrhea, grade 2 pneumonitis, grade 1 elevation of lipase and ALT, and mild neuropathy. These AEs were improved with high-dose steroid treatment. The subject also received methylprednisolone 32 mg/day. The subject's brain MRI on Sep. 29, 2014, following four cycles of combination therapy and prior to the first cycle of nivolumab monotherapy, showed disease progression (as per RANO). The subject continued treatment, and a subsequent brain MRI was stable. The subject's condition will continue to be monitored by MRI as per protocol.

A 62-year old male subject received a first dose of nivolumab (3 mg/kg)+ipilimumab (1 mg/kg) on Jul. 28, 2014, a second dose on Aug. 25, 2014, a third dose on Sep. 8, 2014, and a fourth dose on Oct. 21, 2014. Adverse events on treatment included: grade 3 rash and grade 2 fatigue, which were both resolved after steroid treatment. The subject's brain MRI on Sep. 8, 2014, prior to the third cycle, showed disease progression as per RANO. The subject elected to continue treatment beyond progression. However, the subject took some time to decide. Repeat brain MRI at the 8-week time point showed stable disease from last MRI. However, the results were still considered progression from baseline, and the subject elected to have surgical resection of the tumor on Nov. 12, 2014. The subject will decide whether to continue treatment based on the results of the resection.

### Example 8

T-cell maturation, activation, and function in patients with GBM treated with nivolumab±ipilimumab was evaluated. And the frequency and function of Tregs and myeloid-derived suppressor cells in patients with GBM treated with nivolumab ipilimumab was determined.

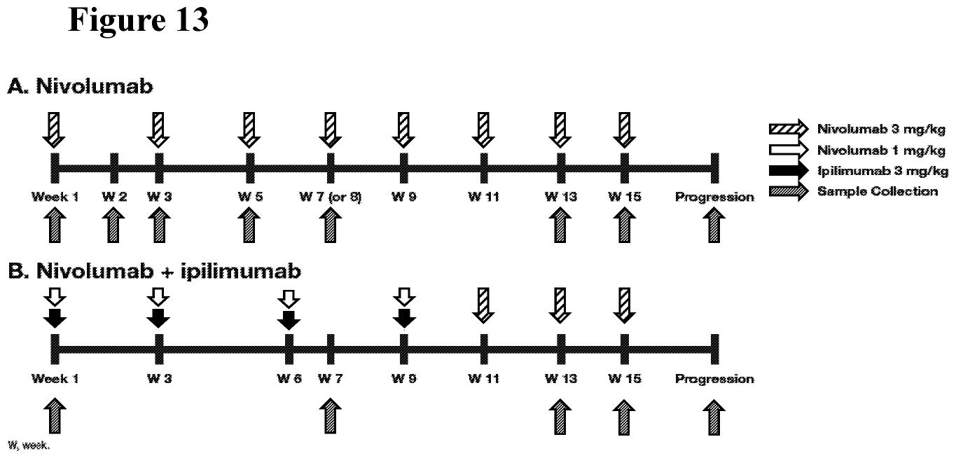

Analyses were performed on peripheral blood drawn from patients at predetermined time points (FIG. 13) and tumor tissue samples were obtained if resection was performed at the time of progression or suspected progression. Baseline and on-treatment samples were obtained from patients (n=20) randomized 1:1 to receive either nivolumab 3 mg/kg every 2 weeks (Q2W) or nivolumab 1 mg/kg+ipilimumab 3 mg/kg every 3 weeks followed by nivolumab 3 mg/kg Q2W (FIG. 13). Phenotypic, maturation, and proliferation analyses were performed using flow cytometry with population-specific antibody panels (Table 11). Cytokine release analyses were carried out on supernatants from sorted Tregs (CD4+CD25+CD127−)±stimulation (PMA+ionomycin). The anti-tumor-associated antigen (TAA) activity of peripheral blood mononuclear cells (PBMCs) and tumor-infiltrating lymphocytes (TILs) was evaluated. Cells were stimulated with interleukin (IL)-2, IL-15, and TAA peptides; after 9 days of culture with IL-2 and IL-15, cells were restimulated with IL-2, IL-15, and TAA peptides. Production of interferon (IFN)γ/granzyme B was assayed using a dual-color FluoroSpot assay.

TABLE 11
Antibody Panels for Immune Profiling

| | Marker | Stain | Purpose | Fluorophore |
|---|---|---|---|---|
| Tumor Reactive T cells | Zombie | Surface | Dead cell exclusion | Near IR |
| (14-color) | CD3 | Intracellular | T cells | BUV 395 |
| | CD4 | Intracellular | CD4+ (helper) T | BUV805 |
| | | | cells | |
| | CD8 | Intracellular | CD8+ (cytotoxic) T | BV510 |
| | | | cells | |
| | CD28 | Surface | co-stimulation | PerCP-Cy5.5 |
| | CD223 (LAG-3) | Surface | Activation/exhaustion | PE-Cy7 |
| | CD38 | Surface | Activation | APC-R700 |
| | CD197(CCR7) | Surface | Maturation | PE-Dazzle594 |
| | CD45RA | Surface | Maturation | APC |
| | CD366 (TIM-3) | Surface | Activation/exhaustion | BV421 |
| | CD279 (PD-1) | Surface | Activation/exhaustion | PE |
| | CD152 (CTLA-4) | Intracellular | Regulation | BV786 |
| | Ki67 | Intracellular | Proliferation | Alexa 488 |
| | CD278 (ICOS) | Surface | Activation | BV650 |
| Treg and MDSC (12-color, | Zombie | Surface | Dead cell exclusion | Near IR |
| 15-marker) | CD3 | Intracellular | T cells | BUV 395 |
| | CD4 | Intracellular | CD4+ (helper) T | BUV805 |
| | | | cells | |
| | CD8 | Intracellular | CD8+ (cytotoxic) T | BV510 |
| | | | cells | |
| | CD16 | Surface | Lineage exclusion | PE-Cy7 |
| | CD19 | | channel | |
| | CD20 | | | |
| | CD56 | | | |
| | CD25 | Surface | Tregs | APC-R700 |
| | CD 127 | Surface | Tregs | PE-Dazzle594 |
| | Ki67 | Intracellular | Proliferation | Alexa 488 |
| | CD152 (CTLA-4) | Intracellular | Regulation | BV786 |
| | FoxP3 | Intracellular | Tregs | PE |
| | CD14 | Surface | MDSCs | PerCP-Cy5.5 |
| | HLR-DR | Surface | MDSCs | BV605 |

ICOS, inducible costimulation; LAG-3, lymphocyte activation gene 3; MDSCs, myeloid-derived suppressor cells; ITM-3, T-cell immunoglobulin and mucin domain 3

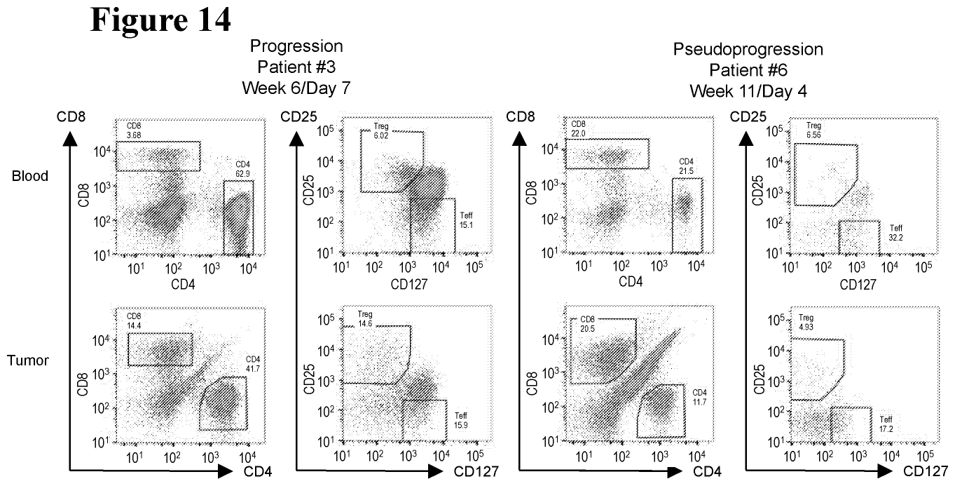

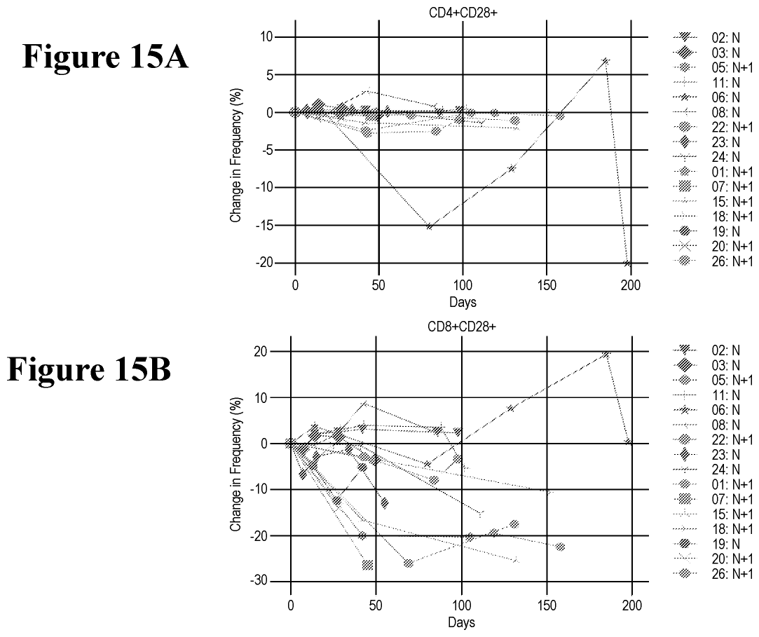

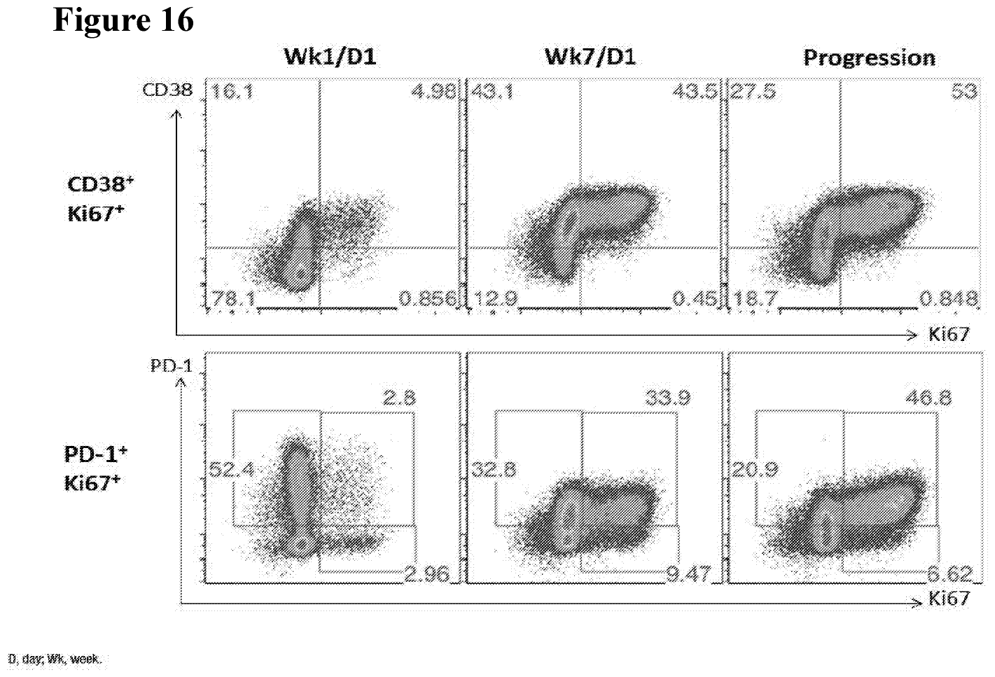

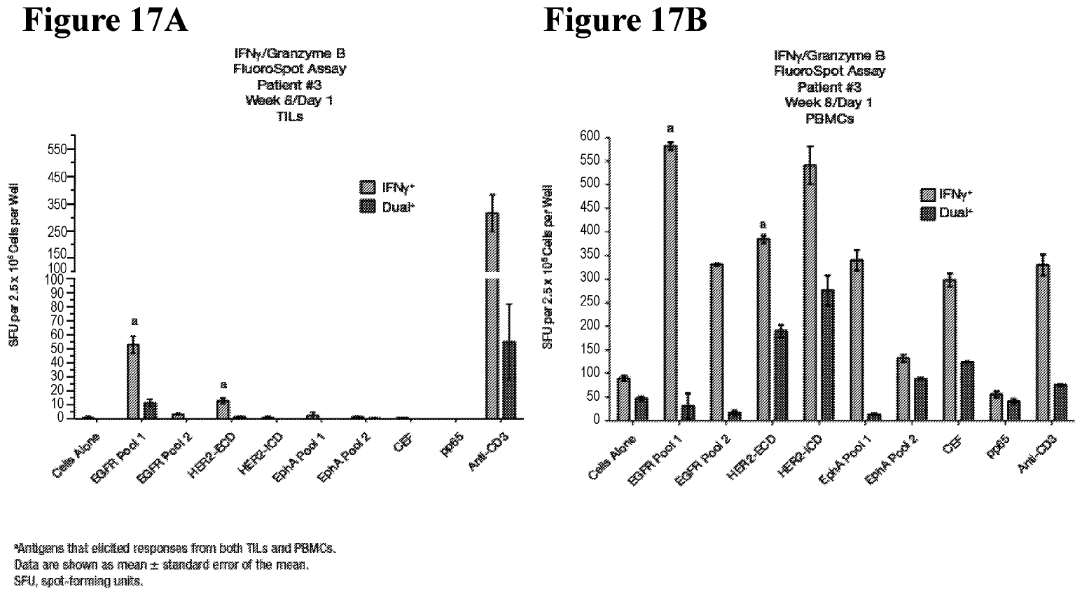

Flow cytometric analysis identified clean populations of CD4 T cells, CD8 T cells, and Tregs in both peripheral blood and tumor specimens (FIG. 14). Unique immune profiles were observed in the immune infiltrates from patients who experienced progression v. pseudoprogression, including striking qualitative differences in CD8:Cd4 ratios (FIG. 14). Patients treated with nivolumab+ipilimumab experienced a decrease in the relative frequency of CDD+CD28+ T lymphocytes compared to patients treated with nivolumab monotherapy (FIG. 15). Treatment with nivolumab+ipilimumab increased proliferation and activation of CD8+CD28− T cells over baseline (FIG. 16). Upon restimulation, TILS and PBMCs isolated from patients treated with nivolumab monotherapy showed increased anti-TAA activity (FIG. 17).

Nivolumab treatment increase production of IFNγ by TREGS, suggesting a reduction in immunosuppressive activity. Treatment with nivolumab resulted in functional responses to TAAs in both TILs and PBMCs. The immune profile of tumor-infiltrating T cells was highly distinctive in one patient experiencing immune-related treatment effects in tumor on nivolumab treatment.

### Example 9

Temozolomide in Combination with Ipilimumab and/or Nivolumab in Treating Patients with Newly Diagnosed GBM or Gliosarcoma

This randomized phase I trial studies the safety and best of temozolomide when given together with ipilimumab, nivolumab, or a combination of both in treating patients with newly diagnosed GBM or gliosarcoma. Drugs used in chemotherapy, such as temozolomide, work in different ways to stop the growth of tumor cells, either by killing the cells, by stopping them from dividing, or by stopping them from spreading. Monoclonal antibodies, such as ipilimumab and nivolumab, may block tumor growth in different ways by targeting certain cells. It is not yet known whether giving temozolomide together with ipilimumab, nivolumab, or a combination of both is a better treatment for GBM or gliosarcoma.

This study is to determine the optimal dose of single-agent treatment with ipilimumab, nivolumab and the combination when given with temozolomide during maintenance treatment for newly diagnosed GBM.

Additional objectives are (a) to collect and record the side effect profiles for single-agent treatment with nivolumab and the combination of ipilimumab and nivolumab when given with temozolomide during the maintenance phase for newly diagnosed GBM and (b) to perform pilot studies of immune cells within tumor samples, e.g. phenotyping tumor infiltrating lymphocytes (TILs) by interrogating tumor tissues from diagnostic tumor blocks.

Primary outcome measures include (a) immune-related DLTs for the single-agent treatment with nivolumab [Time Frame: Up to 8 weeks]; or (b) immune-related DLTs for the combination of ipilimumab and nivolumab when given with temozolomide [Time Frame: Up to 8 weeks].

Secondary outcome measures include incidence of adverse events, graded using the National Cancer Institute Common Terminology Criteria for Adverse Events version 4.0 [Time Frame: Up to 1 month post-treatment]. The investigators will collect and record the side effect profiles for single-agent treatment with nivolumab and the combination of ipilimumab and nivolumab when given with temozolomide during the maintenance phase for newly diagnosed GBM. Secondary outcome measure will also include biomarker analysis of immune cells within tumor samples using standard immunohistochemistry [Time Frame: Up to 1 month post-treatment]. The results of this battery of assays will be used to obtain pilot data and an assessment of feasibility in obtaining reproducible results in preparation for a subsequent randomized, statistically powered comparative clinical trial.

### Study Arms

### Experimental: Arm I (Temozolomide and Nivolumab)

Within 5 weeks after completion of chemoradiation, patients receive temozolomide PO on days 1-5. Treatment repeats every 28 days for up to 6 courses in the absence of disease progression or unacceptable toxicity. Patients also receive nivolumab IV over 90 minutes once every 2 weeks for 24 weeks and then once every 2 weeks for 42 weeks in the absence unacceptable toxicity.

### Experimental: Arm II (Temozolomide, Nivolumab, Ipilimumab)

Within 5 weeks after completion of chemoradiation, patients receive temozolomide PO on days 1-5. Treatment repeats every 28 days for up to 6 courses in the absence of disease progression or unacceptable toxicity. Patients also receive ipilimumab IV over 90 minutes once every 4 weeks for 6 courses and nivolumab IV over 90 minutes once every 2 weeks for 48 weeks in the absence unacceptable toxicity.

After completion of study treatment, patients are followed up at 1 month, and then every 3 months for 1 year, every 4 months for 1 year, and then every 6 months thereafter.

Subjects eligible for study should be 18 Years and older. Inclusion criteria for the study include:

(a) Histopathologically proven diagnosis of GBM or gliosarcoma within 7 days prior to registration;

(b) The tumor must be unifocal, confined to the supratentorial compartment and have undergone a gross total or near gross total resection;

(c) The formalin-fixed, paraffin-embedded (FFPE) tumor tissue block must be available to be sent for retrospective central pathology review after registration;

(d) Patients must be registered within 28 days of completion of chemoradiation;

(e) History/physical examination within 7 days prior to registration;

(f) Patients must have undergone an evaluation by magnetic resonance imaging (MRI) within 28 days of completing radiation and must also be within 7 days prior to registration; MRI must not demonstrate tumor progression

(g) Karnofsky performance status >=70 within 7 days prior to registration

(h) Absolute neutrophil count >=1,500 cells/mm\hat{ }3

(i) Platelet count >=100,000 cells/mm\hat{ }3

(j) Blood urea nitrogen (BUN)=<30 mg/dl

(k) Serum creatinine=<1.7 mg/dl

(l) Total bilirubin (except patients with Gilbert's syndrome, who are eligible for the study but exempt from the total bilirubin eligibility criterion)=<2.0 mg/dl

(m) Alanine aminotransferase (ALT) and aspartate aminotransferase (AST)=<2.5×upper limit of normal (ULN)

(n) The patient must have completed chemoradiation (all cohorts) within standards of care established by prior Radiation Therapy Oncology Group (RTOG)/NRG Oncology studies as follows:
(i) Radiation therapy
Modality: either 3-dimensional (3D) or intensity-modulated radiation therapy (IMRT), or proton therapy is allowed;
Time to Initiation: radiotherapy must be initiated within or equal to 35 days after surgery
Target Volumes: target volume definition will be based upon postoperative-enhanced magnetic resonance imaging (MRI); preoperative imaging should be used for correlation and improved identification, as necessary
Dose Guidelines: the initial target volume will be treated to 46 Gy in 23 fractions; after 46 Gy, the conedown or boost volume will be treated to a total of 60 Gy, with seven additional fractions of 2 Gy each (14 Gy boost dose)
(ii) Temozolomide during concomitant radiation therapy
Temozolomide must have been administered continuously from day 1 of radiotherapy to the last day of radiation at a daily oral dose of 75 mg/m\hat{ }2 for a maximum of 49 days

(o) The patient must not be on a corticosteroid dose greater than physiologic replacement dosing defined as 30 mg of cortisone per day or its equivalent; and

(p) The patient must provide study-specific informed consent prior to study entry.

Exclusion Criteria include:

(a) Definitive clinical or radiologic evidence of progressive disease;

(b) Prior placement of Gliadel wafer or local brachytherapy;

(c) Use of an immunotherapy such as a vaccine therapy, dendritic cell vaccine or intracavitary or convectional enhanced delivery of therapy;

(d) Prior invasive malignancy (except non-melanomatous skin cancer) unless disease free for a minimum of 3 years;

(e) Unstable angina within the last 6 months prior to registration;

(f) Transmural myocardial infarction within the last 6 months prior to registration;

(g) Evidence of recent myocardial infarction or ischemia by the findings of S-T elevations of >=2 mm using the analysis of an electrocardiogram (EKG) performed within 7 days prior to registration;

(h) New York Heart Association grade II or greater congestive heart failure requiring hospitalization within 12 months prior to registration;

(i) History of stroke, cerebral vascular accident (CVA) or transient ischemic attack within 6 months prior to registration;

(j) Serious and inadequately controlled cardiac arrhythmia;

(k) Significant vascular disease (e.g., aortic aneurysm, history of aortic dissection) or clinically significant peripheral vascular disease;

(l) Evidence of bleeding diathesis or coagulopathy;

(m) Serious or non-healing wound, ulcer, or bone fracture or history of abdominal fistula, gastrointestinal perforation, intra-abdominal abscess major surgical procedure, open biopsy, or significant traumatic injury within 28 days prior to registration, with the exception of the craniotomy for tumor resection;

(n) Acute bacterial or fungal infection requiring intravenous antibiotics at the time of registration;

(o) Chronic obstructive pulmonary disease exacerbation or other respiratory illness requiring hospitalization or precluding study therapy at the time of registration;

(p) Hepatic insufficiency resulting in clinical jaundice and/or coagulation defects; note, however, that laboratory tests for additional liver function tests and coagulation parameters are not required for entry into this protocol;

(q) Acquired immune deficiency syndrome (AIDS) based upon current Centers for Disease Control and Prevention (CDC) definition; note, however, that human immunodeficiency virus (HIV) testing is not required for entry into this protocol;

(r) Active connective tissue disorders, such as lupus or scleroderma, which in the opinion of the treating physician may put the patient at high risk for immunologic toxicity;

(s) Patients with active autoimmune disease or history of autoimmune disease that might recur, which may affect vital organ function or require immune suppressive treatment including systemic corticosteroids, should be excluded; these include but are not limited to patients with a history of immune related neurologic disease, multiple sclerosis, autoimmune (demyelinating) neuropathy, Guillain-Barre syndrome or chronic inflammatory demyelinating polyneuropathy (CIDP), myasthenia gravis; systemic autoimmune disease such as systemic lupus erythematosus (SLE), connective tissue diseases, scleroderma, inflammatory bowel disease (IBD), Crohn's, ulcerative colitis, hepatitis; and patients with a history of toxic epidermal necrolysis (TEN), Stevens-Johnson syndrome, or phospholipid syndrome should be excluded;
(i) Of note, patients with vitiligo, endocrine deficiencies including thyroiditis managed with replacement hormones including physiologic corticosteroids are eligible; patients with rheumatoid arthritis and other arthropathies, Sjögren's syndrome and psoriasis controlled with topical medication and patients with positive serology, such as antinuclear antibodies (ANA), anti-thyroid antibodies should be evaluated for the presence of target organ involvement and potential need for systemic treatment but should otherwise be eligible;

(t) Any other major medical illnesses or psychiatric impairments that in the investigator's opinion will prevent administration or completion of protocol therapy;

(u) Pregnancy or lactating females; women of childbearing potential must have a negative serum pregnancy test within 7 days prior to registration;

(v) Patients who are HIV positive are not eligible; note also that HIV testing is not required for eligibility for this protocol; however a history of HIV evokes this ineligibility; and

(w) History of severe hypersensitivity reaction to any monoclonal antibody.

### Example 10

A Randomized Phase 3 Open Label Study of Nivolumab Vs Temozolomide Each in Combination with Radiation Therapy in Newly Diagnosed Adult Subjects with Unmethylated MGMT (Tumor O-6-Methylguanine DNA Methyltransferase) GBM

The primary objectives of this study are to compare the overall survival (OS) of subjects receiving nivolumab plus radiation therapy (RT+nivolumab) versus temozolomide plus radiation therapy (RT+TMZ) in subjects with newly-diagnosed GBM and unmethylated MGMT tumors after surgical resection. Unmethylated MGMT subjects represent the GBM population with the highest unmet medical need. In a retrospective study of newly diagnosed GBM patients treated with chemoradiation, subjects with unmethylated MGMT GBM tumors rapidly progress and have a median overall survival of only 12-15 months (vs methylated MGMT, with median OS of 22-26 months).

The secondary objectives of this study are 1) to compare the investigator-assessed progression-free survival (PFS) of RT+nivolumab v. RT+TMZ, and 2) to estimate the overall survival at 24 months (OS[24] of RT+nivolumab versus RT+TMZ).

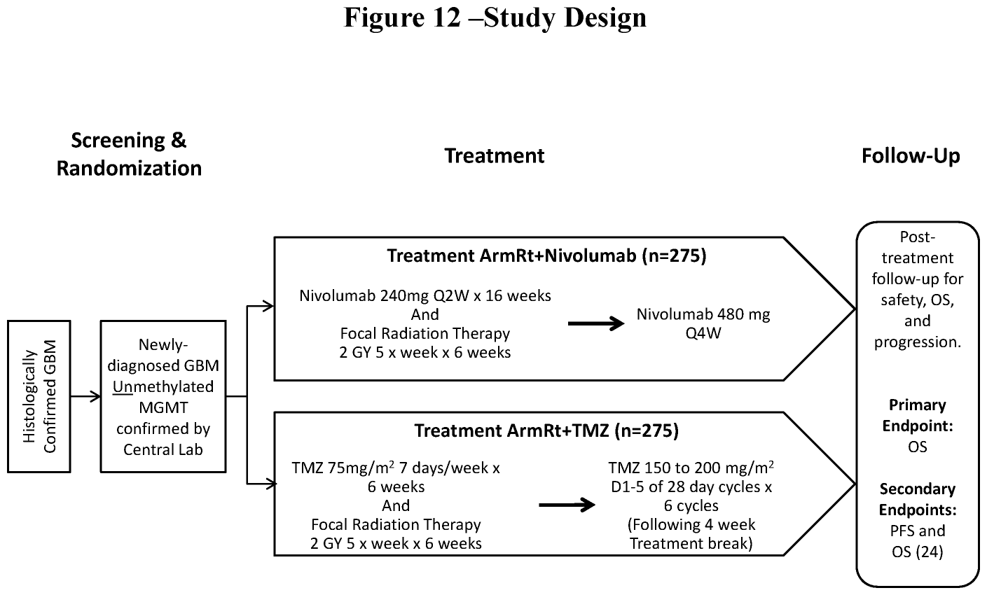

As can be seen in FIG. 12 and Table 7, Nivolumab is administered 240 mg IV as a 30 minute infusion every 2 weeks for 16 weeks followed by nivolumab 480 mg as a 30 minute infusion every 4 weeks beginning at Week 17 until progression, unacceptable toxicity, withdrawal of consent, or the study ends, whichever comes first or Temozolomide is administered 75 mg/m² orally daily during radiation therapy followed by 4 week break then 6 (28-day) cycles temozolomide dosed on Days 1-5 at 150 mg/m² in cycle 1 increasing to 200 mg/m² as tolerated up to 6 cycles.

TABLE 12
Dosing Schedules for RT + Nivolumab and RT + TMZ

| | | Frequency of | Route of | |
|---|---|---|---|---|
| Drug | Dose | Administration | Administration | Duration |
| Arm RT + | 240 mg | Every 2 weeks, | 30 minute | Until progression, |
| Nivolumab | 480 mg, after 16 | weeks 1-15 | intravenous | unacceptable |
| | weeks of dosing | Every 4 weeks after | (IV) | toxicity, or |
| | | week 16 | | discontinuation |
| | | | | from treatment |
| Arm RT + TMZ | 75 mg/m2 daily | Daily from first day | Oral (PO) | Until completion |
| (Temozolomide) | during RT then 150 | of RT to last day of | | of dosing, |
| | mg/m2 D 1-5 | RT (not to exceed | | progression, |
| | for C1 and increased | 49 days) then 4 | | unacceptable |
| | to 200 mg/m2 D 1-5 | week break | | toxicity or |
| | for C2-C6 as | followed by TMZ | | discontinuation |
| | tolerated | daily for 5 days | | from treatment |
| | | every 28 days × 6 | | |
| | | cycles³ | | |

Subjects randomized to nivolumab should begin treatment within 3 days of randomization and continue at a dose of 240 mg as a 30-minute IV infusion on Day 1 of each treatment cycle every 2 weeks, until progression, unacceptable toxicity, withdrawal of consent, or the study ends, whichever comes first. Subjects randomized to the RT+nivolumab arm that remain on nivolumab therapy for 16 weeks will transition to nivolumab 480 mg as a 30-minute IV infusion administered every 4 weeks (q4w) beginning at Week 17. If required for clinic scheduling, subjects may be dosed up to 3 days before or after the scheduled date, i.e., at intervals of not less than 12 days or more than 17 days. A dose given more than 3 days after the intended dose date will be considered a delay. Subsequent dosing should be based on the actual date of administration of the previous dose of drug. ³ Additional cycles of temozolomide are permitted for subjects enrolled in Japan.

Subjects on RT+TMZ will be administered temozolomide (TMZ, TEMODAR®) daily during RT and as maintenance therapy for 6 cycles. Temozolomide (TMZ) will be dosed at 75 mg/m₂ once per day continuously throughout RT, typically for 42 days and with a maximum of 49 days during concomitant RT+TMZ dosing. After completion of RT, there will be a 4 week break. Subjects will receive 6 cycles of temozolomide daily×5 days every 28 days. Dose levels for TMZ maintenance monotherapy are in Table 13.

TABLE 13
Dose levels for TMZ maintenance monotherapy.

| Dose level | TMZ Dose in mg/m²/day | Notes |
|---|---|---|
| −1 | 100 mg/m²/day | Reduction for prior toxicity |
| 0 | 150 mg/m²/day | Dose during Cycle 1 |
| 1 | 200 mg/m²/day | Dose during C2-6 in absence |
| | | of TMZ-related |
| | | toxicity |

In the first maintenance cycle TMZ is given at a dose of 150 mg/m², and then increased to 200 mg/m² in cycle 2 as follows: If, during the first cycle, all non-hematologic AEs observed were grade ≤2 (except alopecia, nausea and vomiting) and with platelets ≥100×109/L and ANC≥1.5×10⁹/L, then the temozolomide dose should be escalated to dose level 1 and this dose should be used as the starting dose for subsequent cycles. If treatment after cycle 1 has to be delayed because of ongoing non-hematologic AEs of grade ≥2, then no escalation is possible. If the dose was not escalated at cycle 2, then the dose should not be escalated in further cycles based on adverse events. If there was a prior dose reduction during the concomitant period with RT, then dose −1 should be the starting dose for subsequent cycles.

Maintenance treatment begins following a 4 week treatment break after RT. TMZ treatment may begin if blood count (obtained within the prior 3 days) shows ANC≥1.5×10⁹/L, platelet count ≥100×109/L and any Grade ≥3 non-hematologic AE (except alopecia) must have resolved to grade ≤1). If AEs persist, treatment should be delayed by 1 week for up to 4 consecutive weeks. If, after 4 weeks of delay, all AEs have still not resolved to ≤grade 1: then any further adjuvant treatment with temozolomide should be discontinued.

The exploratory objectives of the study are 1) to evaluate the safety of RT+nivolumab and RT+TMZ treatment arms; 2) to evaluate health-related quality of life using the EQ-5D and the European Organization for Research and Treatment of Care General Cancer Module (QLQ-C30) and brain cancer module (QLQ-BN20); 3) to assess neurologic functioning in the RT+nivolumab and RT+TMZ treatment arms using the Neurologic Assessment in Neuro-Oncology (NANO) Scale; 4) to assess cognition in the RT+nivolumab and RT+TMZ treatment arms using the Cogstate tool; 5) to evaluate the pharmacodynamic activity of nivolumab in the peripheral blood and tumor tissue as measured by flow cytometry, immunohistochemistry, soluble factor analysis, and gene expression (microarray technology, quantitative RT-PCR); 7) to investigate the association between biomarkers in the peripheral blood and tumor tissue such as PD-L1 expression, with safety and efficacy for subjects with newly diagnosed GBM treated with nivolumab; 8) to characterize the PK of nivolumab and explore exposure-response relationships; and 9) to characterize the immunogenicity of nivolumab in this setting.

This study will enroll subjects with newly-diagnosed GBM, following surgical resection of the tumor. After informed consent is obtained, subjects will enter the screening phase. Tumor tissue will be evaluated for MGMT methylation by a central laboratory assay. In order to randomize 550 eligible subjects, a total of approximately 1200 subjects are expected to have tumor screening, of which approximately 600 subjects are anticipated to have unmethylated MGMT tumors.

Subjects with a central laboratory result of unmethylated MGMT may continue in the screening phase, in which eligibility for randomization will be documented and baseline demographic and disease information submitted; for details. During the screening phase a contrast-enhanced MRI should be obtained within 48 hours post-surgery (within 24 hours preferred).

When ready to begin study treatment, subjects will proceed to the treatment phase of the study. All subjects who enter the treatment phase, i.e., all randomized subjects, will be followed for safety and tolerability, tumor progression and survival. A contrast-enhanced MRI should be performed 4 weeks after completing radiation therapy, then every 8 weeks (±1 week) until progression regardless of treatment schedule. Tumor progression will be assessed using Radiologic Assessment in Neuro-Oncology criteria (RANO). Additional assessments will be performed for cognitive function, neurologic function, biomarkers, and patient-reported quality of life outcomes.

After cessation of study treatment for any reason, all randomized subjects will enter a follow up phase. In the short term, visits are defined for reporting of treatment-related adverse events. All subjects in whom disease progression had not been detected at the time study treatment is stopped will be followed closely for progression with contrast enhanced MRI every 8 weeks (±1 week). After progression subsequent treatment will be reported. All randomized subjects MUST be followed for survival (primary endpoint).

The total time elapsed from diagnostic surgery to initiation of RT may not exceed 42 days including a maximum of 7 days from randomization to initiation of RT. Subjects in the RT+nivolumab arm should begin nivolumab within 3 days after randomization, and may be given at any time prior to RT start, as clinically appropriate. Subjects randomized to the RT+TMZ arm will begin combination chemo-radiotherapy on the same day, up to 7 days after randomization, as clinically appropriate.

Radiation therapy should begin after substantial recovery from surgical resection, not more than 42 days after surgery. External-beam RT to a total dose of 60 Gy will be administered in daily doses of 2 Gy, typically on a 5 days on/2 days off schedule as appropriate for scheduling, over 6-7 weeks. RT is administered to the pre-operative tumor volume plus a 2-3 cm margin, as directed by a radiation oncologist. Suspected progression occurring within 12 weeks after RT may be “pseudoprogression”; in this setting, suspected progression should be confirmed prior to discontinuation of treatment.

The follow-up phase begins when the decision is made to discontinue a subject from study treatment, including for adverse events, for maximum clinical benefit (investigator decision), for subject request, for completion of TMZ maintenance therapy (6 cycles), disease progression, or another reason.

Subjects will be evaluated for adverse events with visits at 35 days (±7 days) and 115 days (±7 days) after last dose documented for a minimum of 100 days after discontinuation, but drug-related toxicities should continue be followed until they resolve, return to baseline or are deemed irreversible.

Subjects who discontinue treatment for a reason other than disease progression will continue to undergo tumor assessments and NANO scale until progression (secondary study endpoint). Introduction of anti-tumor therapy in the absence of progression is strongly discouraged.

All subjects MUST be followed for survival (primary study endpoint). Subsequent treatment, if any, will be reported.

The Inclusion Criteria are as follows:

Signed informed written consent:
Written informed consent and HIPAA authorization (applies to covered entities in the US only) obtained from the subject/legal representative prior to performing any protocol-related procedures;
Subjects must be willing and able to comply with scheduled visits, treatment schedule, laboratory testing, and other requirements of the study.

Target Population:
Newly diagnosed histologically confirmed supratentorial GBM (Grade 4 malignant glioma by World Health Organization including gliosarcoma)
a) No treatment for GBM other than surgery;
b) Postoperative baseline MRI within 48 hours (preferably 24 hours) of surgical resection
Full recovery from surgical resection;
a) No major ongoing safety issues following surgery
b) ≤20 mg prednisone daily or ≤3 mg dexamethasone daily (or equivalent)
Centrally confirmed unmethylated MGMT GBM;
Karnofsky performance status of ≥70;
Eligible for radiation therapy based on NCCN guidelines.
Subject may reenroll in the study.

Age and reproductive status:
Males and Females age ≥18 years old;
Women of childbearing potential (WOCBP) must have a negative serum or urine pregnancy test (minimum sensitivity 25 IU/L or equivalent units of HCG) within 24 hours prior to the start of study drug;
Women must not be breastfeeding;
Women of childbearing potential (WOCBP) must agree to follow instructions for method(s) of contraception for a period of 30 days (duration of ovulatory cycle) plus the time required for the investigational drug to undergo five half lives. The terminal half life of nivolumab is up to 25 days. The terminal half-life of temozolomide is 1.2 hours
   WOCBP randomized to receive nivolumab should use an adequate method to avoid pregnancy for 23 weeks (30 days plus the time required for nivolumab to undergo five half-lives) after the last dose of investigational drug. WOCBP randomized to receive temozolomide should use an adequate method to avoid pregnancy for 6 weeks (30 days plus the time required for temozolomide to undergo five half lives) after last dose of temozolomide.
Males who are sexually active with WOCBP must agree to follow instructions for method(s) of contraception for a period of 90 days (duration of sperm turnover) plus the time required for the investigational drug to undergo five half lives. The terminal half-life of nivolumab is up to 25 days.
   Males randomized to receive nivolumab who are sexually active with WOCBP must continue contraception for 31 weeks (90 days plus the time required for nivolumab to undergo five half-lives) after the last dose of investigational drug. Males randomized to temozolomide should be advised not to father a child up to 6 months after receiving the last dose and to seek advice on cryoconservation of sperm prior to treatment, because of the possibility of irreversible infertility due to therapy with TMZ.
Azoospermic males and WOCBP who are continuously not heterosexually active are exempt from contraceptive requirements. However WOCBP must still undergo pregnancy testing as described in this section.
Investigators shall counsel WOCBP and male subjects who are sexually active with WOCBP on the importance of pregnancy prevention and the implications of an unexpected pregnancy. Investigators shall advise WOCBP and male subjects who are sexually active with WOCBP on the use of highly effective methods of contraception. Highly effective methods of contraception have a failure rate of <1% when used consistently and correctly.
At a minimum, subjects must agree to the use of two methods of contraception, with one method being highly effective and the other method being either highly effective or less effective as found in the informed consent.

Physical and Laboratory Test Findings:
WBC≥2,000/μL;
Neutrophils ≥1,500/μL;
Platelets ≥100×10³/μL;
Hemoglobin ≥9.0 g/dL;
Serum creatinine ≤1.5×ULN or creatinine clearance (CrCl)≥50 mL/min (using the Cockcroft-Gault formula)
   Female CrCl=(140-age in years)×weight in kg×0.85 72×serum creatinine in mg/dL
   Male CrCl=(140-age in years)×weight in kg×1.00 72×serum creatinine in mg/dL
AST≤3.0×ULN;
ALT≤3.0×ULN;
Total Bilirubin ≤1.5×ULN (except subjects with Gilbert Syndrome who may have a total bilirubin <3.0×ULN).

The Exclusion Criteria are as follows:

Target Disease Exceptions:
Prior treatment for GBM (other than surgical resection);
Recurrent GBM;
MGMT methylated, partially methylated, or indeterminate GMB;
Biopsy only of GBM at surgery, defined as <20% resection;
Ongoing requirement for supraphysiologic steroid defined as >20 mg prednisone daily or >3 mg dexamethasone daily (or equivalent), due to intracranial mass effect;
CNS hemorrhage of Grade >1 on baseline MRI scan, unless subsequently documented to have resolved;
Any known metastatic extracranial or leptomeningeal disease;
Secondary GBM (i.e., progression from prior low-grade or anaplastic astrocytoma); and
Known IDH mutated tumor (if available; test not required).

Medical History and Concurrent Diseases:
Any serious or uncontrolled medical disorder that, in the opinion of the investigator, may increase the risk associated with study participation or study drug administration, impair the ability of the subject to receive protocol therapy, or interfere with the interpretation of study results;
Prior treatment with an anti-PD-1, anti-PD-L1, anti-PD-L2, anti-CTLA-4 antibody or any other antibody or drug specifically targeting T-cell co-stimulation or immune checkpoint pathways;
Subjects with an active, known or suspected autoimmune disease. Subjects with type I diabetes mellitus, hypothyroidism only requiring hormone replacement, skin disorders (such as vitiligo, psoriasis or alopecia) not requiring systemic treatment, or conditions not expected to recur in the absence of an external trigger are permitted to enroll;
Subjects with a condition requiring systemic treatment with either corticosteroids (>20 mg daily prednisone or >3 mg dexamethasone or equivalent) or other immunosuppressive medications within 14 days of randomization. Inhaled or topical steroids, and adrenal replacement steroid doses >20 mg daily prednisone or >3 mg dexamethasone or equivalent, are permitted in the absence of active autoimmune disease;
Subjects with interstitial lung disease that is symptomatic or may interfere with the detection or management of suspected drug-related pulmonary toxicity;
Subjects with history of life-threatening toxicity, including hypersensitivity reaction, related to prior immunoglobulin treatment for another condition (except those considered unlikely to re-occur, with written approval of medical monitor) or any other study drug component;
History or evidence upon physical/neurological examination of other central nervous system condition (e.g., seizures, abscess) unrelated to cancer, unless adequately controlled by medication or considered not potentially interfering with protocol treatment;
Surgical procedure <7 days prior to study treatment, vascular access device no restriction;
Subjects unable (e.g., due to pacemaker or ICD device) or unwilling to have a contrast-enhanced MRI of the head;
History of allergy or hypersensitivity to study drug components;
Unable to swallow oral medication or any gastrointestinal disease or surgical procedure that may impact the absorption of study drug.

Physical and Laboratory Test Findings:
Any positive test for hepatitis B virus or hepatitis C virus;
Known history of positive test for human immunodeficiency virus (HIV) or known acquired immunodeficiency syndrome (AIDS). NOTE: Testing for HIV must be performed at sites where mandated by local regulation.

Allergies and Adverse Drug Reaction:
History of allergy or hypersensitivity to study drug components

Other Exclusion Criteria:
Prisoners or subjects who are involuntarily incarcerated (Note: under certain specific circumstances a person who has been imprisoned may be included or permitted to continue as a subject. Strict conditions apply and approval is required.);
Subjects who are compulsorily detained for treatment of either a psychiatric or physical (e.g., infectious disease) illness.

Concomitant treatments. It is expected that enrolled subjects will have systemic corticosteroids tapered as quickly as clinically appropriate during screening phase, and discontinued if possible prior to randomization. Supportive care for all disease-related or treatment-related adverse events should be maximized for all subjects on this study.

Prohibited and/or restricted treatments. Concurrent anti-neoplastic therapy, including other chemotherapy, immunotherapy, additional radiation therapy or investigational agents for treatment of GBM are prohibited during the treatment phase. Use of any additional noninvasive medical device treatment of GBM (e.g., novoTTF, OPTUNE©) is prohibited.

Immunosuppressive agents, including systemic corticosteroids, are prohibited during study treatment unless utilized to treat a drug-related adverse event. Subjects with a condition requiring systemic treatment with either corticosteroids (>20 mg daily prednisone or >3 mg dexamethasone per day (or equivalent) or other immunosuppressive medications (including within 14 days of randomization) are excluded. Subjects continuing to require supra-physiologic steroids (prednisone >20 mg daily or >3 mg dexamethasone per day or equivalent) may not be randomized. Inhaled or topical steroids, and adrenal replacement steroid doses ≤20 mg daily prednisone or ≤3 mg dexamethasone per day or equivalent, are permitted in the absence of active autoimmune disease.

Use of valproic acid is prohibited on the RT+TMZ arm; the subject must be transitioned to an alternative anticonvulsant.

Discontinuation of subjects following any treatment with study drug. Subjects MUST discontinue investigational product (and non-investigational product at the discretion of the investigator) for any of the following reasons
Disease progression, except as otherwise described;
Maximum clinical benefit, as determined by investigator;
Any clinical adverse event (AE), laboratory abnormality or intercurrent illness which, in the opinion of the investigator, indicates that continued participation in the study is not in the best interest of the subject;
Subject's request to stop study treatment;
Completion of 6 cycles of maintenance TMZ;
Termination of the study by Bristol-Myers Squibb (BMS);
Loss of ability to freely provide consent through imprisonment or involuntarily incarceration for treatment of either a psychiatric or physical (e.g., infectious disease) illness;
Initiation of antineoplastic therapy other than as randomized.

As this is a survival study, subjects discontinuing study treatment will remain on study for documentation of progression and death.

### Study Assessments:

Safety Evaluation: Adverse events will be assessed continuously during the study and for 100 days post last treatment. Adverse events will be coded using the most current version of MedDRA and reviewed for potential significance and importance. Adverse events will be evaluated according to the NCI CTCAE Version 4.03. Subjects should be followed until all treatment-related adverse events have recovered to baseline or are deemed irreversible by the investigator.

Efficacy Assessments: Baseline Contrast-enhanced MRI within 48 hours post-surgery (within 24 hours preferred) will be performed on an MRI qualified to meet study specific acquisition parameters. Tumor imaging assessments will occur 4 weeks after completion of radiotherapy then every 8 weeks (+/−1 wk) thereafter until disease progression. In addition, NANO scale will be completed prior to dosing Day 1 Week 1 and then with each MRI. Subjects will be treated until unacceptable toxicity or disease progression. Following discontinuation of therapy, safety will be assessed through post-treatment Follow Up visit 2 (˜100 days from last dose). Survival status will be assessed every 3 months after follow up visits are completed, and may be completed via telephone or in person visits.

### Statistical Considerations:

Sample Size: Approximately 550 subjects will be randomized to the two arms (RT+nivolumab vs RT+TMZ) in a 1:1 ratio, stratified by complete or partial resection at baseline.

This study requires at least 397 events (i.e., death), with an interim analysis after at least 298 events (75% of total OS events), to provide 90% power to detect a hazard ratio (HR) of 0.72 with an overall type 1 error of 0.05 (two-sided). The stopping boundaries at the interim and final analyses will be based on the actual number of OS events at the time of the analysis using Lan DeMets alpha spending function with O'Brien-Fleming boundaries. If the interim and final analyses occur exactly at the planned number of events, the projected alpha level will be 0.019 and 0.044, for the interim and the final analyses, respectively. This translates to an observed HR of 0.762 (median OS of 13.0 vs 17.0 months) and 0.817 (median OS of 13.0 vs 15.9 months) or less resulting in a statistically significant improvement at the interim and final analyses respectively.

The secondary endpoint of only PFS will be tested hierarchically.

Endpoints: The primary endpoint is overall survival (OS). OS is defined as the time between the date of randomization and the date of death due to any cause. A subject who has not died will be censored at the last known alive date. OS will be followed continuously while subjects are on study drug and every 3 months via in-person or phone contact during survival follow-up phase of the study.

The secondary endpoint of PFS is defined as the time from randomization to the date of the first documented tumor progression or death due to any cause. Subjects who die without a reported progression will be considered to have progressed on the date of death. Subjects who did not have disease progression or die will be censored at the date of last tumor assessment. Subjects who did not have any on study tumor assessment and did not die will be censored at the randomization date. Subjects who started any subsequent anti-cancer therapy without a prior reported progression will be censored at the last tumor assessment prior to initiation of the subsequent anti-cancer therapy. Subjects who had surgical resection post start of study treatment will be censored at the last tumor assessment date prior to initiation of surgical resection. PFS will be determined by investigator reported response based on RANO criteria.

The secondary endpoint of survival rate at 24 months, is defined as Kaplan-Meier survival probability at 24 months.

### Analyses:

The analyses of primary endpoint of OS and secondary endpoints of PFS and OS[24] will be based on all randomized subjects. A group sequential testing procedure will be applied to OS to control the overall type I error from interim and final analyses. If superiority in OS is demonstrated, a hierarchical hypothesis testing approach for the secondary endpoint of PFS will be used to preserve a study-wise type I error rate at 0.05. OS[24] will be estimated using Kaplan Meier method.

OS and PFS distribution in all randomized subjects will be compared between treatment groups using a two-sided stratified log-rank test. The hazard ratio (HR) and corresponding two-sided 100×(1−adjusted α)% confidence intervals (CIs) will be estimated in a Cox proportional hazard model using treatment as a single covariate. Stratified by complete or partial resection at baseline.

OS and PFS curves will be estimated using the Kaplan-Meier product-limit method. Median OS and PFS along with the corresponding two-sided 95% CIs using the log-log transformation will be computed.

OS [24] and the corresponding 95% CIs using the log-log transformation will be computed after all subjects have follow-up of at least 24 months.

### Endpoints.

Primary Endpoint(s). The primary endpoint is overall survival (OS). OS is defined as the time between the date of randomization and the date of death due to any cause. A subject who has not died will be censored at the last known alive date. OS will be followed continuously while subjects are on study drug and every 3 months via in-person or phone contact during survival follow-up phase of the study.

Second Endpoint(s). The first secondary objective (comparing PFS between arm RT+Nivolumab and RT+TMZ) will be measured by the endpoint of PFS. PFS is defined as the time from randomization to the date of the first documented tumor progression or death due to any cause. Subjects who die without a reported progression will be considered to have progressed on the date of death. Subjects who did not have disease progression or die will be censored at the date of last tumor assessment. Subjects who did not have any on study tumor assessment and did not die will be censored at the randomization date. Subjects who started any subsequent anti-cancer therapy without a prior reported progression will be censored at the last tumor assessment prior to initiation of the subsequent anti-cancer therapy. Subjects who had surgical resection post start of study treatment will be censored at the last tumor assessment date prior to initiation of surgical resection PFS will be determined by investigator reported response based on RANO criteria.

Exploratory Endpoints.
Safety and tolerability will be measured by the incidence of adverse events, serious adverse events, deaths and laboratory abnormalities.
The evaluation of NANO will be measured by the distribution of change from baseline in the level of function score for each domain.
Pharmacokinetics parameters will be estimated using serum concentration-time data. The evaluation of HRQoL will be measured by changes from baseline in the EORTC-QLQ-C30 global health status/QoL composite scale and by changes from baseline in the remaining EORTC QLQ-C30 and BN-20 scales. All scales and single items are scored on a categorical scales and linearly transformed to 0-100 scales with higher scores for a functional scale representing higher levels of functioning, higher scores for the global health status/quality of life representing higher levels of global health status/quality of life and higher scores for a symptom scale representing higher level of symptoms. The evaluation of HRQoL on general health status will be measured by changes from baseline in the EQ-5D scales.
Cognition will be assessed using changes from baseline in neurocognitive functioning, cognitive deficits and/or executive functioning using Cogstate. Neurologic functioning will be assessed using NANO scale.

The present application claims benefit to U.S. Provisional Application Nos. 62/092,783, filed Dec. 16, 2014 and 62/261,130, filed Nov. 30, 2015; which are incorporated herein by reference in their entireties.

1. A method for treating a subject afflicted with a glioma comprising administering to the subject a therapeutically effective amount of an antibody or an antigen-binding portion thereof that binds specifically to a Programmed Death-1 (PD-1) receptor and inhibits PD-1 activity (“anti-PD-1 antibody”).

2. The method of claim 1, wherein the glioma is selected from the group consisting of: astrocytoma, brainstem glioma, ependymoma, mixed glioma, oligodendroglioma, and optic nerve glioma.

3. The method of claim 2, wherein the glioma is glioblastoma.

4-7. (canceled)

8. The method of claim 1, wherein the anti-PD-1 antibody is nivolumab.

9-10. (canceled)

11. The method of claim 10, wherein the subject is further treated with an anti-cancer therapy concurrently with, prior to, or after the administering, wherein the anti-cancer therapy comprises a therapy selected from administering an anti-cancer agent, a radiation therapy, a surgery, and any combination thereof.

12-14. (canceled)

15. The method of claim 11, wherein the anti-cancer agent comprises an alkylating agent, a second antibody or an antigen-binding portion thereof, or both.

16. The method of claim 15, wherein the alkylating agent comprises temozolomide.

17. (canceled)

18. The method of claim 11, wherein the anti-cancer agent comprises a second antibody, wherein the second antibody binds specifically to Cytotoxic T-Lymphocyte Antigen-4 (CTLA-4) and inhibits CTLA-4 activity (“anti-CTLA-4 antibody”).

19-21. (canceled)

22. The method of claim 18, wherein the anti-CTLA-4 antibody is ipilimumab.

23. (canceled)

24. The method of claim 1, wherein the anti-PD-1 antibody is administered at a dose of about 0.1 mg/kg to about 10 mg/kg body weight.

25. (canceled)

26. The method of claim 1, wherein the anti-PD-1 antibody is administered at a dose of about 3 mg/kg body weight or about 1 mg/kg body weight.

27. The method of claim 1, wherein the anti-PD-1 antibody is administered at a flat dose of about 240 mg, about 360 mg, or about 480 mg.

28. The method of claim 1, wherein the anti-PD-1 antibody is administered once about every 2 weeks.

29. The method claim 1, wherein the anti-PD-1 antibody is administered at a flat dose of about 240 mg once about every 2 weeks.

30. The method of claim 1, wherein the anti-PD-1 antibody is administered at a flat dose of about 240 mg once about every 2 weeks for about 16 weeks, and wherein the anti-PD-1 antibody is administered at a flat dose of about 480 mg every 4 weeks thereafter.

31. The method of claim 18, wherein the anti-CTLA-4 antibody is administered at a dose of about 0.1 mg/kg to about 10 mg/kg body weight once about every 1, 2, 3, or 4 weeks.

32-37. (canceled)

38. The method of claim 18, wherein the anti-PD-1 antibody is administered at a dose of about 3 mg/kg body weight and the anti-CTLA-1 antibody is administered at a dose of about 1 mg/kg body weight.

39-42. (canceled)

43. The method of claim 18, wherein the anti-PD-1 and the anti-CTLA-4 antibodies are formulated for intravenous administration.

44-51. (canceled)

52. A kit for treating a subject afflicted with a glioma, the kit comprising:
(a) a dosage ranging from about 0.1 mg/kg to about 10 mg/kg body weight of an anti-PD-1 antibody;
(b) a dosage of an anti-cancer agent which is
(i) temozolomide; or
(ii) a dosage ranging from 0.1 to 10 mg/kg body weight of an anti-CTLA-4 antibody; and
(c) instructions for using the anti-PD-1 antibody and the anti-cancer agent in the method of any of claims 16 or 18 to 51.

53. The method of claim 3, wherein the glioblastoma is unmethylated tumor O-6-methylguanine DNA methyltransferase (MGMT) glioblastoma.

54. (canceled)
